(19) United States
(12) Patent Application Publication
(10) Pub. No.: US 2025/0018047 A1
(43) Pub. Date: Jan. 16, 2025

(54) METHODS AND COMPOSITIONS FOR TARGETED PROTEIN DEGRADATION

(71) Applicant: Ranok Therapeutics (Hangzhou) Co. Ltd., Hongzhou (CN)

(72) Inventors: Weiwen Ying, Lexington, MA (US); Chenghao Ying, Shanghai (CN); Kevin P. Foley, Waltham, MA (US); Lingjie Zhang, Wuxi (CN); Mingkai Wang, Hangzhou (CN); Long Ye, Shanghai (CN); Thomas Prince, Danville, PA (US); Wei Yin, Lianyungang (CN); Yaya Wang, Shanghai (CN)

(21) Appl. No.: 18/705,615

(22) PCT Filed: Nov. 7, 2022
(86) PCT No.: PCT/US2022/049155

§ 371 Date: Apr. 29, 2024

(30) Foreign Application Priority Data
Nov. 5, 2021 (WO) PCT/CN2021/129048

(51) Int. Cl. A61K 47/55; A61K 47/54; A61P 35/00

(52) U.S. Cl. CPC A61K 47/55; A61K 47/545; A61P 35/00

(57) ABSTRACT

Provided are PROTACs comprised of a targeting moiety designed to bind KRAS (G12D) linked to a degrader-directing moiety designed to bind an E3 ubiquitin-ligase. Pharmaceutical compositions comprising the disclosed PROTACs and their use in treating cancer and related conditions are also provided.

### RELATED APPLICATIONS

This application claims priority to PCT/CN2021/129048, filed Nov. 5, 2021, the entire contents of which are incorporated herein by reference.

### BACKGROUND

Chemically induced, targeted protein degradation (TPD) is a new modality for small molecule drug development. Small molecules can be designed to induce and/or stabilize the direct physical interactions of target proteins with components of various cellular protein degradation pathways, thereby driving the degradation of the targeted proteins as a way to treat disease. Proteolysis-targeting chimeras (PROTACs) are an example of such small molecules that enable TPD of specific proteins, such as KRAS mutant oncogenes (Burslem and Crews, Cell, 2020, 181:102-114; Pettersson and Crews, Drug Discov Today Technol, 2019, 31:15-27).

In a cell, networks of protein components compete and cooperate to maintain and alter the proteome in order to carry-out specific biological processes. Proteostasis is this dynamic regulation protein synthesis, folding, trafficking and degradation inside and outside of cells. Protein degradation is especially critical for proper cell function including proliferation, differentiation, and death (Lang et al, Arch Toxicol, 2021, 95:1943-1970). In cancer protein degradation is often dysregulated with tumor suppressors degraded while oncogenes are stabilized (Hanahan and Weinberg, Cell, 2011, 144: 646-74).

The ubiquitin-proteasome system (UPS) is the primary pathway cells utilize to degrade and metabolically recycle proteins (Yu and Matouschek, Annu Rev Biophys, 2017, 46:149-173; Navon and Ciechanover, J Biol Chem, 2009, 284:33713-33718). Covalent attachment of the globally expressed 76 amino acid-residue peptide, ubiquitin, marks a substrate protein for degradation. The process of ubiquitination involves a series of enzymatic hand-offs. Initially, ubiquitin is attached to an E1 ubiqutin-activating enzyme. Once activated, ubiquitin is next transferred from E1 to an E2 ubibuitin-conjugating enzyme. Finally, one of several hundred E3 ubiquitin-ligase enzyme complex components, such as cereblon (CRBN) and von Hippel Lindau (VHL) (Bricelj et al, Front Chem, 2021, 9:707317), facilitate the transfer of ubiquitin to a lysine on the substrate protein. Additional ubiquitin peptides may then be attached to lysines on each ubiquitin peptide creating a polyubiquitin chain that directs the substrate protein to the proteosome, a multi-subunit protein degrading enzyme complex. For some protein substrates, ubiquitination may also affect a protein's activity, subcellular localization and protein-protein interaction profile (Zou et al., Int J Mol Sci, 2021, 22:5754; Amm et al., Biochim Biophys Acta, 2014, 1843:182-196;).

The CRBN and VHL proteins function as critical components in Cullin RING E3 ubiquitin-ligase complexes (Cai and Wang, Cell Div, 2016, 11; Cheng et al., Biochim Biophys Acta Rev Cancer, 2019, 1871:138-159). Both CRBN and VHL are widely expressed across tissues types and evolutionarily conserved among vertebrates. Normally, CRBN has been observed to coordinate the ubiquitination and degradation of ion channels, the MEIS2 developmental transcription factor, the AMPK metabolic-regulating kinase, and glutamine synthase (Jo et al., J Neurochem, 2005, 94:1212-24, Hohberger and Enz, FEBS Lett, 2009, 583:633-7; Fischer et al., Nature, 2014, 12:49-53, Lee et al., J Biol Chem, 2014, 289:23343-52; Nguyen, et al., Mol Cell, 61:809-20). CRBN can also be induced to degrade transcription factors IKZF1 and IKZF3 along with Casein kinase 1A1 by immunomodulatory compounds (Kronke et al. Science, 2014, 343:301-5; Petzold et al., Nature, 2016, 532:127-30). VHL normally ubiquitinates hypoxia-inducible factor 1a (HIF1A), the primary transcription factor responsible for promoting angiogenesis (Kaelin, Nat Rev Cancer, 2008, 8:865-73).

PROTACs are bifunctional molecules that contain two different drug moieties held together by a linker. By design these PROTAC molecules can simultaneously bind both a target protein and an E3 ubiquitin-ligase. Within cells this creates ternary complexes composed of target proteins and E3 ubiquitin-ligases that are held together by PROTAC molecules. This induced proximity of the target proteins and E3 ubiquitin-ligases results in the ubiquitination of the target proteins and subsequent degradation by the proteasome. If the target protein is an oncogene this degradation may result in the death of the cancer cell or the inability to proliferate (Pettersson and Crews, Drug Discov Today Technol, 2019, 31:15-27; Bondeson et al., Cell Chem Biol, 2018, 25:78-87; Gadd et al., Nat Chem Biol, 2017, 13:514-521; Zengerle et al., ACS Chem Biol, 2015, 10:1770-1777).

Compared to conventional biochemical enzyme inhibitors, PROTACs possess several advantages. For example, PROTACs are able to work sub-stoichiometrically by inducing multiple rounds of degradation of target proteins. This is presumably due to PROTAC molecules being released from the proteosome degraded protein to bind another target protein and E3 ubiquitin ligase. This leads to a greater potency compared to each isolated moiety binding to its respective target. Moreover, synthesis and recovery of the target protein function is slower for PROTACs than observed for biochemical inhibitors. PROTACs may also possess improved target selectivity over single moiety biochemical inhibitors. Finally, PROTACs can deplete target proteins that are not responsive to biochemical inhibition by binding accessible pockets that do not affect the biochemical activity of the target but still permit their degradation (Pettersson and Crews, Drug Discov Today Technol, 2019, 31:15-27; Ding et al., Trends Pharmacol Sci, 2020, 41:464-474)..

There is a need to develop improved PROTAC agents that direct the degradation of specific proteins in cancer and other diseases. The Kirsten rat sarcoma virus homolog (KRAS) is small GTPase and among the most frequently mutated genes in human cancers (Pylayeva-Gupta et al., Nat Rev Cancer, 2011, 11:761-774). Mutations that lock KRAS in an active GTP-bound state reprograms cells for perpetual proliferation by continuously stimulating the RAF-MAPK and PI3K-AKT-MTOR pro-growth signaling pathways (Kerk et al., Nat Rev Cancer, 2021, 21:510-525; Nussinov et al., Cancer Res, 2018, 78:593-602). KRAS mutated from glycine (G) at the 12^{th} codon to aspartate (D) creates the chronically active KRAS(G12D) oncogene observed in 6.8% of cancers cases analyzed by next-generation sequencing (Zhou et al., Pathol Oncol Res, 2020, 26:2835-2837). In tumor type specific studies, KRAS(G12D) is associated with poor clinical outcomes and observed in 17% of lung, 14.3% of colorectal, and 48% of pancreatic tumors (Aredo et al., Lung Cancer, 2019, 133:144-150; Olmedillas-López et al., World J Gastroenterol, 2017, 23(39):7087-709; Miglio et al., Pathol Res Pract, 2014, 210:307-11; Gou et al., Br J Cancer, 2020, 22:857-867). Historically, oncogenic KRAS mutants have been considered undruggable (McCormick F, Biochem J, 2019, 476:356-74), however the G12D mutant provides a unique moiety binding space due to the encoding of an acidic amino acid residue (D) in place of an amino acid residue possessing only a hydrogen side-chain (G). This allows for cancer cells that are driven by the KRAS(G12D) mutant to be selectively targeted and degraded by PROTAC agents. It is therefore desirable to develop PROTAC agents directed at degrading KRAS(G12D) in cancer.

### SUMMARY

Provided herein are PROTACs comprised of a targeting moiety designed to bind KRAS (G12D) linked to a degrader-directing moiety designed to bind an E3 ubiquitin-ligase, e.g., CRBN or VHL. Such compounds include those having the Formula I:

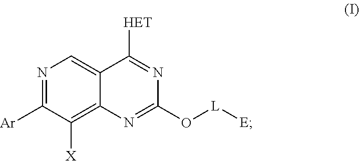

and pharmaceutically acceptable salts thereof. Compositions comprising the disclosed compounds of Formula I as well as methods for their manufacture and their biochemical activity are also provided. In one aspect, the disclosed compounds induce targeted oncogenic protein degradation in a tumor-selective fashion and are useful in the treatment of cancer and related conditions.

### DETAILED DESCRIPTION

### 1. General Description of Compounds

Provided herein are PROTAC compounds having the Formula I:

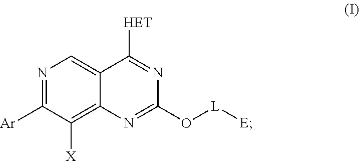

or a pharmaceutically acceptable salt thereof, wherein,
HET is an optionally substituted heterocyclyl;
Ar is an optionally substituted aryl or optionally substituted heteroaryl;
X is hydrogen or halo;
L is a linker; and
E is a chemical moiety that targets E3 ligase.

### 2. Definitions

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.” As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” are used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used herein, the term “alkyl” means a saturated straight chain or branched non-cyclic hydrocarbon having, unless specified otherwise, from 1 to 10 carbon atom e.g., (C₁-C₆)alkyl or (C₁-C₄)alkyl. Representative straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein, the term “alkynyl” means a saturated straight chain or branched non-cyclic hydrocarbon having, unless specified otherwise, from 2 to 10 carbon atoms (e.g., (C₂-C₆)alkynyl or (C₂-C₄)alkynyl) and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like.

As used herein, the term “cycloalkyl” means a saturated, monocyclic alkyl radical having from e.g., 3 to 10 carbon atoms (e.g., from 4 to 6 carbon atoms). Representative cycloalkyls include cyclopropyl, cyclobutyl, cyclopentyl, cyclohexyl, cycloheptyl, cyclooctyl, cyclononyl, and cyclodecanyl.

The term “oxo” refers to the group ═O.

As used herein, the term “haloalkyl” means and alkyl group in which one or more (including all) the hydrogen radicals are replaced by a halo group, wherein each halo group is independently selected from —F, —Cl, —Br, and —I. Representative haloalkyl groups include trifluoromethyl, bromomethyl, 1,2-dichloroethyl, 4-iodobutyl, 2-fluoropentyl, and the like.

“Alkoxy” means an alkyl radical attached through an oxygen linking atom, represented by —O-alkyl. For example, “(C₁-C₄)alkoxy” includes methoxy, ethoxy, proproxy, and butoxy.

“Haloalkoxy” is a haloalkyl group which is attached to another moiety via an oxygen atom such as, e.g., —OCHF₂ or —OCF₃.

The term “aryl” used alone or as part of a larger moiety as in “aralkyl”, “aralkoxy”, or “aryloxyalkyl”, refers to monocyclic and bicyclic carbon ring systems having a total of six to 10 ring members, wherein at least one ring in the system is aromatic. Examples include, but are not limited to phenyl, naphthyl, anthracyl and the like. It will be understood that when specified, optional substituents on an aryl group may be present on any substitutable position.

As used herein, the term “heterocyclyl” means a 4- to 12-membered monocyclic or polycyclic saturated or partially unsaturated heterocyclic ring (e.g., bridged bicyclic) containing 1 to 4 heteroatoms independently selected from N, O, and S. The heterocycle may be attached via any heteroatom or carbon atom, as valency permits. Representative heterocycles include morpholinyl, thiomorpholinyl, pyrrolidinonyl, pyrrolidinyl, piperidinyl, piperazinyl, oxiranyl, dioxanyl, oxetanyl, dihydrofuranyl, dihydropyranyl, isoindolinyl, dihydropyridinyl, tetrahydrofuranyl, tetrahydropyranyl, tetrahydropyrindinyl, tetrahydropyrimidinyl, diazabicyclooctanyl, hexahydropyrrolizinyl, and the like. Optional substituents on a heterocyclyl group may be present on any substitutable position and, include, e.g., the position at which the heterocyclyl is attached.

As used herein, the term “heteroaryl” means a 5- to 12-membered aromatic radical containing 1-4 heteroatoms selected from N, O, and S. A heteroaryl group may be mono- or bicyclic. The heteroaryl may be attached via any heteroatom or carbon atom, as valency permits. Representative heteroaryl groups include pyridyl, furanyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, benzothienyl, and the like. Optional substituents on a heteroaryl group may be present on any substitutable position and, include, e.g., the position at which the heteroaryl is attached.

As used herein, the term “halogen” or “halo” means F, Cl, Br or I.

The term “linker” or “tether,” used interchangeably, refers to a chemical moiety that joins two other moieties (e.g., a first binding moiety and a second binding moiety). A linker can covalently join a first binding moiety and a second binding moiety. In one aspect, the linker is uncleavable in vivo. In one aspect, the linker comprises one or more cyclic ring systems. In another aspect, the linker comprises an alkyl chain optionally substituted by and/or interrupted with one or more chemical groups. In one aspect, the linker comprises optimal spatial and chemical properties to effectuate optimal therapeutic activity. In one aspect, the linker does not interfere with the ability of the first binding moiety and/or the second binding moiety to bind their respective targets (e.g., CRBN or VHL and KRAS(G12D)). In one aspect, the linker alters the ability of the first binding moiety and/or the second binding moiety to bind their respective targets (e.g., CRBN or VHL and KRAS(G12D)).

The term “KRAS” refers to the protein product of the KRAS proto-oncogene, GTPase gene.

The term “KRAS(G12D)” refers to the protein product of the KRAS gene carrying a mutation that results in the glycine amino acid at position 12 of KRAS being replaced by a aspartate.

The term “CRBN” refers to the E3-ubiquitin ligase also known as cereblon, MRT2 and MRT2A along with all its isoforms and splice variants.

The term “VHL” refers to the E3-ubiquitin ligase also known as RCA1, VHL1, pVHL, and HRCA along with all of its isoforms and splice variants.

When used in connection to describe a chemical group that may have multiple points of attachment, a hyphen (—) designates the point of attachment of that group to the variable to which it is defined. For example, —NH(C₁-C₄)alkyl means that the point of attachment for this group occurs on the nitrogen atom.

A hash bond as in “ [symbol] ” represents the point at which the depicted group is attached to the defined variable.

The compounds described herein may have chiral centers and/or geometric centers (E- and Z-isomers). It will be understood that the present disclosure encompasses all stereoisomers and geometric isomers. Tautomeric forms of the compounds described herein are also part of the present disclosure.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the depicted stereoisomer plus the weight of the other stereoisomers.

For use in medicines, the pharmaceutically acceptable salts of the disclosed compounds refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) of the state of disease, amelioration or palliation of the disease state, diminishing rate of or time to progression, and remission (whether partial or total). “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations. In one aspect, a therapeutically effective amount refers to a dosage of from about 0.01 to about 100 mg/kg body weight/day.

The terms “administer,” “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. In another preferred embodiment, an agent is administered orally. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

### 3. Compounds

In a first embodiment, the compound of Formula I is of the Formula II:

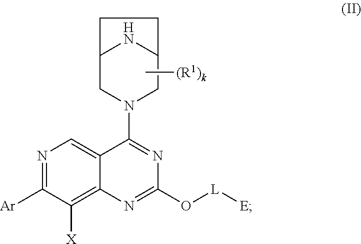

or a pharmaceutically acceptable salt thereof, wherein R¹ is halo, (C₁-C₄)alkyl, hydroxy(C₁-C₄)alkyl, cyano(C₁-C₄)alkyl, CH(═O)—, C(O)₂H, —C(O)₂(C₁-C₄)alkyl, C(O)₂NH₂, —C(O)₂NH(C₁-C₄)alkyl, —C(O)₂N[(C₁-C₄)alkyl]₂, or a 5- to 6-membered optionally substituted heteroaryl; k is 0, 1, 2, or 3; and the remaining variables are as described above for Formula I.

In a second embodiment, the compound of Formula I is of the Formula III:

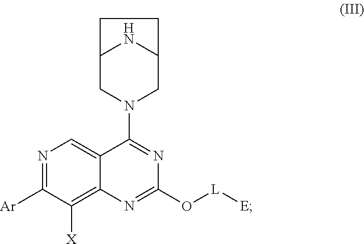

or a pharmaceutically acceptable salt thereof, wherein the remaining variables are as described above for Formula I.

In a third embodiment, X in the compound of Formula I, II, or III, or a pharmaceutically acceptable salt thereof, is hydrogen or fluoro, wherein the remaining variables are as described above for Formula I or Formula II. Alternatively, as part of a third embodiment, X in the compound of Formula I, II, or III, or a pharmaceutically acceptable salt thereof, is fluoro, wherein the remaining variables are as described above for Formula I or Formula II.

In a fourth embodiment, Ar in the compound of Formula I, II, or III, or a pharmaceutically acceptable salt thereof, is an optionally substituted phenyl or optionally substituted naphthalenyl, wherein the remaining variables are as described above for Formula I, Formula II, or the third embodiment. Alternatively, as part of a fourth embodiment, Ar in the compound of Formula I, II, or III, or a pharmaceutically acceptable salt thereof, is phenyl or naphthalenyl, each of which are optionally substituted with one to three groups independently selected from R^{A}, wherein R^{A} is selected from halo, (C₁-C₄)alkyl, (C₂-C₄)alkynyl, (C₂-C₄)alkynylNH₂, (C₁-C₄)alkoxy, halo(C₁-C₄)alkoxy, (C₁-C₄)alkylOH, OH, NH₂, —NH(C₁-C₄)alkyl, —N[(C₁-C₄)alkyl]₂, C(O)NH₂, C(O)NH(C₁-C₄)alkyl, C(O)[(C₁-C₄)alkyl]₂, —NHC(O)(C₁-C₄)alkyl, —N(C₁-C₄)alkylC(O)(C₁-C₄)alkyl, —NHC(O)O(C₁-C₄)alkyl, NHC(O)NH(C₁-C₄)alkyl, CN, —S(C₁-C₄)alkyl, —Shalo(C₁-C₄)alkyl, and (C₃-C₆)cycloalkyl, wherein said (C₁-C₄)alkyl and said (C₃-C₆)cycloalkyl are each optionally substituted with one to two groups selected from halo, (C₁-C₄)alkoxy, halo(C₁-C₄)alkoxy, OH, NH₂, —NH(C₁-C₄)alkyl, —N[(C₁-C₄)alkyl]₂, C(O)NH₂, C(O)NH(C₁-C₄)alkyl, C(O)[(C₁-C₄)alkyl]₂, —NHC(O)(C₁-C₄)alkyl, —N(C₁-C₄)alkylC(O)(C₁-C₄)alkyl, —NHC(O)O(C₁-C₄)alkyl, NHC(O)NH(C₁-C₄)alkyl, CN, —S(C₁-C₄)alkyl, and —Shalo(C₁-C₄)alkyl, and wherein the remaining variables are as described above for Formula I, Formula II, or the third embodiment. In another alternative, as part of a fourth embodiment, Ar in the compound of Formula I, II, or III, or a pharmaceutically acceptable salt thereof, is naphthalenyl optionally substituted with one to three groups independently selected from R^{A}, wherein R^{A} is selected from halo, (C₁-C₄)alkyl, (C₂-C₄)alkynyl, (C₂-C₄)alkynylNH₂, (C₁-C₄)alkoxy, halo(C₁-C₄)alkoxy, (C₁-C₄)alkylOH, OH, NH₂, —NH(C₁-C₄)alkyl, —N[(C₁-C₄)alkyl]₂, C(O)NH₂, C(O)NH(C₁-C₄)alkyl, C(O)[(C₁-C₄)alkyl]₂, —NHC(O)(C₁-C₄)alkyl, —N(C₁-C₄)alkylC(O)(C₁-C₄)alkyl, —NHC(O)O(C₁-C₄)alkyl, NHC(O)NH(C₁-C₄)alkyl, CN, —S(C₁-C₄)alkyl, —Shalo(C₁-C₄)alkyl, and (C₃-C₆)cycloalkyl, wherein said (C₁-C₄)alkyl and said (C₃-C₆)cycloalkyl are each optionally substituted with one to two groups selected from halo, (C₁-C₄)alkoxy, halo(C₁-C₄)alkoxy, OH, NH₂, —NH(C₁-C₄)alkyl, —N[(C₁-C₄)alkyl]₂, C(O)NH₂, C(O)NH(C₁-C₄)alkyl, C(O)[(C₁-C₄)alkyl]₂, —NHC(O)(C₁-C₄)alkyl, —N(C₁-C₄)alkylC(O)(C₁-C₄)alkyl, —NHC(O)O(C₁-C₄)alkyl, NHC(O)NH(C₁-C₄)alkyl, CN, —S(C₁-C₄)alkyl, and —Shalo(C₁-C₄)alkyl, and wherein the remaining variables are as described above for Formula I, Formula II, or the third embodiment. In another alternative, as part of a fourth embodiment, Ar in the compound of Formula I, II, or III, or a pharmaceutically acceptable salt thereof, is phenyl or naphthalenyl, each of which are optionally substituted with one to three groups independently selected from R^{A}, wherein R^{A} is selected from (C₂-C₄)alkynyl, halo, and OH, and wherein the remaining variables are as described above for Formula I, Formula II, or the third embodiment. In another alternative, as part of a fourth embodiment, Ar in the compound of Formula I, II, or III, or a pharmaceutically acceptable salt thereof, is naphthalenyl optionally substituted with one to three groups independently selected from R^{A}, wherein R^{A} is selected from (C₂-C₄)alkynyl, halo, and OH, and wherein the remaining variables are as described above for Formula I, Formula II, or the third embodiment. In another alternative, as part of a fourth embodiment, Ar in the compound of Formula I, II, or III, or a pharmaceutically acceptable salt thereof, is of the structural formula:

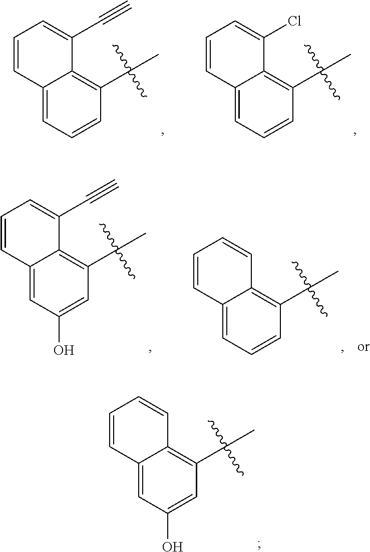 [SMILES: *c1cccc2cccc(C#C)c12 ; *c1cccc2cccc(Cl)c12 ; *c1cc(O)cc2cccc(C#C)c12 ; *c1cccc2ccccc12 ; *c1cc(O)cc2ccccc12]

or a pharmaceutically acceptable salt thereof, wherein the remaining variables are as described above for Formula I, Formula II, or the third embodiment.

In a fifth embodiment, E in the compound of Formula I, II, or III, or a pharmaceutically acceptable salt thereof, is selected from a cereblon (CRBN) modulator and a von Hippel-Lindau (VHL) ligand, wherein the remaining variables are as described above for Formula I, Formula II, or the third embodiment or fourth embodiment. Alternatively, as part of a fifth embodiment, E in the compound of Formula I, II, or III, or a pharmaceutically acceptable salt thereof, is of the structural formula:

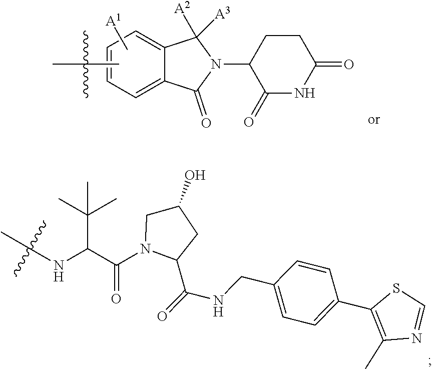

wherein A¹ is halo; and A² and A³ are both hydrogen or A² and A³ taken together form ═O, and wherein the remaining variables are as described above for Formula I, Formula II, or the third embodiment or fourth embodiment. In another alternative, part of a fifth embodiment, E in the compound of Formula I, II, or III, or a pharmaceutically acceptable salt thereof, is of the structural formula:

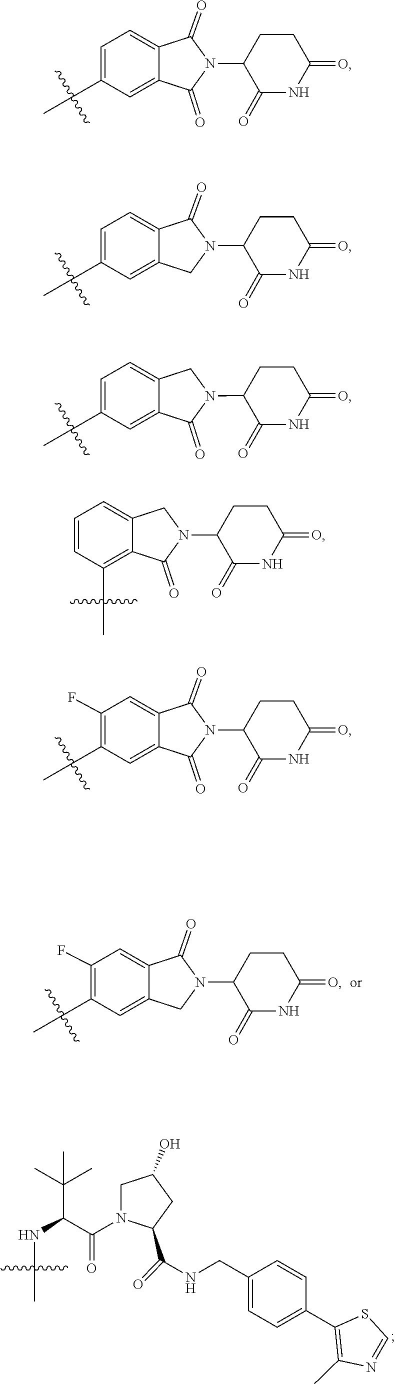

wherein A¹ is halo; and A² and A³ are both hydrogen or A² and A³ taken together form ═O, and wherein the remaining variables are as described above for Formula I, Formula II, or the third embodiment or fourth embodiment.

In a sixth embodiment, L in the compound of Formula I, II, or III, or a pharmaceutically acceptable salt thereof, is -Het¹-X¹-*, *-Het¹-Het²-X¹—, *-NR^{c}—X¹-Het¹-Het²-X²—, *-X¹-Het¹-X²-Het²-(CH₂)ₘO—, —NR^{c}—(CH₂)ₘ—X¹-Het¹-X²—*, —NR^{c}—(CH₂)ₘ—X¹—NR^{c}—(CH₂CH₂O)ₙ—*, —NR^{c}—(CH₂)ₘ—X¹—NR^{c}—(CH₂)ₚ—*, —NR^{c}—(CH₂)ₘ—X¹-Het¹-X²-Het²-X³—*, —O(CH₂)ₘ—X¹-Het¹-X²-Het²-X³—*, —O(CH₂)ₘ—X¹—NR^{C}—(CH₂)ₚ-Het¹-X²-Het²-X³-*, *-X¹—NR^{c}—(CH₂)ₘ-Het¹-X²-Het²-X³—(CH₂)ₚ—NR^{d}—(CH₂)ₚ—, —NR^{C}—(CH₂)ₘ—X¹—(CH)CH₃—Het¹-X²- Het³-X³-*, —NR^{c}—(CH₂)ₘ—X¹—(CH₂)ₚ-Het¹-X²-Het²-X³-*, —NR^{c}—(CH₂)ₘ—X¹—NR^{d}—(CH₂)ₚ- Het¹-X²-Het²-X³-*, —NR^{c}—(CH₂)ₘ—NR^{d}—X¹-Het¹-X²-*, *Het¹-X¹-Het²-X²—, *X¹-Het¹-X²-*(CH₂CH₂O)ₙ—NR^{c}—X¹-Het¹-Het²-X²—, *-Het¹-X¹-Het²-X²-Het³-X³—, *X¹-Het-X²-Het²-X³—, *-Het¹-X¹—NR-Het²-X²—, *X¹-Het¹-Het²-X²—, *X¹—(CH₂)ₘO-Het-X²—, *X¹—(CH₂)ₘNR^{c}—X²-Het¹-Het²-X²—, *-Het¹-X¹-Het²-X²—O—, *-O(CH₂)ₘ Het¹-(CH₂)ₚ—O(CH₂)ₘ—NR^{c}—X²—*, *-Het¹-O—(CH₂)ₘ—X¹-Het²-X²—, *-Het¹-O—(CH₂)ₘ—X¹—NR—(CH₂CH₂O)ₙ(CH₂)ₘ-Het²-X²—, *-Het¹-X¹—NR^{C}—(CH₂)ₘ—, *-Het¹-X¹-Het²-Het³-X²—, *-Het¹-X¹—NR^{c}—(CH₂CH₂O)ₙ(CH₂)ₘ—, *-Het¹-X¹—NR^{c}—(CH₂CH₂O)ₙHet²-(CH₂)ₘ—X²—, *-Het¹-X¹—NR^{c}—(CH₂CH₂O)ₙ—, *-Het¹-X¹—NR^{c}—(CH₂)ₘ-Het²-X²-Het³-(CH₂)ₘ—, *-Het¹-X¹-Het²-(CH₂)ₘ-Het³-X²—, *-Het¹-X¹-Het²-, *-Het¹-X¹—NR^{c}—, *-Het¹-X¹—NR^{c}—(CH₂)ₘ-Phe-X²-Het²-(CH₂)ₘ—, *-Het¹-X¹-Het²-Het³-, *-Het¹-X¹-Het²-(CH₂)ₘ Het³-X²—(CH₂)ₚ—NR^{c}—(CH₂)ₘ—, *-Het¹-X¹-Het²-(CH₂)ₘ-Het³-(CH₂)ₘ—O—, *- Het¹-X¹-Het²-(CH₂)ₘ Het³-(CH₂)ₚ—NR^{c}—(CH₂)ₘ—, *-Het¹-X¹-Het²-(CH₂CH₂O)ₙ—,*-Het¹-X¹—(CH₂)ₘ-Het²-X²—, *—(CH₂CH₂O)ₒ—(CH₂)ₚ-Het¹-X¹-Het²-(CH₂CH₂O)ₙ, *-(CH₂CH₂O)ₙ—(CH₂)ₘ-Het¹-X¹-Het²-X², *-Het¹-X¹-Phe-X²—NR^{c}—X³—, *-(CH₂CH₂O)ₒ—(CH₂)ₚ-Het¹-X¹-Phe-X²—NR^{c}—(CH₂CH₂O)ₙ—, *-(CH₂CH₂O)ₙ—(CH₂)ₘ—NR^{c}-Phe-X¹—, *-(CH₂CH₂O)ₒ—(CH₂)ₚ—NR^{c}-Phe- (CH₂CH₂O)ₙ—, *-(CH₂CH₂O)ₒ—(CH₂)ₚ—NR^{c}—(CH₂CH₂O)ₙ—(CH₂)ₘ—, *-(CH₂CH₂O)ₙ—(CH₂)ₘ—NR^{c}—(CH₂CH₂O)ₙ—(CH₂)ₘ—C(O)—NR^{d}—(CH₂CH₂O)ₒ—(CH₂)ₚ—, *-(CH₂CH₂O)ₒ—(CH₂)ₚ—NR^{c}—(CH₂CH₂)ₙ—(CH₂)ₘ-Het¹-X¹-Het²-X²—, *-(CH₂CH₂O)ₒ—(CH₂)ₚ—NR^{c}—(CH₂CH₂O)ₙ—(CH₂)ₘ-Het¹-X¹-Het²-X²—(CH₂CH₂O)ₒ, *-NR^{c}—(CH₂CH₂O)ₙ—(CH₂)ₘ-Phe-NH—X¹-Het¹-X², *-NR^{c}—(CH₂CH₂O)ₙ—(CH₂)ₘ-Phe-NH—X¹-Het¹-X²—(CH₂CH₂O)ₒ, *-(CH₂CH₂O)ₒ—(CH₂)ₚ—NR^{c}—(CH₂CHO₂O)ₙ—(CH₂)ₘ-Phe-X¹—NR^{C}—(CH₂CH₂O)ₒ—(CH₂)ₚ—, *-(CH₂CH₂O)ₒ—(CH₂)ₚ—NR^{c}—(CH₂CH₂O)ₙ—(CH₂)ₘ-Het¹-X¹—, *-(CH₂CH₂O)ₒ—(CH₂)ₚ—NR^{c}—(CH₂CH₂O)ₙ—(CH₂)ₘ-Het¹- X¹—(CH₂CH₂O)ₙ—, *-(CH₂CH₂O)ₙ—(CH₂)ₘ—NR^{c}—(CH₂)ₘ—C(O)—NR^{d}-Het¹-X¹-Het²-(CH₂CH₂O)ₒ—(CH₂)ₚ, or *-NR^{c}—(CH₂)ₘ—C(O)—NR^{d}—(CH₂)ₘ-Het¹-X¹-Het²-X²—;
* indicates the point of attachment to E;
Het¹, Het², and Het³ are each independently phenyl, a 5- to 8-membered heterocyclyl, 5- to 7-membered heteroaryl, or a 3- to 6-membered cycloalkyl, each of which are optionally substituted with (C₁-C₄)alkyl;
Phe is phenyl;
X¹, X², and X³, are each independently C(O) or (CH₂)ᵣ;
R^{c} and R^{d} are each independently hydrogen or (C₁-C₄)alkyl; and
m, n, o, p, and r are each independently integers selected from 0, 1, 2, 3, 4, 5, and 6, and wherein the remaining variables are as described above for Formula I, Formula II, or the third embodiment, fourth, or fifth embodiment. Alternatively, as part of a sixth embodiment, L in the compound of Formula I, II, or III, or a pharmaceutically acceptable salt thereof, is *-Het¹-Het²-X¹—, *-NR^{c}—X¹-Het¹-Het²-X²—, *Het¹-X¹-Het²-X²—, *X¹-Het¹-X²—, *(CH₂CH₂O)ₙ—NR^{c}—X¹-Het¹-Het²-X²—, *Het¹-X¹-Het²-X²-Het³-X³—, *X¹-Het¹-X²-Het²-X³—, *-Het¹-X¹—NR^{c}-Het²-X²—, *X¹-Het¹-Het²-X²—, *X¹—(CH₂)ₘO-Het-X²—, *X¹—(CH₂)ₘNR^{c}—X²-Het¹-Het²-X²—, or *-Het¹-X¹-Het²-Het³-X²,
* indicates the point of attachment to E;
Het¹, Het², and Het³ are each independently phenyl, a 5- to 8-membered heterocyclyl, 5- to 7-membered heteroaryl, or a 3- to 6-membered cycloalkyl, each of which are optionally substituted with (C₁-C₄)alkyl;
X¹, X², and X³, are each independently C(O) or (CH₂)ᵣ;
R^{c} is hydrogen or (C₁-C₄)alkyl; and
m, n, and r are each independently integers selected from 0, 1, 2, 3, 4, 5, and 6 and wherein the remaining variables are as described above for Formula I, Formula II, or the third embodiment, fourth, or fifth embodiment.

In a seventh embodiment, Het¹, Het², and Het³ as defined above are each independently a 5- to 8-membered heterocyclyl, or a 3- to 6-membered cycloalkyl, each of which are optionally substituted with (C₁-C₄)alkyl, wherein the remaining variables are as described above for Formula I, Formula II, or the third embodiment, fourth, fifth, or sixth embodiment.

In an eighth embodiment, m, n, o, p, and r as defined above are each independently integers selected from 0, 1, 2, or 3, wherein the remaining variables are as described above for Formula I, Formula II, or the third embodiment, fourth, fifth, sixth, or seventh embodiment.

In a ninth embodiment, L in the compound of Formula I, II, or III, or a pharmaceutically acceptable salt thereof, is selected from

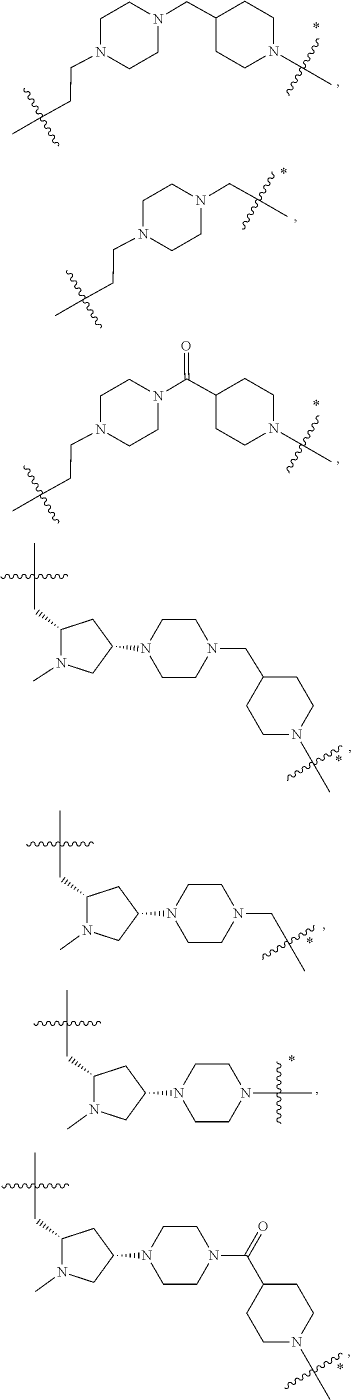

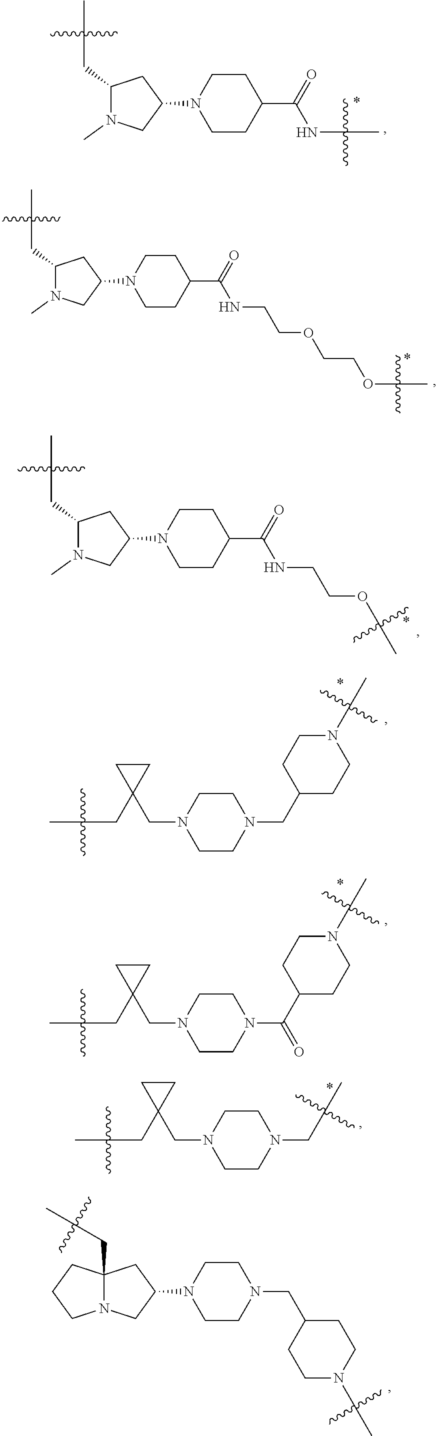

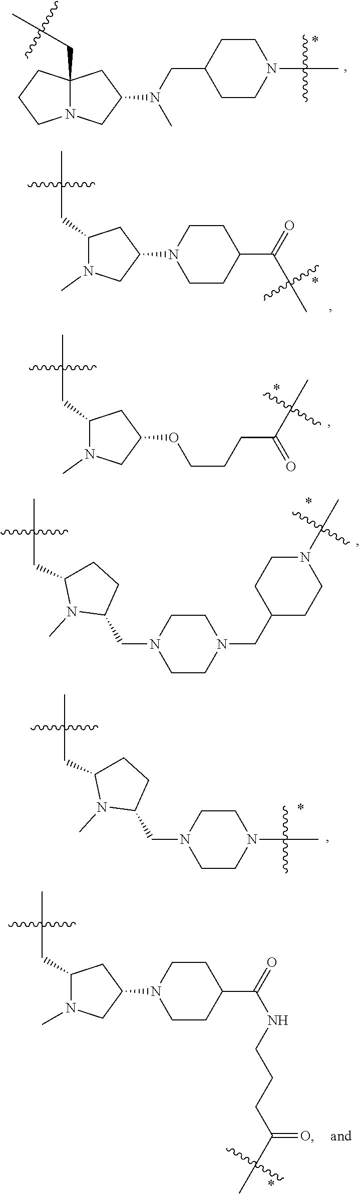 [SMILES: *C[C@]12CCCN1C[C@@H](N(C)CC1CCN(*)CC1)C2 ; *C[C@@H]1C[C@H](N2CCC(C(*)=O)CC2)CN1C ; *C[C@@H]1C[C@H](OCCCC(*)=O)CN1C ; *C[C@@H]1CC[C@H](CN2CCN(CC3CCN(*)CC3)CC2)N1C ; *C[C@@H]1CC[C@H](CN2CCN(*)CC2)N1C ; *C[C@@H]1C[C@H](N2CCC(C(=O)NCCCC(*)=O)CC2)CN1C]

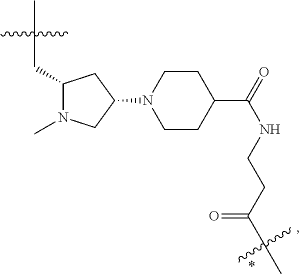 [SMILES: *C[C@@H]1C[C@H](N2CCC(C(=O)NCCC(*)=O)CC2)CN1C]

wherein the remaining variables are as described above for Formula I, Formula II, or the third embodiment, fourth, or fifth, embodiment.

Compounds having the Formula I, II, or III are further disclosed in the Exemplification and are included in the present disclosure. Pharmaceutically acceptable salts thereof as well as the neutral forms are included.

### 4. Uses, Formulation and Administration

Compounds and compositions described herein are generally useful as anticancer therapies. In one aspect, the disclosed compounds and compositions behave as proteolysis-targeting chimeras (PROTACs) in which one portion of the compounds is responsible for binding KRAS(G12D) and the other portion is responsible for binding to E3 ubiquitin-ligases, CRBN or VHL. Their mechanisms of action include, but are not limited to, degrading KRAS(G12D) and thereby impeding down-stream signals that may result in inhibition of cancer cell growth and/or induction of cancer cell death or other KRAS or KRAS(G12D) functions.

In one aspect, the disclosed compounds effectuate the degradation of KRAS(G12D).

In one aspect, the disclosed compounds may effectuate the activity of KRAS(G12D).

In one aspect, the disclosed compounds may effectuate the protein-protein interactions between KRAS(G12D) and upstream signaling component such as SOS1.

In one aspect, the disclosed compounds may effectuate the protein-protein interactions between KRAS(G12D) and downstream signaling components such as RAF1 or PI3K. Thus, provided herein are methods of treating conditions which are responsive to the degradation of KRAS(G12D) comprising administering to a subject in need thereof, a therapeutically effective amount of one or more compounds or compositions described herein. Also provided is the use of one or more compounds or compositions described herein in the manufacture of a medicament for treating conditions which are responsive to the degradation of KRAS(G12D). Further provided is the use of a compound or composition described herein for treating conditions which are responsive to the degradation of KRAS(G12D).

In one aspect, the condition treated by the present compounds and compositions is a cancer. The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin and non-Hodgkin), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” or “tumorous tissue” are understood as cells, extracellular matrix, and other naturally occurring components associated with the solid tumor.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### EXEMPLIFICATION

### Chemical Synthesis

The representative examples that follow are intended to help illustrate the present disclosure, and are not intended to, nor should they be construed to, limit the scope of the invention. General starting materials used were obtained from commercial sources or prepared in other examples, unless otherwise noted.

### Preparation of Compounds

The compounds claimed herein were prepared following the procedures outlined in the following protocols.

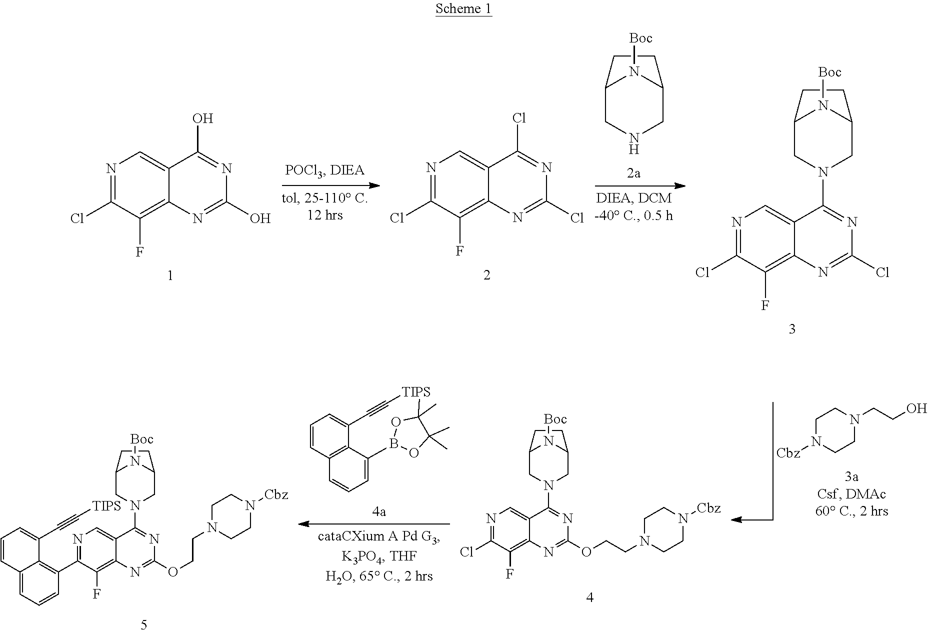

### Intermediate 2:

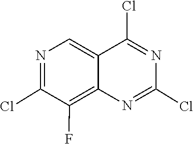 [SMILES: Fc1c(Cl)ncc2c(Cl)nc(Cl)nc12]

### 2,4,7-trichloro-8-fluoropyrido[4,3-d]pyrimidine

2 batches: To a mixture of compound 1 (50.0 g, 232 mmol, 1.00 eq) in Tol. (150 mL) was added POCl₃ (178 g, 1.16 mol, 108 mL, 5.00 eq) at 25° C. Then DIEA (65.9 g, 510 mmol, 88.9 mL, 2.20 eq) was added into the mixture blow 40° C. The mixture was stirred at 110° C. for 12 hrs. LCMS showed desired MS was detected. The reaction mixture was distilled under reduced pressure to remove POCl₃ at 90° C. The residue was poured into Sat·NaHCO₃ slowly (keep pH=8). During this period, yellow precipitate was formed. It was collected by filtration and washed by H₂O. The solid was used for next step directly. Compound 2 (101 g, 400 mmol, 86.2% yield) was obtained as brown solid. HNMR (DMSO-d₆, 400 MHz): δ 8.92-8.86 (m, 1H). LC-MS: m/z 253.9 [M+H]⁺.

### Intermediate 3:

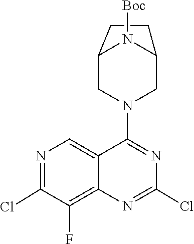 [SMILES: CC(C)(C)OC(=O)N1C2CCC1CN(c1nc(Cl)nc3c(F)c(Cl)ncc13)C2]

### tert-butyl 3-(2,7-dichloro-8-fluoropyrido[4,3-d]pyrimidin-4-yl)-3,8-diazabicyclo[3.2.1]octane-8-carboxylate

2 batches: To a mixture of compound 2 (48.5 g, 192 mmol, 1.00 eq) in DCM (485 mL) was added a solution of compound 2a (38.7 g, 183 mmol, 0.950 eq) in DCM (120 mL). Then DIEA (49.7 g, 384 mmol, 66.9 mL, 2.00 eq) was added into the mixture at −40° C. and stirred at −40° C. for 0.5 hr under N₂. LCMS showed compound 2 was consumed, and desired MS was detected. The mixture was quenched by HCl (0.5 M) and the pH of the aqueous phase was acidified to 6-7, then separated, the organic layer was dried over Na₂SO₄. The residue was purified by column chromatography (SiO₂, TLC:Petroleum ether:Ethyl acetate=3:1, R_{f}=0.4, Petroleum ether:Ethyl acetate=10:1 to 1:1, R_{f}=0.4). Compound 3 (120 g, 280 mmol, 72.9% yield) was obtained as white solid. HNMR (DMSO-d₆, 400 MHz): δ 9.13-8.98 (m, 1H), 4.67-4.36 (m, 2H), 4.35-4.21 (m, 2H), 3.87-3.50 (m, 2H), 1.85-1.71 (m, 2H), 1.66-1.56 (m, 2H), 1.46 (s, 9H). LC-MS: m/z 428.0[M+H]⁺.

### Intermediate 4:

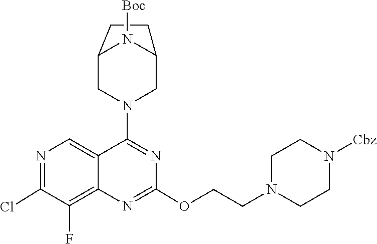 [SMILES: CC(C)(C)OC(=O)N1C2CCC1CN(c1nc(OCCN3CCN(C(=O)OCc4ccccc4)CC3)nc3c(F)c(Cl)ncc13)C2]

### tert-butyl 3-(2-(2-(4-((benzyloxy)carbonyl)piperazin-1-yl)ethoxy)-7-chloro-8-fluoropyrido[4,3-d]pyrimidin-4-yl)-3,8-diazabicyclo[3.2.1]octane-8-carboxylate

To a mixture of compound 3 (10.0 g, 23.4 mmol, 1.00 eq) and compound 3a (9.26 g, 35.0 mmol, 1.50 eq) in DMAc (100 mL) was added CsF (10.6 g, 70.0 mmol, 2.58 mL, 3.00 eq) and stirred at 60° C. for 2 hrs under N₂. LCMS showed compound 3 was consumed, and desired MS was detected. The mixture was added into H₂O (200 mL) and extracted with Ethyl acetate (200 mL*2), washed with sat·NaCl (300 mL), dried over Na₂SO₄, filtered and concentrated under reduced pressure to give oil. The oil was purification by column chromatography (SiO₂, TLC:Petroleum ether:Ethyl acetate=1:1, R_{f}=0.1, Petroleum ether:Ethyl acetate=10:1 to 1:1, R_{f}=0.1). Compound 4 (7.50 g, 11.4 mmol, 49.0% yield) was obtained as white solid. HNMR (CDCl₃, 400 MHz): δ 8.74 (s, 1H), 7.30 (s, 5H), 5.13 (s, 2H), 4.62-4.58 (m, 2H), 4.49-4.45 (m, 2H), 4.41-4.29 (m, 2H), 3.71-3.60 (m, 2H), 3.54-3.50 (m, 4H), 2.87-2.83 (m, 2H), 2.60-2.53 (m, 4H), 1.99-1.93 (m, 2H), 1.74-1.69 (m, 2H), 1.53-1.51 (m, 9H). LC-MS: m/z 656.2[M+H]⁺.

### Intermediate 5:

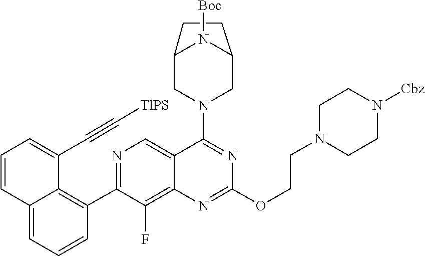

### tert-butyl 3-(2-(2-(4-((benzyloxy)carbonyl)piperazin-1-yl)ethoxy)-8-fluoro-7-(8-((triisopropylsilyl)ethynyl)naphthalen-1-yl)pyrido[4,3-d]pyrimidin-4-yl)-3,8-diazabicyclo[3.2.1]octane-8-carboxylate

To a mixture of compound 4 (12.7 g, 19.4 mmol, 1.00 eq) and compound 4a (12.6 g, 29.0 mmol, 1.50 eq) in THF (127 mL) was added K₃PO₄ (1.50 M, 38.7 mL, 3.00 eq) and cataCXium A Pd G3 (2.11 g, 2.90 mmol, 0.15 eq). The mixture was stirred at 65° C. for 2 hrs under N₂. LCMS showed compound 4 was consumed, and desired MS was detected. The mixture was added into H₂O (100 mL) and extracted with Ethyl acetate (50 mL*2), washed with sat·NaCl (150 mL), dried over Na₂SO₄, filtered and concentrated under reduced pressure to give oil. The oil was purification by silica gel chromatography eluted with Petroleum ether:Ethyl acetate=100:0 to 1:1, Petroleum ether:Ethyl acetate=1:1, R_{f}=0.3. Compound 5 (10.0 g, 10.5 mmol, 54.3% yield, 97.6% purity) was obtained as brown solid. HNMR (CDCl₃, 400 MHz): δ 9.11-9.07 (m, 1H), 7.99-7.90 (m, 2H), 7.84-7.79 (m, 1H), 7.60-7.52 (m, 2H), 7.50-7.44 (m, 1H), 7.40-7.28 (m, 5H), 5.16-5.09 (m, 2H), 4.79-4.70 (m, 1H), 4.70-4.61 (m, 1H), 4.60-4.52 (m, 1H), 4.46-4.32 (m, 2H), 4.24-4.16 (m, 1H), 3.84-3.66 (m, 1H), 3.56-3.41 (m, 5H), 2.94-2.81 (m, 2H), 2.62-2.51 (m, 4H), 2.03-1.96 (m, 4H), 1.55-1.51 (m, 9H), 0.90-0.83 (m, 18H), 0.61 (s, 3H). LC-MS: m/z 928.4[M+H]⁺.

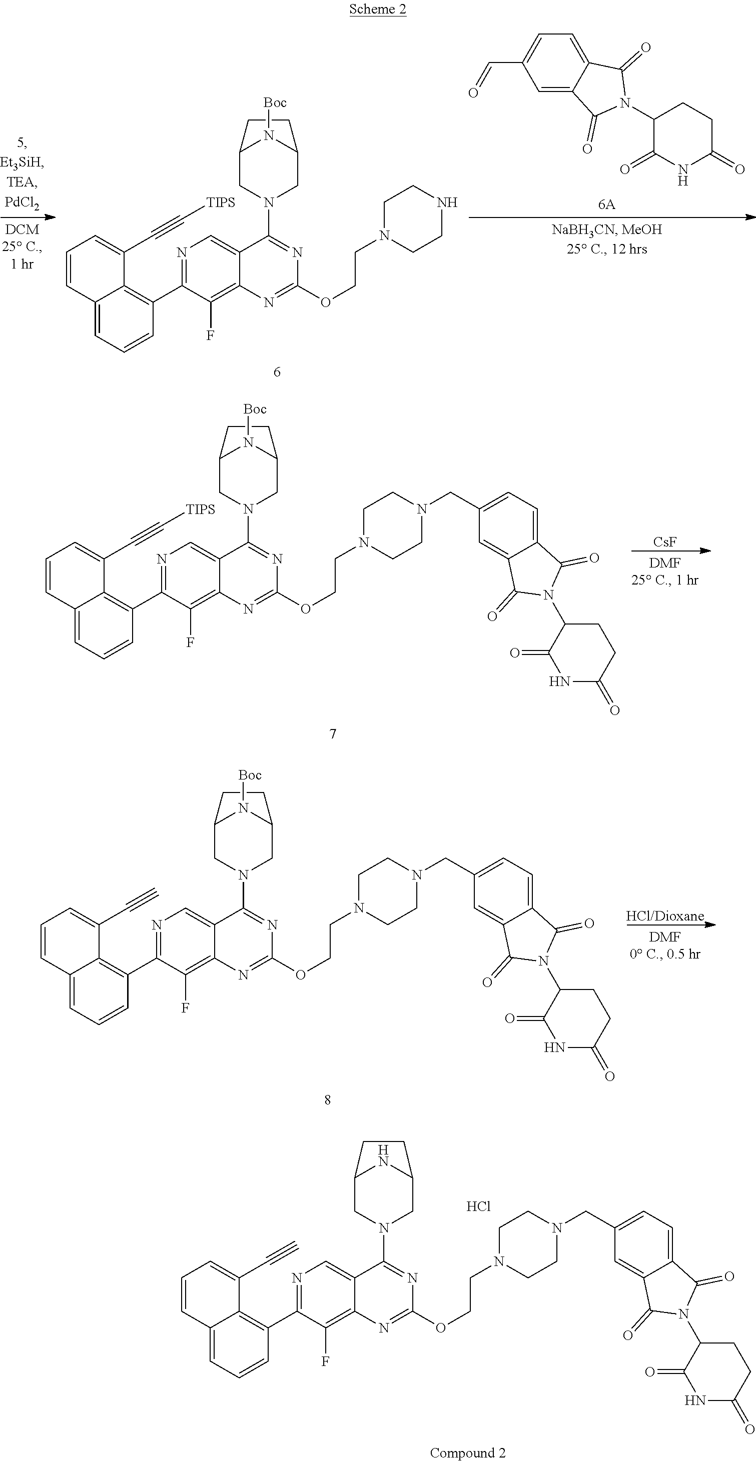

### Intermediate 6:

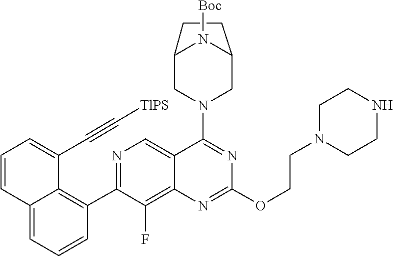

### tert-butyl 3-(8-fluoro-2-(2-(piperazin-1-yl)ethoxy)-7-(8-((triisopropylsilyl)ethynyl)naphthalen-1-yl)pyrido[4,3-d]pyrimidin-4-yl)-3,8-diazabicyclo[3.2.1]octane-8-carboxylate

To a mixture of compound 5 (5.50 g, 5.86 mmol, 98.9% purity, 1.00 eq) in DCM (55.0 mL) was added TEA (4.74 g, 46.9 mmol, 6.53 mL, 8.00 eq), Et₃SiH (4.09 g, 35.2 mmol, 5.62 mL, 6.00 eq) and PdCl₂ (155 mg, 879 umol, 0.15 eq). The mixture was stirred at 25° C. for 1 h. LCMS showed compound 5 was consumed, and desired MS was detected. The mixture was filtered. The solution was added into H₂O (50 mL) and extracted with DCM (40 mL*2), washed with sat·NaCl (100 mL), dried over Na₂SO₄, filtered and concentrated under reduced pressure to give oil. The oil was used for next step directly. Compound 6 (3.30 g, 4.16 mmol, 70.9% yield) was obtained as brown oil. LC-MS: m/z 794.4[M+H]⁺.

### Intermediate 7:

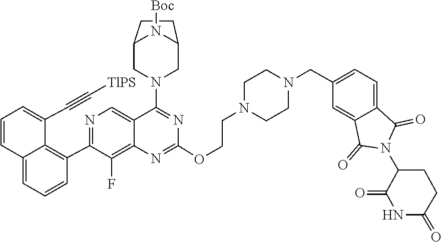

### tert-butyl 3-(2-(2-(4-((2-(2,6-dioxopiperidin-3-yl)-1,3-dioxoisoindolin-5-yl)methyl)piperazin-1-yl)ethoxy)-8-fluoro-7-(8-((triisopropylsilyl)ethynyl)naphthalen-1-yl)pyrido[4,3-d]pyrimidin-4-yl)-3,8-diazabicyclo[3.2.1]octane-8-carboxylate

To a mixture of compound 6 (300 mg, 377 umol, 1.00 eq) and compound 6A (129 mg, 453 umol, 1.20 eq) in MeOH (3.00 mL) was added NaBH₃CN (30.8 mg, 491 umol, 1.30 eq) and stirred at 25° C. for 12 hrs. LCMS showed compound 6 was consumed, and desired MS was detected. The mixture was added into H₂O (8 mL) and extracted with Ethyl acetate (10 mL *2), washed with sat·NaCl (10 mL), dried over Na₂SO₄, filtered and concentrated under reduced pressure to give oil. The oil was purification by reversed-phase HPLC (0.1% TFA condition) to give solid. Compound 7 (400 mg, crude) was obtained as yellow solid. LC-MS: m/z 533.0[M/2+H]⁺.

### Intermediate 8:

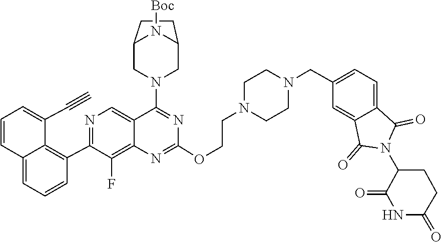 [SMILES: C#Cc1cccc2cccc(-c3ncc4c(N5CC6CCC(C5)N6C(=O)OC(C)(C)C)nc(OCCN5CCN(Cc6ccc7c(c6)C(=O)N(C6CCC(=O)NC6=O)C7=O)CC5)nc4c3F)c12]

### tert-butyl 3-(2-(2-(4-((2-(2,6-dioxopiperidin-3-yl)-1,3-dioxoisoindolin-5-yl)methyl)piperazin-1-yl)ethoxy)-7-(8-ethynylnaphthalen-1-yl)-8-fluoropyrido[4,3-d]pyrimidin-4-yl)-3,8-diazabicyclo[3.2.1]octane-8-carboxylate

To a mixture of compound 7 (400 mg, 375 umol, 1.00 eq) in DMF (4.00 mL) was added CsF (570 mg, 3.76 mmol, 138 μL, 10.0 eq) and stirred at 25° C. for 1 hr. LCMS showed compound 7 was consumed, and desired MS was detected. The mixture was added into H₂O (5 mL) and extracted with Ethyl acetate (5 mL*2), washed with sat·NaCl (5 mL), dried over Na₂SO₄, filtered and concentrated under reduced pressure to give solid. The solid was used for next step directly. Compound 8 (240 mg, 264 umol, 70.3% yield) was obtained as yellow solid. LC-MS: m/z 908.4[M+H]⁺.

### Compound 1:

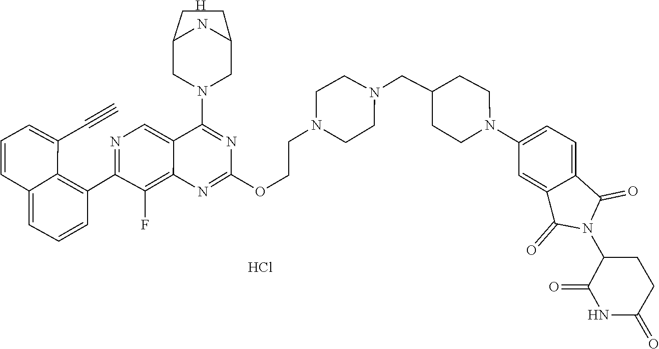

### 5-(4-((4-(2-((4-(3,8-diazabicyclo[3.2.1]octan-3-yl)-7-(8-ethynylnaphthalen-1-yl)-8-fluoropyrido[4,3-d]pyrimidin-2-yl)oxy)ethyl)piperazin-1-yl)methyl)piperidin-1-yl)-2-(2,6-dioxopiperidin-3-yl)isoindoline-1,3-dione hydrochloride

¹H NMR (400 MHz, MeOD): δ 9.16 (s, 1H), 8.21-8.18 (m, 1H), 8.15-8.11 (m, 1H), 7.81-7.78 (m, 1H), 7.76-7.65 (m, 3H), 7.61-7.56 (m, 1H), 7.37 (d, J=2.1 Hz, 1H), 7.25 (dd, J=2.4, 8.5 Hz, 1H), 5.09 (dd, J=5.6, 12.6 Hz, 2H), 5.03-4.90 (m, 6H), 4.36-4.25 (m, 3H), 4.13-3.93 (m, 6H), 3.51-3.47 (m, 3H), 3.15 (td, J=1.6, 3.3 Hz, 1H), 3.07-2.94 (m, 5H), 2.88 (d, J=3.8 Hz, 1H), 2.80-2.77 (m, 1H), 2.75 (d, J=1.8 Hz, 1H), 2.68 (s, 1H), 2.20 (s, 4H), 2.15-2.09 (m, 2H), 1.97-1.90 (m, 2H), 1.46-1.30 (m, 3H). LCMS: m/z 891.8 [M−HCl+H]⁺.

### Compound 2:

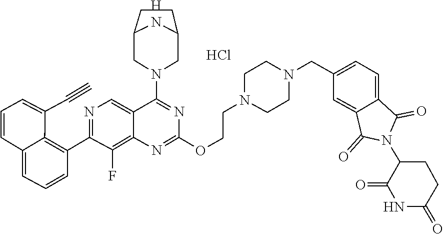

### 5-((4-(2-((4-(3,8-diazabicyclo[3.2.1]octan-3-yl)-7-(8-ethynylnaphthalen-1-yl)-8-fluoropyrido[4,3-d]pyrimidin-2-yl)oxy)ethyl)piperazin-1-yl)methyl)-2-(2,6-dioxopiperidin-3-yl)isoindoline-1,3-dione hydrochloride

To a mixture of compound 8 (120 mg, 132 umol, 1.00 eq) in ACN (1.20 mL) was added HCl/dioxane (4.00 M, 500 μL, 15.1 eq) at 0° C. and stirred at 0° C. for 0.5 hrs. LCMS showed compound 8 was consumed, and desired MS was detected. The mixture was concentrated under reduced pressure at 25° C. to give oil. The oil was purification by prep-HPLC (column: Welch Xtimate C18 150*25 mm*5 um;mobile phase: [water(FA)-ACN];B %: 8%-38%, 10 min). Compound 9 (55.0 mg, 65.9 umol, 49.9% yield, 96.8% purity) was obtained as yellow solid. HNMR (DMSO-d₆, 400 MHz): δ 11.15-11.07 (m, 1H), 9.07-9.02 (m, 1H), 8.17-8.10 (m, 2H), 7.89-7.85 (m, 1H), 7.84-7.81 (m, 1H), 7.81-7.77 (m, 1H), 7.73-7.66 (m, 2H), 7.60-7.53 (m, 2H), 5.18-5.09 (m, 1H), 4.51-4.41 (m, 3H), 4.35-4.28 (m, 1H), 3.72-3.54 (m, 10H), 2.90-2.83 (m, 1H), 2.70 (t, J=5.60 Hz, 2H), 2.64-2.60 (m, 1H), 2.60-2.56 (m, 1H), 2.41 (s, 5H), 2.08-2.01 (m, 1H), 1.73-1.64 (m, 4H). LC-MS: m/z 808.3[M−HCl+H]⁺.

### Compound 3:

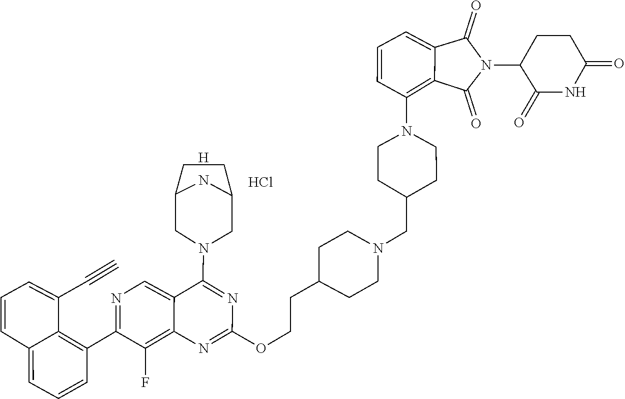

### 4-(4-((4-(2-((4-(3,8-diazabicyclo[3.2.1]octan-3-yl)-7-(8-ethynylnaphthalen-1-yl)-8-fluoropyrido[4,3-d]pyrimidin-2-yl)oxy)ethyl)piperidin-1-yl)methyl)piperidin-1-yl)-2-(2,6-dioxopiperidin-3-yl)isoindoline-1,3-dione hydrochloride

HNMR (DMSO-d₆, 400 HMz): δ 11.12-11.03 (m, 1H), 9.06-9.02 (m, 1H), 8.18-8.10 (m, 2H), 7.75-7.63 (m, 3H), 7.61-7.54 (m, 2H), 7.35-7.26 (m, 2H), 5.13-5.03 (m, 1H), 4.51-4.41 (m, 3H), 4.36-4.28 (m, 1H), 3.72-3.62 (m, 4H), 3.61-3.53 (m, 4H), 2.90-2.81 (m, 3H), 2.74-2.65 (m, 3H), 2.63-2.53 (m, 4H), 2.41-2.29 (m, 5H), 2.19-2.13 (m, 2H), 2.05-1.99 (m, 1H), 1.83-1.75 (m, 2H), 1.71-1.62 (m, 5H), 1.37-1.24 (m, 2H). LC-MS: m/z 891.7 [M−HCl+H]⁺.

### Compound 4:

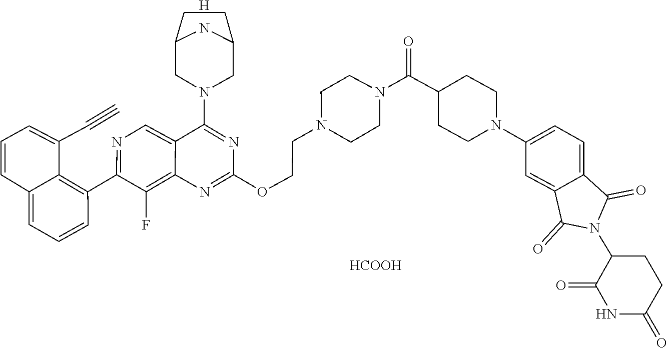

### 5-(4-(4-(2-((4-(3,8-diazabicyclo[3.2.1]octan-3-yl)-7-(8-ethynylnaphthalen-1-yl)-8-fluoropyrido[4,3-d]pyrimidin-2-yl)oxy)ethyl)piperazine-1-carbonyl)piperidin-1-yl)-2-(2,6-dioxopiperidin-3-yl)isoindoline-1,3-dione formate

HNMR (DMSO-d₆, 400 MHz): δ 11.07 (s, 1H), 9.05 (s, 1H), 8.22 (s, 1H), 8.14 (t, J=7.4 Hz, 2H), 7.75-7.63 (m, 3H), 7.61-7.53 (m, 2H), 7.31 (d, J=1.6 Hz, 1H), 7.23 (dd, J=2.0, 8.7 Hz, 1H), 5.06 (dd, J=5.6, 12.9 Hz, 1H), 4.56-4.40 (m, 3H), 4.33 (br d, J=12.4 Hz, 1H), 4.04 (td, J=1.6, 6.6 Hz, 2H), 3.67 (s, 1H), 3.64 (d, J=12.4 Hz, 1H), 3.61-3.51 (m, 6H), 3.10-2.80 (m, 5H), 2.74 (t, J=5.6 Hz, 2H), 2.64-2.54 (m, 2H), 2.52 (d, J=1.6 Hz, 2H), 2.43 (s, 3H), 2.05-1.99 (m, 1H), 1.67 (s, 6H), 1.59 (d, J=12.4 Hz, 2H). LC-MS: m/z 905.3 [M−HCOOH+H]⁺.

### Compound 5:

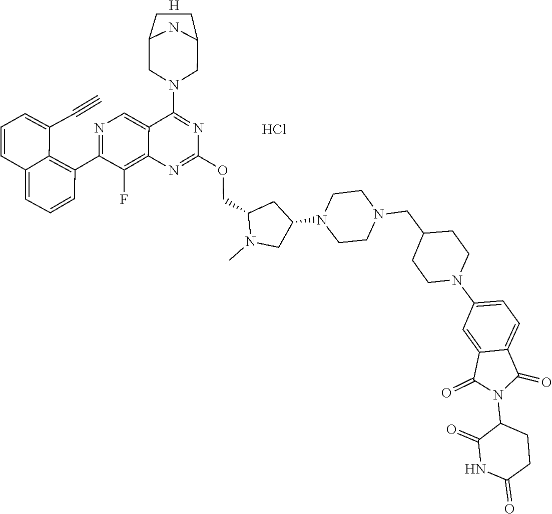

### 5-(4-((4-((3S,5S)-5-(((4-(3,8-diazabicyclo[3.2.1]octan-3-yl)-7-(8-ethynylnaphthalen-1-yl)-8-fluoropyrido[4,3-d]pyrimidin-2-yl)oxy)methyl)-1-methylpyrrolidin-3-yl)piperazin-1-yl)methyl)piperidin-1-yl)-2-(2,6-dioxopiperidin-3-yl)isoindoline-1,3-dione hydrochloride

¹H NMR (400 MHz, CDCl₃): δ 9.05 (s, 1H), 8.24 (s, 2H), 8.02-7.95 (m, 2H), 7.69-7.60 (m, 3H), 7.51-7.44 (m, 1H), 7.03-7.00 (m, 1H), 4.99-4.91 (m, 1H), 4.67-4.53 (m, 5H), 3.92-3.83 (m, 2H), 3.80-3.65 (m, 5H), 3.45-3.31 (m, 2H), 2.92-2.76 (m, 11H), 2.73-2.53 (m, 8H), 2.48 (s, 3H), 2.37-2.24 (m, 3H), 2.18-1.98 (m, 4H), 1.93-1.91 (m, 2H). LC-MS: m/z 960.6 [M−HCl+H]⁺.

### Compound 6:

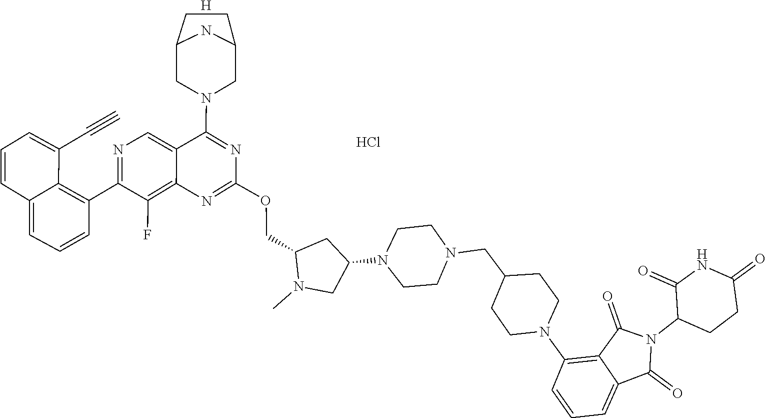

### 4-(4-((4-((3S,5S)-5-(((4-(3,8-diazabicyclo[3.2.1]octan-3-yl)-7-(8-ethynylnaphthalen-1-yl)-8-fluoropyrido[4,3-d]pyrimidin-2-yl)oxy)methyl)-1-methylpyrrolidin-3-yl)piperazin-1-yl)methyl)piperidin-1-yl)-2-(2,6-dioxopiperidin-3-yl)isoindoline-1,3-dione hydrochloride

HNMR (MeOD 400 MHz): δ 9.06 (s, 1H), 8.53 (s, 1H), 8.12-8.02 (m, 2H), 7.77 (d, J=6.6 Hz, 1H), 7.71-7.63 (m, 2H), 7.63-7.59 (m, 1H), 7.53 (J=3.5, 7.7 Hz, 1H), 7.37 (d, J=7.3 Hz, 1H), 7.27 (dd, J=8.5, 15.4 Hz, 1H), 5.16-5.06 (m, 1H), 4.78-4.52 (m, 5H), 3.84-3.67 (m, 6H), 3.13 (d, J=7.9 Hz, 1H), 3.03-2.64 (m, 12H), 2.54 (d, J=4.3 Hz, 3H), 2.42-2.30 (m, 3H), 2.18-2.07 (m, 1H), 2.00-1.73 (m, 8H), 1.54-1.30 (m, 3H). LC-MS: m/z 960.6 [M−HCl+H]⁺.

### Compound 7:

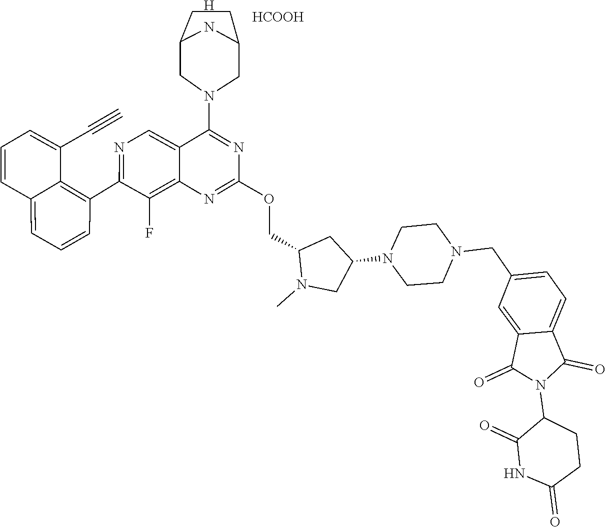

### 5-((4-((3S,5S)-5-(((4-(3,8-diazabicyclo[3.2.1]octan-3-yl)-7-(8-ethynylnaphthalen-1-yl)-8-fluoropyrido[4,3-d]pyrimidin-2-yl)oxy)methyl)-1-methylpyrrolidin-3-yl)piperazin-1-yl)methyl)-2-(2,6-dioxopiperidin-3-yl)isoindoline-1,3-dione formate

HNMR (DMSO-d₆, 400 MHz): δ 11.12 (s, 1H), 9.10 (s, 1H), 8.23-8.11 (m, 2H), 7.88 (d, J=7.5 Hz, 1H), 7.85-7.77 (m, 2H), 7.77-7.68 (m, 2H), 7.62-7.57 (m, 1H), 5.15 (dd, J=5.3, 12.7 Hz, 1H), 4.62 (dd, J=1.8, 11.8 Hz, 1H), 4.50-4.28 (m, 3H), 4.02 (s, 2H), 3.84-3.73 (m, 3H), 3.71-3.62 (m, 5H), 3.08 (dd, J=2.8, 10.6 Hz, 2H), 2.92-2.84 (m, 1H), 2.70-2.66 (m, 1H), 2.65-2.61 (m, 1H), 2.59 (s, 2H), 2.44 (d, J=4.6 Hz, 5H), 2.38-2.33 (m, 4H), 2.21-2.12 (m, 1H), 2.11-2.01 (m, 2H), 1.88 (s, 4H). LCMS: m/z 877.3 [M−HCOOH+H]⁺.

### Compound 8:

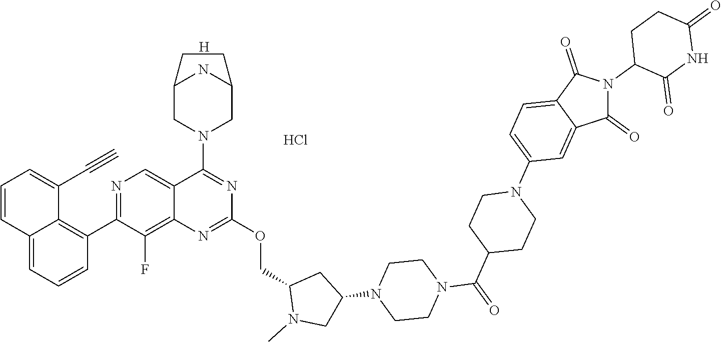

### 5-(4-(4-((3S,5S)-5-(((4-(3,8-diazabicyclo[3.2.1]octan-3-yl)-7-(8-ethynylnaphthalen-1-yl)-8-fluoropyrido[4,3-d]pyrimidin-2-yl)oxy)methyl)-1-methylpyrrolidin-3-yl)piperazine-1-carbonyl)piperidin-1-yl)-2-(2,6-dioxopiperidin-3-yl)isoindoline-1,3-dione hydrochloride

HNMR (DMSO-d₆, 400 MHz): δ 11.09-11.02 (m, 1H), 9.08-9.02 (m, 1H), 8.17-8.09 (m, 2H), 7.75-7.63 (m, 3H), 7.61-7.53 (m, 2H), 7.34-7.19 (m, 2H), 5.10-5.01 (m, 1H), 4.53-4.39 (m, 2H), 4.38-4.27 (m, 2H), 4.09-3.97 (m, 2H), 3.69-3.39 (m, 12H), 3.10-2.81 (m, 7H), 2.63-2.55 (m, 2H), 2.30 (s, 4H), 2.16-1.95 (m, 4H), 1.74-1.59 (m, 9H). LC-MS: m/z 974.4 [M−HCl+H]⁺.

### Compound 9:

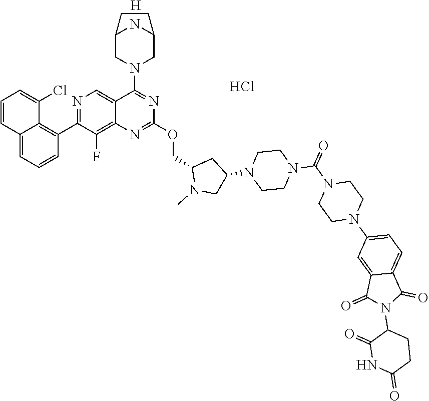

### 5-(4-(1-((3S,5S)-5-(((4-(3,8-diazabicyclo[3.2.1]octan-3-yl)-7-(8-chloronaphthalen-1-yl)-8-fluoropyrido[4,3-d]pyrimidin-2-yl)oxy)methyl)-1-methylpyrrolidin-3-yl)piperidine-4-carbonyl)piperazin-1-yl)-2-(2,6-dioxopiperidin-3-yl)isoindoline-1,3-dione hydrochloride

HNMR (DMSO-d₆, 400 MHz): δ 11.10-11.04 (m, 1H), 9.11-9.07 (m, 1H), 8.16 (s, 1H), 8.11-8.06 (m, 1H), 7.75-7.68 (m, 2H), 7.67-7.59 (m, 2H), 7.58-7.53 (m, 1H), 7.35-7.31 (m, 1H), 7.26-7.20 (m, 1H), 5.12-5.03 (m, 1H), 4.49-4.40 (m, 3H), 4.39-4.32 (m, 1H), 3.66-3.57 (m, 8H), 3.51-3.41 (m, 5H), 3.05-2.96 (m, 2H), 2.93-2.79 (m, 3H), 2.64-2.55 (m, 3H), 2.54-2.53 (m, 1H), 2.31-2.29 (m, 3H), 2.12-1.98 (m, 5H), 1.70-1.54 (m, 9H). LC-MS: m/z 984.5 [M−HCl+H]⁺.

### Compound 10:

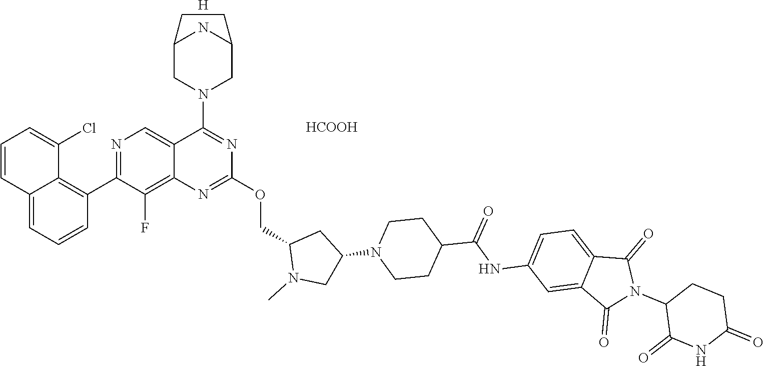

### 1-((3S,5S)-5-(((4-(3,8-diazabicyclo[3.2.1]octan-3-yl)-7-(8-chloronaphthalen-1-yl)-8-fluoropyrido[4,3-d]pyrimidin-2-yl)oxy)methyl)-1-methylpyrrolidin-3-yl)-N-(2-(2,6-dioxopiperidin-3-yl)-1,3-dioxoisoindolin-5-yl)piperidine-4-carboxamide formate

¹H NMR (400 MHz, DMSO-d₆): δ 11.11 (s, 1H), 10.51 (s, 1H), 9.10 (s, 1H), 8.26 (d, J=1.5 Hz, 1H), 8.22-8.18 (m, 2H), 8.10 (d, J=8.3 Hz, 1H), 7.95-7.91 (m, 1H), 7.88-7.84 (m, 1H), 7.75-7.70 (m, 1H), 7.67-7.60 (m, 2H), 7.59-7.54 (m, 1H), 5.12 (dd, J=5.4, 12.8 Hz, 1H), 4.50-4.31 (m, 4H), 3.66-3.56 (m, 6H), 3.04-2.84 (m, 6H), 2.66-2.60 (m, 1H), 2.32 (s, 3H), 2.17-1.94 (m, 6H), 1.85-1.77 (m, 2H), 1.65 (s, 4H). LC-MS: m/z 915.2 [M−HCOOH+H]⁺

### Compound 11:

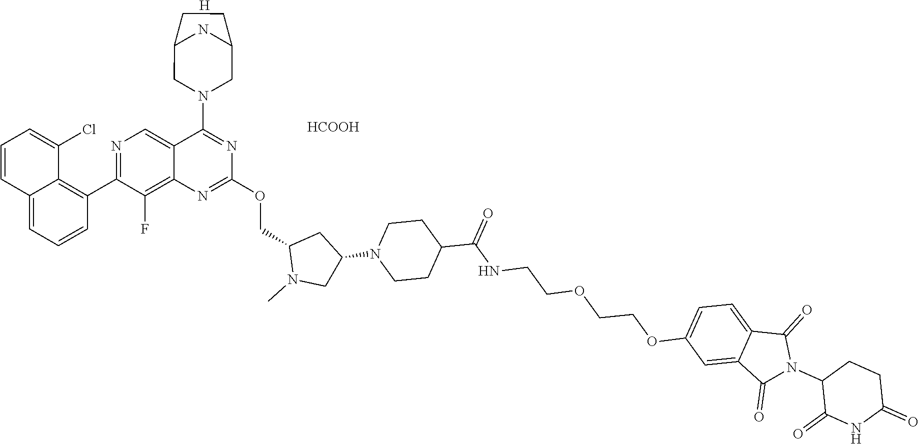

### 1-((3S,5S)-5-(((4-(3,8-diazabicyclo[3.2.1]octan-3-yl)-7-(8-chloronaphthalen-1-yl)-8-fluoropyrido[4,3-d]pyrimidin-2-yl)oxy)methyl)-1-methylpyrrolidin-3-yl)-N-(2-(2-((2-(2,6-dioxopiperidin-3-yl)-1,3-dioxoisoindolin-5-yl)oxy)ethoxy)ethyl)piperidine-4-carboxamide formate

¹H NMR (400 MHz, MeOD): δ 9.11 (s, 1H), 8.47 (s, 1H), 8.16 (d, J=8.3 Hz, 1H), 8.03 (dd, J=1.0, 8.1 Hz, 1H), 7.83-7.76 (m, 1H), 7.74-7.67 (m, 1H), 7.64-7.58 (m, 2H), 7.56-7.50 (m, 1H), 7.41 (t, J=2.0 Hz, 1H), 7.36-7.30 (m, 1H), 5.17-5.07 (m, 1H), 4.80-4.70 (m, 4H), 4.58 (dd, J=4.4, 11.9 Hz, 1H), 4.29-4.22 (m, 2H), 4.02 (br s, 2H), 3.93-3.77 (m, 5H), 3.60-3.52 (m, 3H), 3.43-3.36 (m, 3H), 3.09-2.97 (m, 2H), 2.89-2.65 (m, 6H), 2.60 (d, J=3.1 Hz, 3H), 2.51-2.43 (m, 1H), 2.40-2.33 (m, 1H), 2.19-2.10 (m, 2H), 2.01 (d, J=11.8 Hz, 3H), 1.92-1.78 (m, 4H). LC-MS: m/z 1003.5 [M−HCOOH+H]⁺.

### Compound 12:

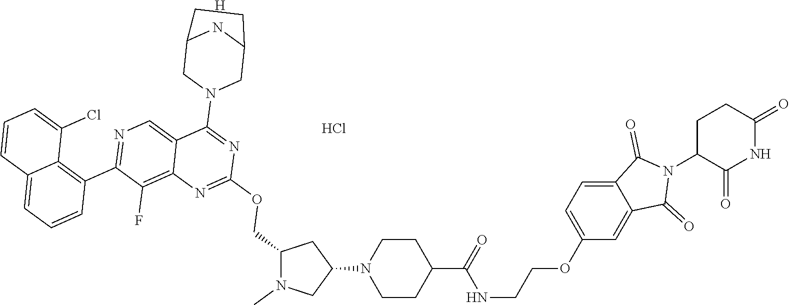

### 1-((3S,5S)-5-(((4-(3,8-diazabicyclo[3.2.1]octan-3-yl)-7-(8-chloronaphthalen-1-yl)-8-fluoropyrido[4,3-d]pyrimidin-2-yl)oxy)methyl)-1-methylpyrrolidin-3-yl)-N-(2-((2-(2,6-dioxopiperidin-3-yl)-1,3-dioxoisoindolin-5-yl)oxy)ethyl)piperidine-4-carboxamide hydrochloride

¹H NMR (DMSO-d₆, 400 MHz): δ 11.11 (s, 1H), 9.09 (s, 1H), 8.19 (d, J=2.6 Hz, 2H), 8.10 (d, J=8.3 Hz, 1H), 7.98 (br t, J=5.4 Hz, 1H), 7.84 (d, J=8.3 Hz, 1H), 7.76-7.69 (m, 1H), 7.67-7.54 (m, 3H), 7.43 (d, J=2.1 Hz, 1H), 7.35 (dd, J=2.3, 8.4 Hz, 1H), 5.12 (dd, J=5.6, 12.9 Hz, 1H), 4.48-4.31 (m, 4H), 4.19 (br t, J=5.7 Hz, 2H), 3.64 (dd, J=3.4, 5.8 Hz, 6H), 3.02-2.94 (m, 2H), 2.93-2.83 (m, 3H), 2.83-2.78 (m, 1H), 2.68 (t, J=1.7 Hz, 1H), 2.35-2.32 (m, 2H), 2.30 (s, 3H), 2.14-2.01 (m, 4H), 1.98-1.88 (m, 2H), 1.70-1.53 (m, 8H). LCMS: m/z 959.4 [M−HCl+H]⁺.

### Compound 13:

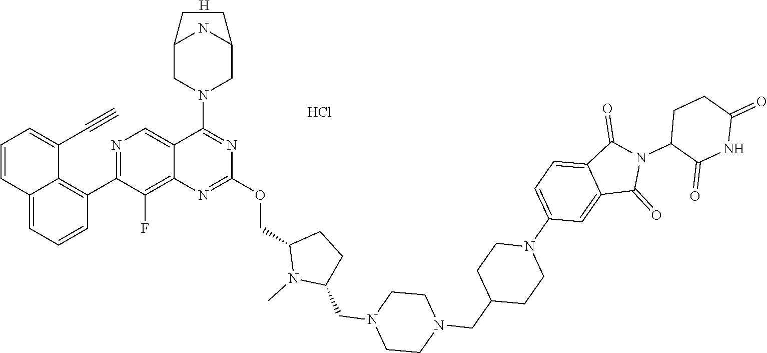

### 5-(4-((4-(((2R,5S)-5-(((4-(3,8-diazabicyclo[3.2.1]octan-3-yl)-7-(8-ethynylnaphthalen-1-yl)-8-fluoropyrido[4,3-d]pyrimidin-2-yl)oxy)methyl)-1-methylpyrrolidin-2-yl)methyl)piperazin-1-yl)methyl)piperidin-1-yl)-2-(2,6-dioxopiperidin-3-yl)isoindoline-1,3-dione hydrochloride

¹H NMR (400 MHz, MeOD): δ 9.07 (s, 1H), 8.16-8.05 (m, 2H), 7.79-7.74 (m, 1H), 7.73-7.65 (m, 2H), 7.61 (dd, J=1.1, 7.0 Hz, 1H), 7.58-7.50 (m, 1H), 7.33 (dd, J=2.2, 7.9 Hz, 1H), 7.21 (ddd, J=2.3, 5.9, 8.6 Hz, 1H), 5.13-5.04 (m, 1H), 4.76-4.57 (m, 5H), 4.07-3.96 (m, 2H), 3.82-3.73 (m, 4H), 3.14 (d, J=8.4 Hz, 1H), 2.99-2.50 (m, 20H), 2.33-2.25 (m, 2H), 2.19-2.09 (m, 3H), 1.97-1.83 (m, 8H), 1.35-1.23 (m, 3H). LC-MS: m/z 974.5 [M−HCl+H]⁺.

### Compound 14:

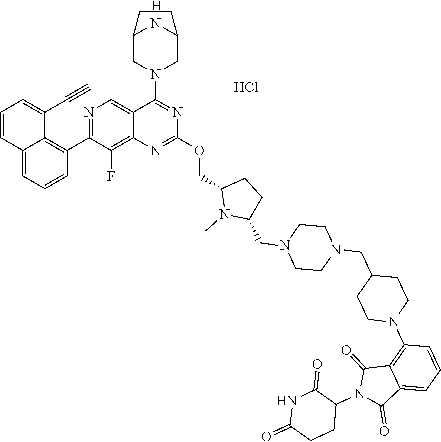

### 4-(4-((4-(((2R,5S)-5-(((4-(3,8-diazabicyclo[3.2.1]octan-3-yl)-7-(8-ethynylnaphthalen-1-yl)-8-fluoropyrido[4,3-d]pyrimidin-2-yl)oxy)methyl)-1-methylpyrrolidin-2-yl)methyl)piperazin-1-yl)methyl)piperidin-1-yl)-2-(2,6-dioxopiperidin-3-yl)isoindoline-1,3-dione

¹HNMR (CDCl₃, 400 MHz): δ 9.04 (s, 1H), 8.39 (s, 1H), 8.09-7.90 (m, 2H), 7.77 (d, J=7.4 Hz, 1H), 7.67-7.52 (m, 3H), 7.51-7.43 (m, 1H), 7.38 (d, J=7.0 Hz, 1H), 7.16 (dd, J=4.5, 8.4 Hz, 1H), 4.97 (dd, J=5.2, 12.2 Hz, 1H), 4.71-4.52 (m, 3H), 4.39 (dt, J=6.7, 10.8 Hz, 1H), 3.88-3.66 (m, 6H), 3.11-2.99 (m, 1H), 2.89-2.75 (m, 9H), 2.71 (dd, J=3.7, 13.1 Hz, 4H), 2.65-2.60 (m, 4H), 2.56-2.50 (m, 1H), 2.41 (d, J=6.4 Hz, 2H), 2.15-2.03 (m, 4H), 1.97-1.69 (m, 10H), 1.55-1.42 (m, 2H). LC-MS: m/z 974.5 [M−HCl+H]⁺.

### Compound 15:

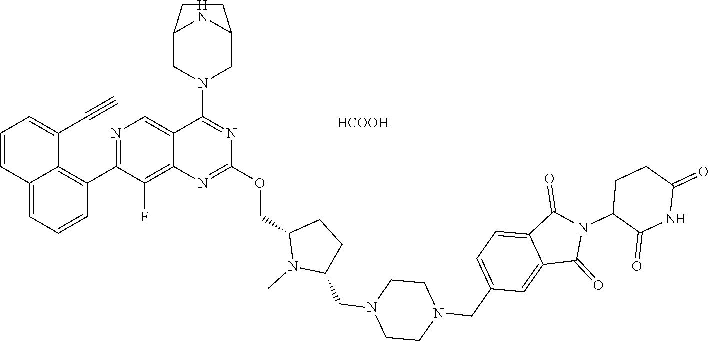 [SMILES: C#Cc1cccc2cccc(-c3ncc4c(N5CC6CCC(C5)N6)nc(OC[C@@H]5CC[C@H](CN6CCN(Cc7ccc8c(c7)C(=O)N(C7CCC(=O)NC7=O)C8=O)CC6)N5C)nc4c3F)c12 ; "HCOOH"]

### 5-((4-(((2R,5S)-5-(((4-(3,8-diazabicyclo[3.2.1]octan-3-yl)-7-(8-ethynylnaphthalen-1-yl)-8-fluoropyrido[4,3-d]pyrimidin-2-yl)oxy)methyl)-1-methylpyrrolidin-2-yl)methyl)piperazin-1-yl)methyl)-2-(2,6-dioxopiperidin-3-yl)isoindoline-1,3-dione formate

¹H NMR (CDCl₃, 400 MHz): δ 9.06 (s, 1H), 8.53 (s, 1H), 8.12-8.02 (m, 2H), 7.77 (d, J=6.6 Hz, 1H), 7.71-7.63 (m, 2H), 7.63-7.59 (m, 1H), 7.53 (J=3.5, 7.7 Hz, 1H), 7.37 (d, J=7.3 Hz, 1H), 7.27 (dd, J=8.5, 15.4 Hz, 1H), 5.16-5.06 (m, 1H), 4.78-4.52 (m, 5H), 3.84-3.67 (m, 6H), 3.13 (d, J=7.9 Hz, 1H), 3.03-2.64 (m, 12H), 2.54 (d, J=4.3 Hz, 3H), 2.42-2.30 (m, 3H), 2.18-2.07 (m, 1H), 2.00-1.73 (m, 8H), 1.54-1.30 (m, 3H). LC-MS: m/z 891.3 [M−HCOOH+H]⁺.

### Compound 16:

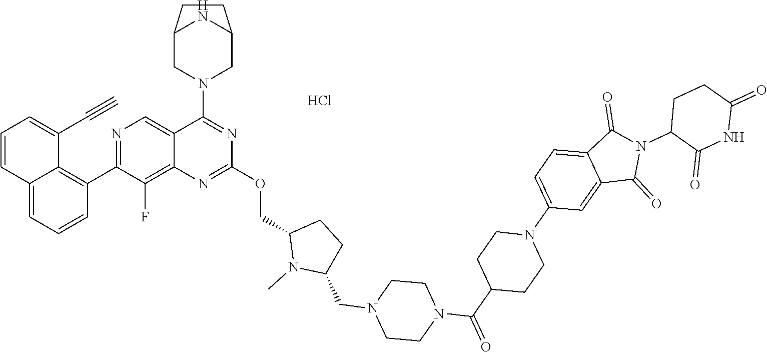 [SMILES: C#Cc1cccc2cccc(-c3ncc4c(N5CC6CCC(C5)N6)nc(OC[C@@H]5CC[C@H](CN6CCN(C(=O)C7CCN(c8ccc9c(c8)C(=O)N(C8CCC(=O)NC8=O)C9=O)CC7)CC6)N5C)nc4c3F)c12 ; Cl]

### 5-(4-(4-(((2R,5S)-5-(((4-(3,8-diazabicyclo[3.2.1]octan-3-yl)-7-(8-ethynylnaphthalen-1-yl)-8-fluoropyrido[4,3-d]pyrimidin-2-yl)oxy)methyl)-1-methylpyrrolidin-2-yl)methyl)piperazine-1-carbonyl)piperidin-1-yl)-2-(2,6-dioxopiperidin-3-yl)isoindoline-1,3-dione hydrochloride

¹H NMR (DMSO-d₆, 400 MHz): δ 11.08 (s, 1H), 9.91-9.86 (m, 1H), 9.57-9.53 (m, 1H), 9.17 (s, 1H), 8.25-8.12 (m, 2H), 7.76-7.65 (m, 3H), 7.62-7.55 (m, 2H), 7.34 (s, 1H), 7.26 (d, J=8.1 Hz, 1H), 5.07 (dd, J=5.3, 12.9 Hz, 1H), 4.89-4.82 (m, 1H), 4.80-4.67 (m, 2H), 4.61-4.53 (m, 1H), 4.22 (s, 2H), 4.08 (d, J=11.3 Hz, 2H), 3.99 (d, J=13.5 Hz, 3H), 3.94-3.81 (m, 2H), 3.71 (d, J=9.8 Hz, 2H), 3.08 (s, 5H), 3.03 (d, J=12.0 Hz, 3H), 2.94-2.83 (m, 2H), 2.70-2.66 (m, 1H), 2.57 (s, 2H), 2.55 (s, 2H), 2.36-2.22 (m, 3H), 2.08-1.87 (m, 8H), 1.78-1.53 (m, 5H). LCMS: m/z 988.6 [M−HCl+H]⁺.

### Compound 17:

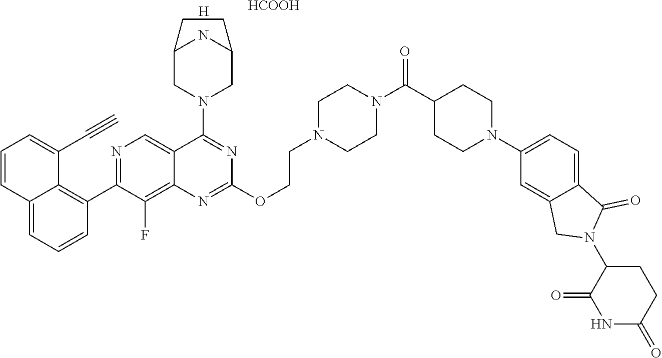

### 3-(5-(4-(4-(2-((4-(3,8-diazabicyclo[3.2.1]octan-3-yl)-7-(8-ethynylnaphthalen-1-yl)-8-fluoropyrido[4,3-d]pyrimidin-2-yl)oxy)ethyl)piperazine-1-carbonyl)piperidin-1-yl)-1-oxoisoindolin-2-yl)piperidine-2,6-dione formate

HNMR (DMSO-d₆, 400 MHz): δ 10.94 (s, 1H), 9.05 (s, 1H), 8.14 (t, J=7.6 Hz, 2H), 7.74-7.67 (m, 2H), 7.61-7.54 (m, 2H), 7.50 (d, J=8.4 Hz, 1H), 7.06-7.02 (m, 2H), 5.04 (dd, J=4.8, 13.3 Hz, 1H), 4.52-4.43 (m, 3H), 4.34 (s, 1H), 4.32-4.30 (m, 1H), 4.22-4.17 (m, 1H), 3.91-3.84 (m, 2H), 3.68-3.62 (m, 2H), 3.59-3.50 (m, 6H), 2.97-2.82 (m, 6H), 2.74 (br t, J=5.6 Hz, 2H), 2.60 (br d, J=1.6 Hz, 2H), 2.38-2.30 (m, 3H), 1.97-1.93 (m, 1H), 1.72-1.59 (m, 9H). LC-MS: m/z 891.5 [M−HCOOH+H]⁺

### Compound 18:

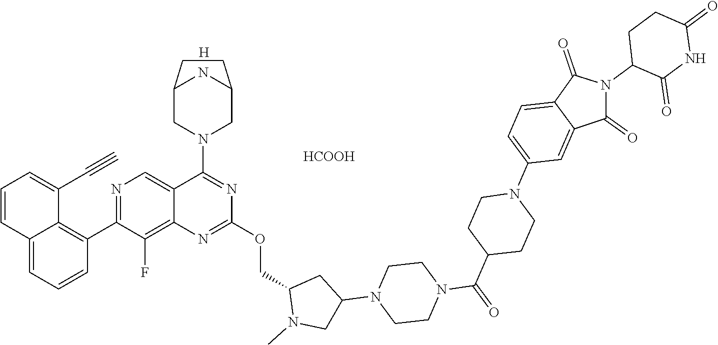

### 3-(5-(4-(4-((3S,5S)-5-(((4-(3,8-diazabicyclo[3.2.1]octan-3-yl)-8-fluoro-7-(8-((triisopropylsilyl)ethynyl)naphthalen-1-yl)pyrido[4,3-d]pyrimidin-2-yl)oxy)methyl)-1-methylpyrrolidin-3-yl)piperazine-1-carbonyl)piperidin-1-yl)-1-oxoisoindolin-2-yl)piperidine-2,6-dione formate

¹H NMR (400 MHz, DMSO-d₆): δ 10.95 (s, 1H), 9.08-9.03 (m, 1H), 8.22 (s, 1H), 8.14 (t, J=7.5 Hz, 2H), 7.76-7.66 (m, 2H), 7.62-7.54 (m, 2H), 7.51 (d, J=8.6 Hz, 1H), 7.09-6.99 (m, 2H), 5.05 (dd, J=4.8, 13.4 Hz, 1H), 4.53-4.41 (m, 2H), 4.37-4.28 (m, 3H), 4.23-4.17 (m, 1H), 3.91-3.85 (m, 2H), 3.67 (s, 2H), 3.63 (s, 1H), 3.58 (s, 4H), 3.52 (s, 3H), 3.02 (dd, J=4.8, 10.3 Hz, 2H), 2.94-2.85 (m, 4H), 2.68 (d, J=1.6 Hz, 2H), 2.61 (s, 3H), 2.42 (d, J=3.0 Hz, 1H), 2.35-2.30 (m, 6H), 2.17-1.91 (m, 3H), 1.67 (s, 6H). LC-MS: m/z 960.1 [M−HCOOH+H]⁺.

### Compound 19:

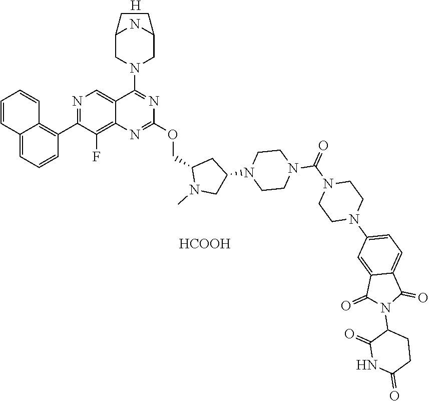

### 5-(4-(1-((3S,5S)-5-(((4-(3,8-diazabicyclo[3.2.1]octan-3-yl)-8-fluoro-7-(naphthalen-1-yl)pyrido[4,3-d]pyrimidin-2-yl)oxy)methyl)-1-methylpyrrolidin-3-yl)piperidine-4-carbonyl)piperazin-1-yl)-2-(2,6-dioxopiperidin-3-yl)isoindoline-1,3-dione formate

¹H NMR (400 MHz, DMSO-d₆+D₂O): δ 9.12 (s, 1H), 8.32 (s, 1H), 8.04-7.94 (m, 2H), 7.69-7.58 (m, 4H), 7.56-7.44 (m, 2H), 7.24-7.12 (m, 2H), 5.00 (dd, J=5.4, 12.7 Hz, 1H), 4.65-4.52 (m, 3H), 4.49-4.42 (m, 1H), 3.94-3.87 (m, 2H), 3.78 (t, J=11.4 Hz, 2H), 3.66-3.52 (m, 4H), 3.46-3.30 (m, 5H), 3.17 (d, J=9.6 Hz, 3H), 2.81 (d, J=4.3 Hz, 2H), 2.69-2.63 (m, 2H), 2.40-2.31 (m, 4H), 2.08-1.98 (m, 1H), 1.91-1.64 (m, 10H). LC-MS: m/z 950.5 [M−HCOOH+H]⁺.

### Compound 20:

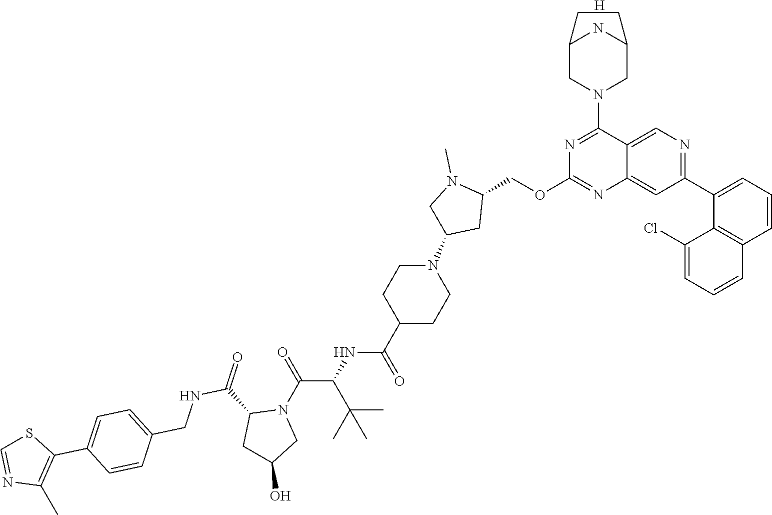

### 1-[(3S,5S)-5-({[7-(8-chloronaphthalen-1-yl)-4-{3,8-diazabicyclo[3.2.1]octan-3-yl}pyrido[4,3-d]pyrimidin-2-yl]oxy}methyl)-1-methylpyrrolidin-3-yl]-N-[(2S)-1-[(2 S,4R)-4-hydroxy-2-({[4-(4-methyl-1,3-thiazol-5-yl)phenyl]methyl}carbamoyl)pyr rolidin-1-yl]-3,3-dimethyl-1-oxobutan-2-yl]piperidine-4-carboxamide

¹H NMR (DMSO-d₆, 400 MHz): δ 9.17 (s, 1H), 8.98 (s, 1H), 8.55 (t, J=6.1 Hz, 1H), 8.13 (dd, J=8.3, 1.5 Hz, 1H), 8.06 (dd, J=8.1, 1.4 Hz, 1H), 7.77-7.50 (m, 5H), 7.47-7.31 (m, 5H), 5.13 (d, J=3.6 Hz, 1H), 4.51 (d, J=9.3 Hz, 1H), 4.45-4.39 (m, 4H), 4.38-4.25 (m, 3H), 4.24-4.20 (m, 1H), 3.71-3.46 (m, 6H), 2.98-2.94 (m, 1H), 2.92-2.76 (m, 3H), 2.67-2.56 (m, 1H), 2.44 (s, 3H), 2.36-2.30 (m, 5H), 2.13-1.95 (m, 2H), 1.94-1.88 (m, 3H), 1.73-1.37 (m, 9H), 0.92 (s, 9H). LC-MS: m/z 1054.2 [M+H]⁺.

### Compound 21:

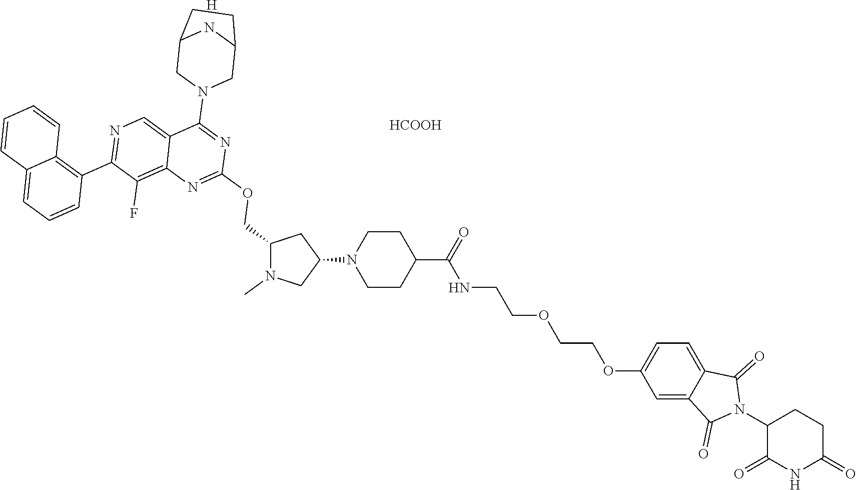

### 1-((3S,5S)-5-(((4-(3,8-diazabicyclo[3.2.1]octan-3-yl)-8-fluoro-7-(naphthalen-1-yl)pyrido[4,3-d]pyrimidin-2-yl)oxy)methyl)-1-methylpyrrolidin-3-yl)-N-(2-(2-((2-(2,6-dioxopiperidin-3-yl)-1,3-dioxoisoindolin-5-yl)oxy)ethoxy)ethyl)piperidine-4-carboxamide formate

¹H NMR (400 MHz, MeOD): δ 9.14 (s, 1H), 8.06 (dd, J=3.6, 5.7 Hz, 1H), 8.01 (d, J=8.5 Hz, 1H), 7.81 (d, J=8.4 Hz, 1H), 7.72 (br d, J=7.8 Hz, 1H), 7.68-7.63 (m, 2H), 7.56 (dt, J=1.2, 7.5 Hz, 1H), 7.52-7.47 (m, 1H), 7.43 (s, 1H), 7.36-7.32 (m, 1H), 5.11 (dd, J=5.5, 12.5 Hz, 1H), 4.70-4.64 (m, 3H), 4.62-4.53 (m, 3H), 4.31-4.27 (m, 2H), 3.87-3.84 (m, 2H), 3.78-3.66 (m, 5H), 3.61 (t, J=5.3 Hz, 2H), 3.38 (br t, J=5.3 Hz, 2H), 3.17-3.13 (m, 1H), 3.05-2.85 (m, 5H), 2.77-2.69 (m, 2H), 2.62-2.50 (m, 2H), 2.48 (s, 3H), 2.31-2.24 (m, 1H), 2.17-2.10 (m, 3H), 1.89-1.81 (m, 4H), 1.77-1.72 (m, 4H). LCMS: m/z 952.4 [M−HCOOH+H]⁺.

### Compound 22:

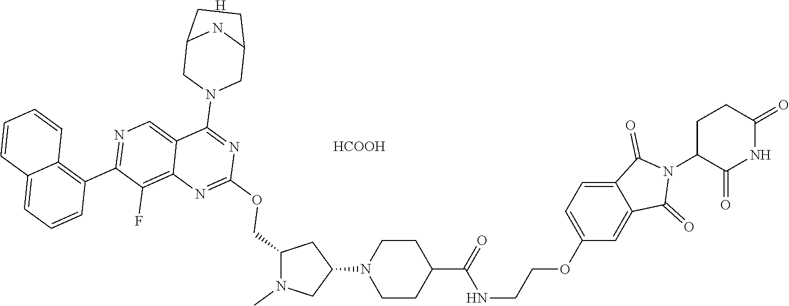

### 1-((3S,5S)-5-(((4-(3,8-diazabicyclo[3.2.1]octan-3-yl)-8-fluoro-7-(naphthalen-1-yl)pyrido[4,3-d]pyrimidin-2-yl)oxy)methyl)-1-methylpyrrolidin-3-yl)-N-(2-((2-(2,6-dioxopiperidin-3-yl)-1,3-dioxoisoindolin-5-yl)oxy)ethyl)piperidine-4-carboxamide formate

¹H NMR (400 MHz, DMSO-d₆+D₂O): δ 9.18 (s, 1H), 8.24 (s, 1H), 8.11-8.02 (m, 2H), 7.83 (d, J=8.3 Hz, 1H), 7.74-7.65 (m, 3H), 7.61-7.48 (m, 2H), 7.41 (d, J=1.8 Hz, 1H), 7.37-7.32 (m, 1H), 5.10 (dd, J=5.4, 12.8 Hz, 1H), 4.60-4.34 (m, 4H), 4.21-4.15 (m, 2H), 3.82-3.64 (m, 4H), 3.45 (d, J=4.0 Hz, 2H), 3.08-2.82 (m, 5H), 2.57 (s, 1H), 2.31 (s, 2H), 2.15-1.99 (m, 5H), 1.80-1.49 (m, 10H). LC-MS: m/z 925.8 [M−HCOOH+H]⁺

### Compound 23:

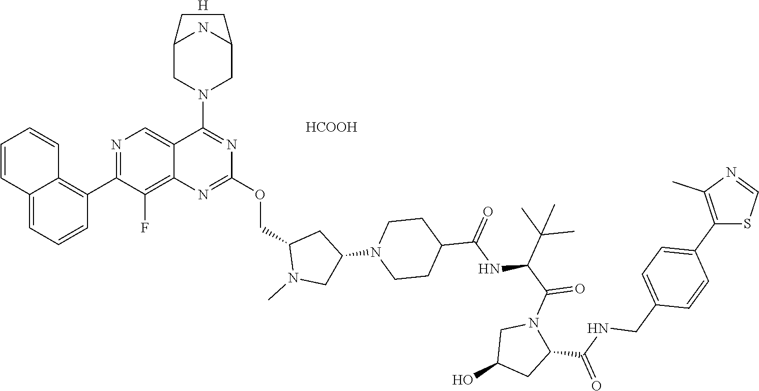

### 1-((3S,5S)-5-(((4-(3,8-diazabicyclo[3.2.1]octan-3-yl)-8-fluoro-7-(naphthalen-1-yl)pyrido[4,3-d]pyrimidin-2-yl)oxy)methyl)-1-methylpyrrolidin-3-yl)-N—((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)piperidine-4-carboxamide formate

¹H NMR (400 MHz, MeOD): δ 9.15 (s, 1H), 8.89 (s, 1H), 8.09-8.05 (m, 1H), 8.01 (d, J=8.3 Hz, 1H), 7.71 (d, J=8.1 Hz, 1H), 7.67-7.63 (m, 2H), 7.59-7.54 (m, 1H), 7.52-7.46 (m, 3H), 7.45-7.45 (m, 1H), 7.45-7.41 (m, 1H), 4.63-4.57 (m, 11H), 4.37 (d, J=15.4 Hz, 1H), 3.93-3.88 (m, 1H), 3.81 (dd, J=3.5, 11.0 Hz, 1H), 3.74 (d, J=12.5 Hz, 2H), 3.68 (s, 2H), 3.19-2.97 (m, 5H), 2.93-2.86 (m, 1H), 2.63-2.56 (m, 1H), 2.50-2.47 (m, 6H), 2.44-2.36 (m, 1H), 2.29-2.05 (m, 5H), 1.89-1.76 (m, 8H), 1.32 (s, 2H), 1.04 (s, 9H). LC-MS: m/z 1038.3 [M−HCOOH+H]⁺.

### Compound 24:

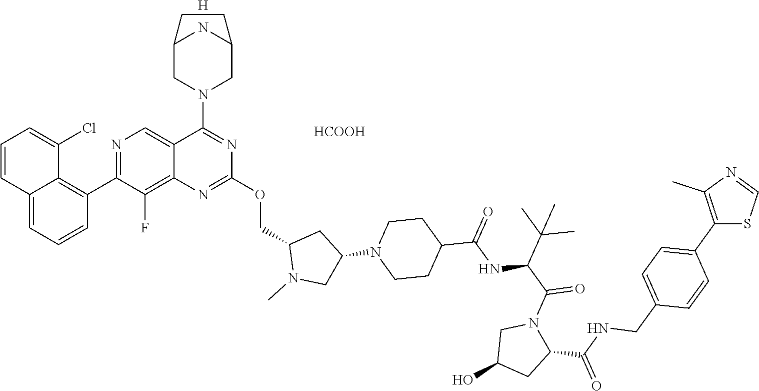

### 1-((3S,5S)-5-(((4-(3,8-diazabicyclo[3.2.1]octan-3-yl)-7-(8-chloronaphthalen-1-yl)-8-fluoropyrido[4,3-d]pyrimidin-2-yl)oxy)methyl)-1-methylpyrrolidin-3-yl)-N—((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)piperidine-4-carboxamide formate

¹HNMR (DMSO-d₆, 400 MHz): δ 9.08 (s, 1H), 8.98 (s, 1H), 8.56 (s, 1H), 8.22-8.17 (m, 1H), 8.10 (d, J=7.8 Hz, 1H), 7.75-7.69 (m, 2H), 7.68-7.53 (m, 3H), 7.44-7.36 (m, 4H), 5.21-5.07 (m, 1H), 4.51 (d, J=9.3 Hz, 1H), 4.42 (t, J=7.6 Hz, 5H), 4.37-4.28 (m, 2H), 4.27-4.17 (m, 1H), 3.68-3.57 (m, 4H), 3.55 (d, J=1.9 Hz, 2H), 3.01-2.95 (m, 1H), 2.91-2.79 (m, 3H), 2.64-2.58 (m, 4H), 2.44 (s, 3H), 2.30 (s, 4H), 2.10-2.00 (m, 2H), 1.95-1.85 (m, 3H), 1.71-1.60 (m, 5H), 1.56-1.50 (m, 2H), 0.93 (s, 9H). LCMS: m/z 1072.1 [M−HCOOH+H]⁺.

### Compound 25:

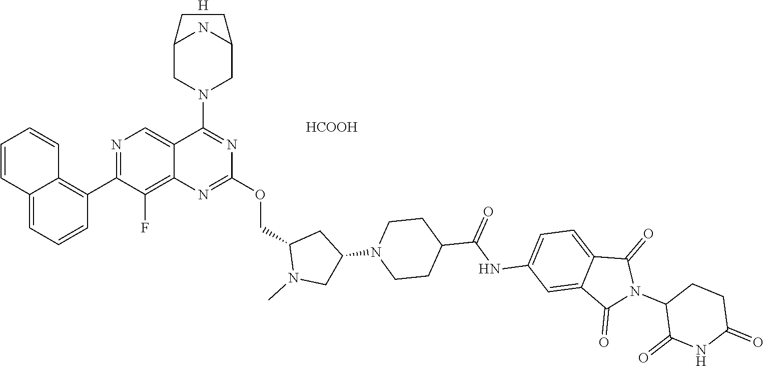

### 1-((3S,5S)-5-(((4-(3,8-diazabicyclo[3.2.1]octan-3-yl)-8-fluoro-7-(naphthalen-1-yl)pyrido[4,3-d]pyrimidin-2-yl)oxy)methyl)-1-methylpyrrolidin-3-yl)-N-(2-(2,6-dioxopiperidin-3-yl)-1,3-dioxoisoindolin-5-yl)piperidine-4-carboxamide formate

¹H NMR (400 MHz, DMSO-d₆) δ 11.11 (s, 1H), 10.51 (s, 1H), 9.19 (s, 1H), 8.28-8.20 (m, 2H), 8.12-8.03 (m, 2H), 7.95-7.91 (m, 1H), 7.89-7.84 (m, 1H), 7.74 (br d, J=8.4 Hz, 1H), 7.69-7.66 (m, 2H), 7.61-7.55 (m, 1H), 7.55-7.49 (m, 1H), 5.12 (dd, J=5.3, 12.8 Hz, 1H), 4.51-4.44 (m, 3H), 4.36 (dd, J=5.6, 10.9 Hz, 1H), 3.66-3.59 (m, 2H), 3.56 (br s, 2H), 3.03-2.84 (m, 6H), 2.65-2.60 (m, 1H), 2.32 (s, 3H), 2.13-1.96 (m, 6H), 1.84-1.78 (m, 2H), 1.65 (s, 5H), 1.24 (s, 1H). LC-MS: m/z 881.6 [M−HCOOH+H]⁺.

### Compound 26:

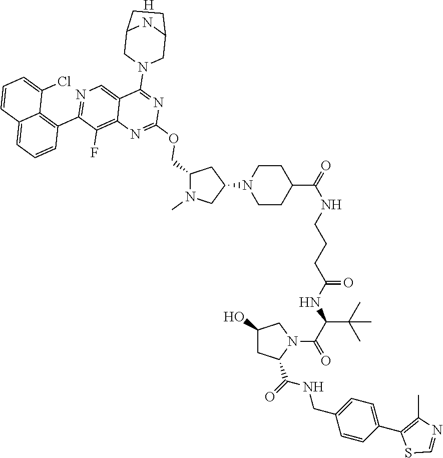

### 1-((3S,5S)-5-(((4-(3,8-diazabicyclo[3.2.1]octan-3-yl)-7-(8-chloronaphthalen-1-yl)-8-fluoropyrido[4,3-d]pyrimidin-2-yl)oxy)methyl)-1-methylpyrrolidin-3-yl)-N-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-4-oxobutyl)piperidine-4-carboxamide

¹H NMR (DMSO-d₆, 400 MHz): δ 9.10-9.06 (m, 1H), 8.98 (s, 1H), 8.60-8.54 (m, 1H), 8.20 (d, J=7.5 Hz, 1H), 8.10 (d, J=7.4 Hz, 1H), 7.91 (d, J=9.3 Hz, 1H), 7.73-7.56 (m, 5H), 7.43-7.37 (m, 4H), 5.23-5.08 (m, 1H), 4.54 (d, J=9.4 Hz, 1H), 4.48-4.40 (m, 5H), 4.37-4.30 (m, 2H), 4.22 (dd, J=5.4, 15.9 Hz, 1H), 3.67-3.58 (m, 4H), 3.54 (br s, 2H), 3.04-2.95 (m, 3H), 2.92-2.84 (m, 2H), 2.83-2.79 (m, 1H), 2.61 (d, J=4.4 Hz, 2H), 2.44 (s, 3H), 2.30 (s, 3H), 2.27-2.21 (m, 1H), 2.13 (d, J=7.5 Hz, 1H), 2.04 (dd, J=5.4, 12.1 Hz, 3H), 1.95-1.85 (m, 3H), 1.66-1.49 (m, 11H), 0.94 (s, 9H). LC-MS: m/z 1157.6 [M+H]⁺.

### Compound 27:

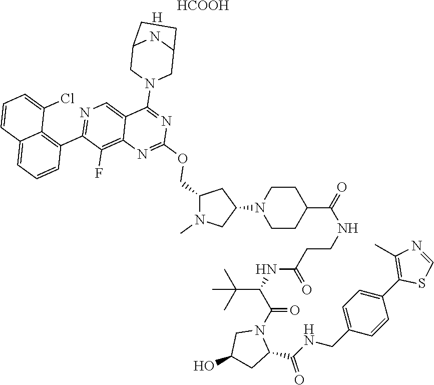

### 1-((3S,5S)-5-(((4-(3,8-diazabicyclo[3.2.1]octan-3-yl)-7-(8-chloronaphthalen-1-yl)-8-fluoropyrido[4,3-d]pyrimidin-2-yl)oxy)methyl)-1-methylpyrrolidin-3-yl)-N-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropyl)piperidine-4-carboxamide formate

HNMR (DMSO-d₆, 400 MHz): δ 9.08 (s, 1H), 8.97 (s, 1H), 8.54 (t, J=6.0 Hz, 1H), 8.26-8.16 (m, 1H), 8.09 (d, J=7.8 Hz, 1H), 7.92 (d, J=9.3 Hz, 1H), 7.72 (t, J=7.6 Hz, 1H), 7.67-7.53 (m, 4H), 7.45-7.35 (m, 4H), 5.13 (s, 1H), 4.52 (d, J=9.5 Hz, 1H), 4.43 (td, J=4.0, 7.7 Hz, 5H), 4.37-4.18 (m, 3H), 3.68-3.50 (m, 6H), 3.23-3.14 (m, 3H), 2.97 (br dd, J=3.8, 9.6 Hz, 1H), 2.94-2.69 (m, 3H), 2.34-2.27 (m, 7H), 2.06-1.96 (m, 3H), 1.95-1.83 (m, 3H), 1.66-1.45 (m, 9H), 1.22-1.12 (m, 3H), 0.97-0.88 (m, 9H). LC-MS: m/z 1143.6 [M−HCOOH+H]⁺.

### Compound 28:

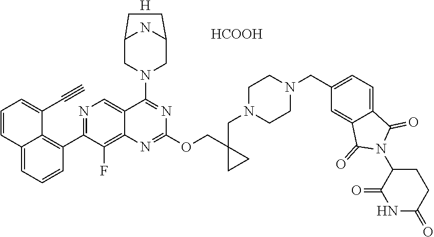

### 5-((4-((1-(((4-(3,8-diazabicyclo[3.2.1]octan-3-yl)-7-(8-ethynylnaphthalen-1-yl)-8-fluoropyrido[4,3-d]pyrimidin-2-yl)oxy)methyl)cyclopropyl)methyl)piperazin-1-yl)methyl)-2-(2,6-dioxopiperidin-3-yl)isoindoline-1,3-dione formate

¹H NMR (400 MHz, DMSO-d₆): δ 11.18-11.06 (m, 1H), 9.04 (s, 1H), 8.25 (s, 1H), 8.15 (t, J=7.8 Hz, 2H), 7.90-7.84 (m, 1H), 7.82-7.76 (m, 2H), 7.74-7.68 (m, 2H), 7.61-7.55 (m, 2H), 5.19-5.11 (m, 1H), 4.52-4.47 (m, 1H), 4.32-4.25 (m, 3H), 3.68-3.65 (m, 2H), 3.63 (s, 2H), 3.56 (s, 4H), 3.53 (s, 2H), 2.95-2.84 (m, 2H), 2.62 (dd, J=3.3, 4.8 Hz, 3H), 2.42-2.38 (m, 3H), 2.32 (d, J=3.3 Hz, 2H), 2.10-2.02 (m, 1H), 1.67 (s, 4H), 0.66-0.60 (m, 2H), 0.46-0.38 (m, 2H). LCMS: m/z 848.4 [M−HCOOH+H]⁺.

### Compound 29:

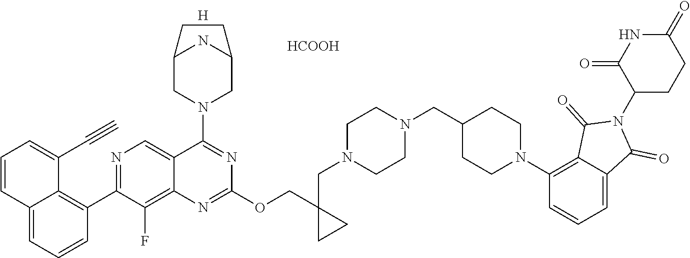

### 4-(4-((4-((1-(((4-(3,8-diazabicyclo[3.2.1]octan-3-yl)-7-(8-ethynylnaphthalen-1-yl)-8-fluoropyrido[4,3-d]pyrimidin-2-yl)oxy)methyl)cyclopropyl)methyl)piperazin-1-yl)methyl)piperidin-1-yl)-2-(2,6-dioxopiperidin-3-yl)isoindoline-1,3-dione formate

¹H NMR (DMSO-d₆ 400 MHz): δ 11.09 (s, 1H), 9.05 (s, 1H), 8.22 (s, 1H), 8.17-8.12 (m, 2H), 7.75-7.65 (m, 3H), 7.60-7.55 (m, 2H), 7.32 (dd, J=4.3, 7.8 Hz, 2H), 5.09 (dd, J=5.4, 12.8 Hz, 1H), 4.51 (d, J=11.8 Hz, 1H), 4.33-4.25 (m, 3H), 3.74-3.62 (m, 5H), 3.62-3.53 (m, 5H), 2.90-2.82 (m, 3H), 2.63-2.56 (m, 3H), 2.32-2.26 (m, 4H), 2.14 (d, J=6.9 Hz, 2H), 2.09-1.95 (m, 2H), 1.78 (d, J=11.0 Hz, 2H), 1.69 (s, 5H), 1.33-1.23 (m, 2H), 0.64 (s, 2H), 0.41 (s, 2H). LC-MS: m/z 931.8 [M−HCOOH+H]⁺.

### Compound 30:

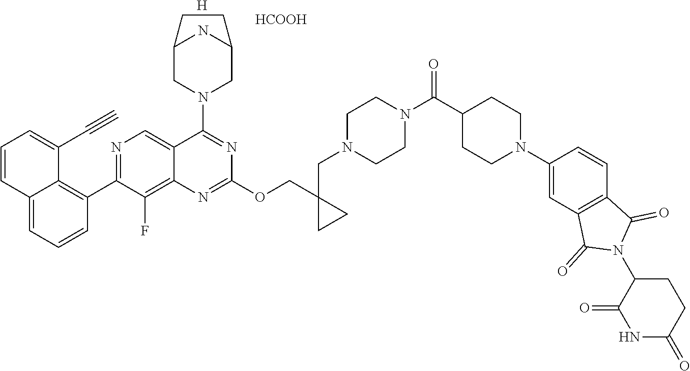

### 5-(4-(4-((1-(((4-(3,8-diazabicyclo[3.2.1]octan-3-yl)-7-(8-ethynylnaphthalen-1-yl)-8-fluoropyrido[4,3-d]pyrimidin-2-yl)oxy)methyl)cyclopropyl)methyl)piperazine-1-carbonyl)piperidin-1-yl)-2-(2,6-dioxopiperidin-3-yl)isoindoline-1,3-dione formate

HNMR (DMSO-d₆, 400 MHz): δ 11.07 (s, 1H), 9.04 (s, 1H), 8.14 (t, J=7.6 Hz, 2H), 7.76-7.63 (m, 3H), 7.60-7.52 (m, 2H), 7.31 (s, 1H), 7.25-7.20 (m, 1H), 5.06 (dd, J=5.3, 12.3 Hz, 1H), 4.56-4.44 (m, 1H), 4.30 (s, 3H), 4.04 (d, J=12.4 Hz, 2H), 3.70-3.60 (m, 3H), 3.54 (d, J=11.5 Hz, 8H), 3.04 (t, J=12.2 Hz, 3H), 2.96-2.82 (m, 3H), 2.34 (s, 5H), 2.04-1.98 (m, 1H), 1.74-1.51 (m, 9H), 0.65 (s, 2H), 0.42 (s, 2H). LC-MS: m/z 945.5 [M−HCOOH+H]⁺.

### Compound 31.

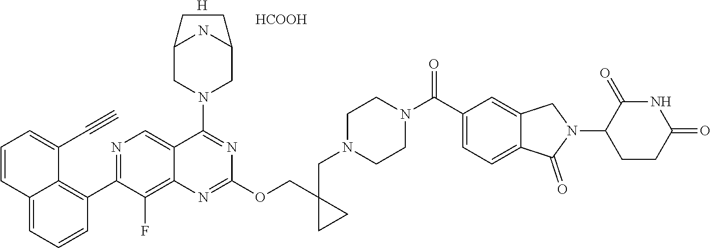

### 3-(5-((4-((1-(((4-(3,8-diazabicyclo[3.2.1]octan-3-yl)-7-(8-ethynylnaphthalen-1-yl)-8-fluoropyrido[4,3-d]pyrimidin-2-yl)oxy)methyl)cyclopropyl)methyl)piperazin-1-yl)methyl)-1-oxoisoindolin-2-yl)piperidine-2,6-dione formate

HNMR (DMSO-d₆, 400 MHz): δ 11.00 (s, 1H), 9.05 (s, 1H), 8.25 (s, 1H), 8.15 (t, J=8.0 Hz, 2H), 7.74-7.65 (m, 3H), 7.61-7.55 (m, 2H), 7.50 (s, 1H), 7.41 (d, J=7.8 Hz, 1H), 5.11 (dd, J=5.1, 13.3 Hz, 1H), 4.57-4.38 (m, 3H), 4.35-4.30 (m, 2H), 4.26 (s, 3H), 3.72-3.63 (m, 6H), 3.60 (d, J=12.5 Hz, 2H), 3.55 (s, 2H), 2.97-2.87 (m, 1H), 2.41-2.34 (m, 5H), 2.31 (s, 2H), 2.02-1.97 (m, 1H), 1.72 (s, 4H), 0.63 (s, 2H), 0.41 (s, 2H). LCMS: m/z 834.5 [M−HCOOH+H]⁺.

The following compounds in Table 1 were prepared according to the methods described above using the appropriate starting materials.

| Com- | | | |
|---|---|---|---|
| pound | | LCMS | |
| Num- | | m/z | ¹H NMR |
| ber | Structure | [M + H]⁺ | (DMSO-d₆, 400 MHz) |
| 32 | 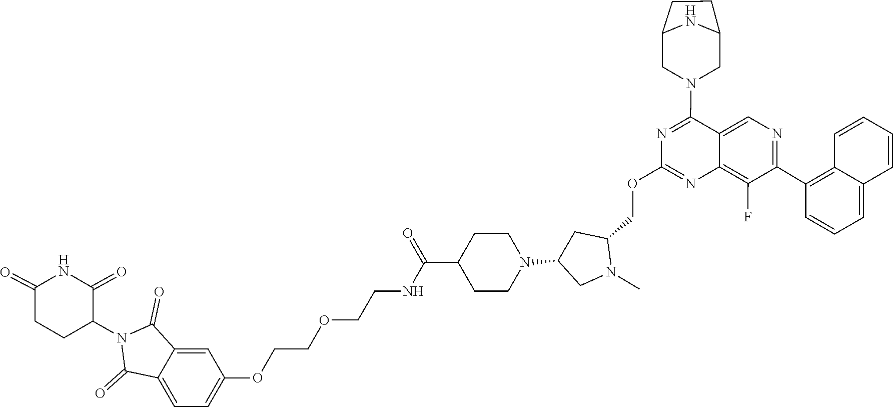 [SMILES: CN1C[C@H](N2CCC(C(=O)NCCOCCOc3ccc4c(c3)C(=O)N(C3CCC(=O)NC3=O)C4=O)CC2)C[C@@H]1COc1nc(N2CC3CCC(C2)N3)c2cnc(-c3cccc4ccccc34)c(F)c2n1] | 952.4 | 9.14 (s, 1H), 8.06 (dd, J = 3.6 Hz, 1H), 8.01 (d, J = 8.4 Hz, 1H), 7.81 (d, J = 8.4 Hz, 1H), 7.72 (d, J = 7.8 Hz, 1H), 7.68-7.63 (m, 2H), 7.56 (dt, J = 1.2 Hz, 1H), 7.52-7.47 (m, 1H), 7.43 (s, 1H), 7.36-7.32 (m, 1H), 5.11 (dd, J = 5.6 Hz, 1H), 4.70-4.64 (m, 3H), 4.62-4.53 (m, 3H), 4.31-4.27 (m, 2H), 3.87-3.84 (m, 2H), 3.78-3.66 (m, 5H), 3.61 (t, J = 5.4 Hz, 2H), 3.38 (t, J = 5.4 Hz, 2H), 3.17- 3.13 (m, 1H), 3.05-2.85 (m, 5H), 2.77- 2.69 (m, 2H), 2.62-2.50 (m, 2H), 2.48 (s, 3H), 2.31-2.24 (m, 1H), 2.17-2.10 (m, 3H), 1.89-1.81 (m, 4H), 1.77-1.72 (m, 4H). |
| 33 | 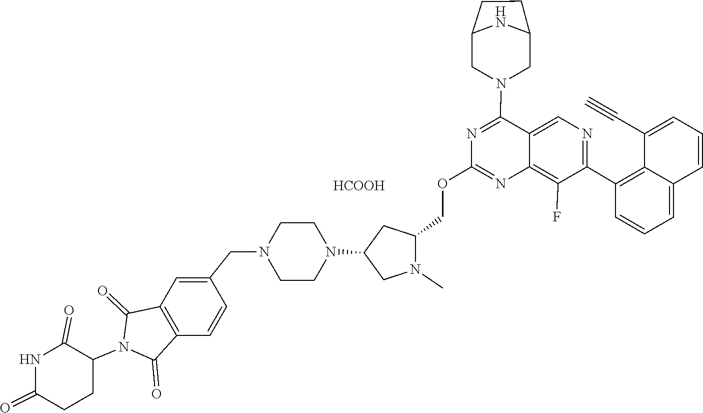 [SMILES: C#Cc1cccc2cccc(-c3ncc4c(N5CC6CCC(C5)N6)nc(OC[C@H]5C[C@@H](N6CCN(Cc7ccc8c(c7)C(=O)N(C7CCC(=O)NC7=O)C8=O)CC6)CN5C)nc4c3F)c12 ; "HCOOH"] | 863.5 | 11.11-10.89 (m, 1H), 9.06 (s, 1H), 8.23 (s, 2H), 8.15 (t, J = 8.0 Hz, 2H), 7.75- 7.71 (m, 1H), 7.67 (d, J = 7.0 Hz, 1H), 7.62-7.55 (m, 2H), 7.52 (s, 1H), 7.43 (d, J = 7.8 Hz, 1H), 5.11 (dd, J = 5.2, 13.3 Hz, 1H), 4.52 (d, J = 13.0 Hz, 1H), 4.47-4.41 (m, 2H), 4.39-4.27 (m, 3H), 3.70 (s, 4H), 3.68-3.65 (m, 2H), 3.64- 3.61 (m, 1H), 3.56 (s, 3H), 3.05-2.98 (m, 1H), 2.97-2.85 (m, 2H), 2.68-2.59 (m, 2H), 2.38 (dd, J = 4.6, 13.1 Hz, 6H), 2.36-2.32 (m, 2H), 2.31 (s, 2H), 2.14-2.06 (m, 1H), 2.04-1.94 (m, 1H), 1.73 (s, 3H), 1.64-1.56 (m, 1H). |
| 34 | 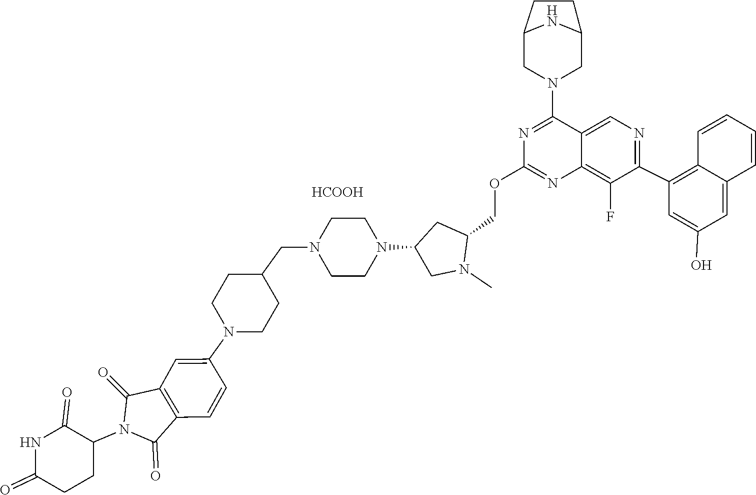 [SMILES: CN1C[C@H](N2CCN(CC3CCN(c4ccc5c(c4)C(=O)N(C4CCC(=O)NC4=O)C5=O)CC3)CC2)C[C@@H]1COc1nc(N2CC3CCC(C2)N3)c2cnc(-c3cc(O)cc4ccccc34)c(F)c2n1 ; "HCOOH"] | 952.3 | 11.15-11.01 (m, 1H), 9.18 (s, 1H), 8.21 (s, 1H), 7.80 (d, J = 8.0 Hz, 1H), 7.65 (d, J = 8.6 Hz, 1H), 7.59-7.53 (m, 1H), 7.47-7.40 (m, 1H), 7.33-7.26 (m, 3H), 7.25-7.19 (m, 3H), 5.10-5.02 (m, 1H), 4.52-4.33 (m, 5H), 4.02 (d, J = 11.8 Hz, 3H), 3.62 (s, 4H), 3.03-2.85 (m, 7H), 2.69-2.59 (m, 3H), 2.35 (s, 2H), 2.31 (s, 5H), 2.25 (s, 1H), 2.11 (d, J = 6.4 Hz, 3H), 2.05-1.96 (m, 2H), 1.76 (d, J = 11.0 Hz, 3H), 1.67 (s, 3H), 1.20-0.96 (m, 3H). |
| 35 | 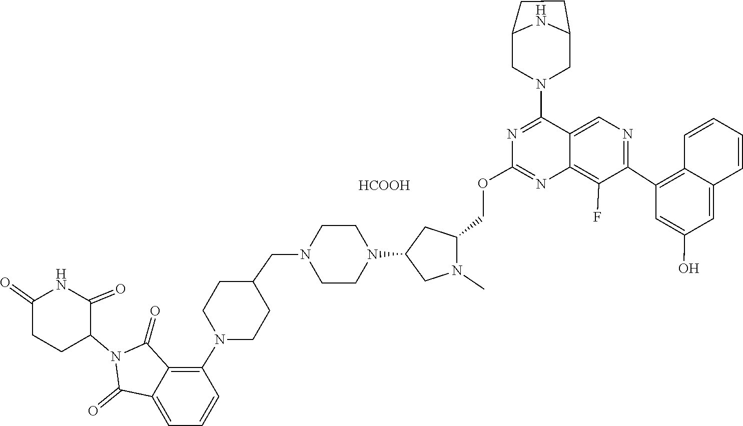 [SMILES: CN1C[C@H](N2CCN(CC3CCN(c4cccc5c4C(=O)N(C4CCC(=O)NC4=O)C5=O)CC3)CC2)C[C@@H]1COc1nc(N2CC3CCC(C2)N3)c2cnc(-c3cc(O)cc4ccccc34)c(F)c2n1 ; "HCOOH"] | 952.8 | 11.12-11.05 (m, 1H), 9.18 (s, 1H), 8.20 (s, 2H), 7.80 (d, J = 8.1 Hz, 1H), 7.70- 7.64 (m, 1H), 7.56 (d, J = 8.8 Hz, 1H), 7.44 (t, J = 7.6 Hz 1H), 7.34-7.22 (m, 5H), 5.08 (dd, J = 5.6, 12.9 Hz, 1H), 4.52-4.42 (m, H), 4.41-4.34 (m, 1H), 3.70-3.57 (m, 11H), 3.05-2.98 (m, 2H), 2.93-2.81 (m, 5H), 2.66-2.59 (m, 3H), 2.42-2.38 (m, 3H), 2.32 (s, 3H), 2.16 (d, J = 6.6 Hz, 2H), 2.09-2.01 (m, 2H), 1.97-1.86 (m, 1H), 1.82-1.76 (m, 2H), 1.67 (s, 3H), 1.64-1.54 (m, 2H), 1.39- 1.21 (m, 3H). |
| 36 | 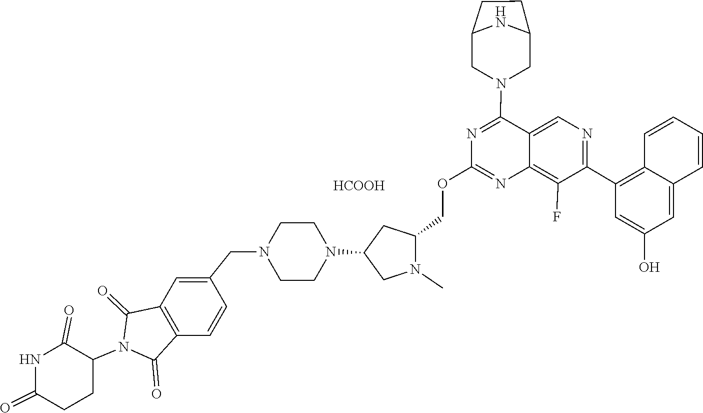 [SMILES: CN1C[C@H](N2CCN(Cc3ccc4c(c3)C(=O)N(C3CCC(=O)NC3=O)C4=O)CC2)C[C@@H]1COc1nc(N2CC3CCC(C2)N3)c2cnc(-c3cc(O)cc4ccccc34)c(F)c2n1 ; "HCOOH"] | 869.3 | 11.20-11.04 (m, 1H), 9.17 (s, 1H), 8.20 (s, 2H), 7.90-7.85 (m, 1H), 7.81 (dd, J = 5.8, 9.0 Hz, 3H), 7.56 (d, J = 8.0 Hz, 1H), 7.44 (t, J = 7.6 Hz, 1H), 7.30-7.21 (m, 3H), 5.18-5.10 (m, 1H), 4.51-4.33 (m, 5H), 3.64 (s, 3H), 3.05-2.98 (m, 3H), 2.90 (ddd, J = 3.8, 11.9, 17.3 Hz, 4H), 2.62 (d, J = 2.4 Hz, 3H), 2.59-2.56 (m, 3H), 2.35-2.32 (m, 3H), 2.31 (s, 3H), 2.12-2.02 (m, 3H), 1.70-1.60 (m, 5H). |
| 37 | 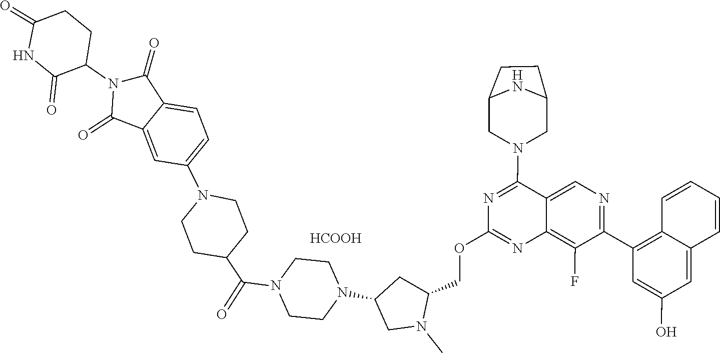 [SMILES: CN1C[C@H](N2CCN(C(=O)C3CCN(c4ccc5c(c4)C(=O)N(C4CCC(=O)NC4=O)C5=O)CC3)CC2)C[C@@H]1COc1nc(N2CC3CCC(C2)N3)c2cnc(-c3cc(O)cc4ccccc34)c(F)c2n1 ; "HCOOH"] | 966.9 | 11.07 (s, 1H), 9.18 (s, 1H), 8.16 (s, 1H), 7.79 (d, J = 8.4 Hz, 1H), 7.65 (d, J = 8.6 Hz, 1H), 7.55 (d, J = 7.8 Hz, 1H), 7.44 (t, J = 7.4 Hz, 1H), 7.34-7.20 (m, 5H), 5.12-5.02 (m, 1H), 4.53-4.43 (m, 3H), 4.38 (dd, J = 5.0, 10.8 Hz, 1H), 4.06-3.99 (m, 2H), 3.64 (s, 7H), 3.09- 2.98 (m, 7H), 2.69-2.58 (m, 4H), 2.42 (d, J = 3.4 Hz, 2H), 2.37-2.28 (m, 8H), 2.19-2.05 (m, 2H), 2.03-1.98 (m, 1H), 1.74-1.63 (m, 7H). |
| 38 | 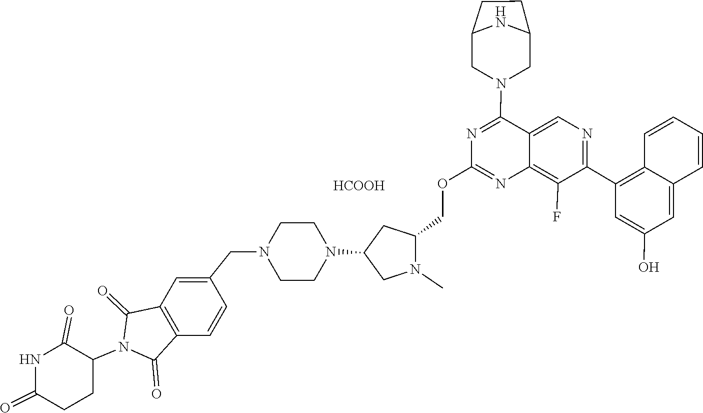 [SMILES: CN1C[C@H](N2CCN(Cc3ccc4c(c3)C(=O)N(C3CCC(=O)NC3=O)C4=O)CC2)C[C@@H]1COc1nc(N2CC3CCC(C2)N3)c2cnc(-c3cc(O)cc4ccccc34)c(F)c2n1 ; "HCOOH"] | 855.3 | 10.99 (s, 1H), 9.18 (s, 1H), 8.22 (s, 2H), 7.80 (d, J = 8.3 Hz, 1H), 7.67 (d, J = 7.8 Hz, 1H), 7.58-7.50 (m, 2H), 7.48- 7.38 (m, 2H), 7.31-7.20 (m, 3H), 5.11 (dd, J = 5.0, 13.3 Hz, 1H), 4.51 (s, 1H), 4.49-4.27 (m, 6H), 3.56 (s, 6H), 3.02 (dd, J = 3.4, 10.1 Hz, 1H), 2.97-2.85 (m, 2H), 2.70-2.61 (m, 2H), 2.60-2.56 (m, 1H), 2.53 (d, J = 1.8 Hz, 2H), 2.44- 2.37 (m, 6H), 2.35 (s, 1H), 2.31 (s, 2H), 2.12-2.06 (m, 1H), 2.02-1.95 (m, 1H), 1.69 (m, 4H), 1.62 (s, 1H). |
| 39 | 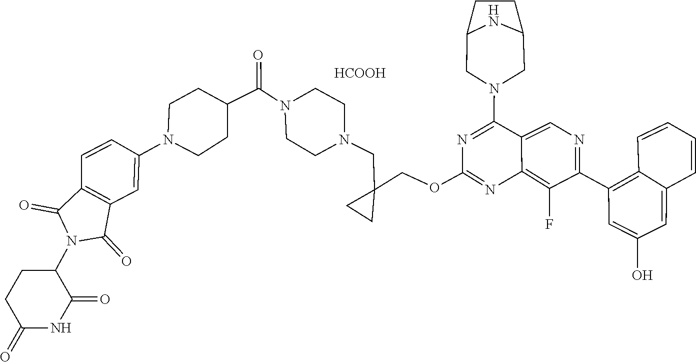 [SMILES: O=C1CCC(N2C(=O)c3ccc(N4CCC(C(=O)N5CCN(CC6(COc7nc(N8CC9CCC(C8)N9)c8cnc(-c9cc(O)cc%10ccccc9%10)c(F)c8n7)CC6)CC5)CC4)cc3C2=O)C(=O)N1 ; "HCOOH"] | 937.2 | 11.07 (s, 1H), 9.17 (s, 1H), 8.17 (s, 1H), 7.80 (d, J = 8.2 Hz, 1H), 7.68-7.63 (m, 1H), 7.54 (d, J = 8.6 Hz, 1H), 7.44 (t, J = 7.4 Hz, 1H), 7.30 (dd, J = 2.0, 8.8 Hz, 2H), 7.26-7.20 (m, 3H), 5.06 (dd, J = 5.4, 12.8 Hz, 1H), 4.50 (d, J = 11.8 Hz, 2H), 4.34 (s, 2H), 4.04 (d, J = 12.6 Hz, 2H), 3.74-3.63 (m, 5H), 3.54-3.46 (m, 4H), 3.04 (t, J = 11.4 Hz, 3H), 2.98- 2.88 (m, 2H), 2.57-2.53 (m, 2H), 2.43 (d, J = 5.4 Hz, 2H), 2.37-2.32 (m, 4H), 2.04-1.98 (m, 1H), 1.74-1.54 (m, 8H), 0.67 (s, 2H), 0.43 (s, 2H). |
| 40 | 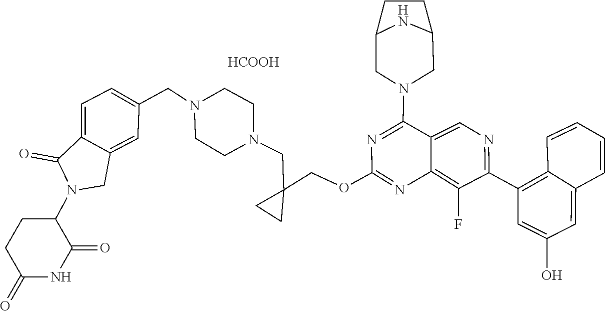 [SMILES: O=C1CCC(N2Cc3cc(CN4CCN(CC5(COc6nc(N7CC8CCC(C7)N8)c7cnc(-c8cc(O)cc9ccccc89)c(F)c7n6)CC5)CC4)ccc3C2=O)C(=O)N1 ; "HCOOH"] | 826.3 | 11.04 (s, 1H), 9.22 (s, 1H), 8.29 (s, 1H), 7.86 (d, J = 8.4 Hz, 1H), 7.72 (d, J = 7.8 Hz, 1H), 7.62 (d, J = 8.4 Hz, 1H), 7.54 (s, 1H), 7.52-7.43 (m, 2H), 7.35 (d, J = 2.3 Hz, 1H), 7.32-7.26 (m, 2H), 5.16 (dd, J = 5.0, 13.3 Hz, 1H), 4.57- 4.45 (m, 3H), 4.39-4.32 (m, 3H), 3.73- 3.71 (m, 3H), 3.68 (s, 5H), 3.02-2.94 (m, 1H), 2.69-2.63 (m, 1H), 2.60-2.58 (m, 2H), 2.45 (d, J = 4.4 Hz, 2H), 2.41 (d, J = 4.4 Hz, 2H), 2.40-2.33 (m, 4H), 2.08-2.02 (m, 1H), 1.74 (s, 4H), 0.69 (s, 2H), 0.46 (s, 2H). |
| 41 | 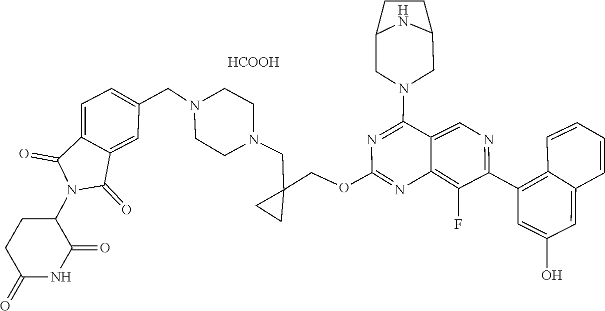 [SMILES: O=C1CCC(N2C(=O)c3ccc(CN4CCN(CC5(COc6nc(N7CC8CCC(C7)N8)c7cnc(-c8cc(O)cc9ccccc89)c(F)c7n6)CC5)CC4)cc3C2=O)C(=O)N1 ; "HCOOH"] | 840.2 | 11.23-11.04 (m, 1H), 9.17 (s, 1H), 8.32- 8.11 (m, 2H), 7.90-7.85 (m, 1H), 7.83- 7.72 (m, 3H), 7.60-7.52 (m, 1H), 7.47- 7.40 (m, 1H), 7.29 (s, 1H), 7.27-7.19 (m, 2H), 5.21-5.05 (m, 1H), 4.48 (d, J = 11.0 Hz, 2H), 4.34-4.26 (m, 2H), 3.61 (s, 4H), 2.95-2.84 (m, 2H), 2.68 (s, 1H), 2.62 (d, J = 1.0 Hz, 1H), 2.57 (d, J = 4.0 Hz, 1H), 2.55 (s, 2H), 2.42-2.36 (m, 4H), 2.32 (s, 4H), 2.09-2.02 (m, 1H), 1.68 (s, 4H), 0.64 (s, 2H), 0.46-0.37 (m, 2H). |
| 42 | 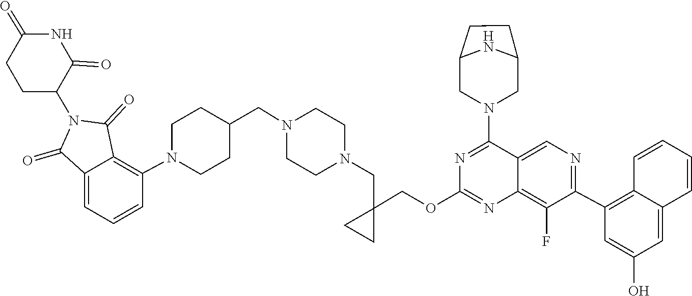 [SMILES: O=C1CCC(N2C(=O)c3cccc(N4CCC(CN5CCN(CC6(COc7nc(N8CC9CCC(C8)N9)c8cnc(-c9cc(O)cc%10ccccc9%10)c(F)c8n7)CC6)CC5)CC4)c3C2=O)C(=O)N1] | 923.5 | — |
| 43 | 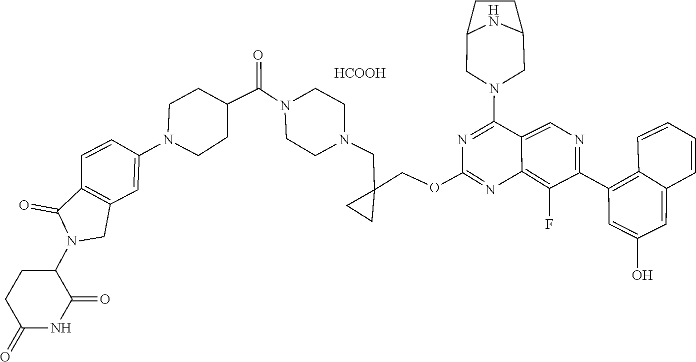 [SMILES: O=C1CCC(N2Cc3cc(N4CCC(C(=O)N5CCN(CC6(COc7nc(N8CC9CCC(C8)N9)c8cnc(-c9cc(O)cc%10ccccc9%10)c(F)c8n7)CC6)CC5)CC4)ccc3C2=O)C(=O)N1 ; "HCOOH"] | 925.3 | 10.94 (s, 1H), 9.17 (s, 1H), 8.19 (s, 1H), 7.81 (d, J = 8.4 Hz, 1H), 7.57-7.42 (m, 3H), 7.30-7.22 (m, 3H), 7.07-7.02 (m, 2H), 5.04 (dd, J = 5.0, 13.3 Hz, 1H), 4.48 (d, J = 11.4 Hz, 2H), 4.36-4.30 (m, 3H), 4.23-4.17 (m, 1H), 3.88 (d, J = 12.6 Hz, 2H), 3.67-3.59 (m, 5H), 3.50 (d, J = 2.0 Hz, 4H), 2.96-2.82 (m, 5H), 2.61 (d, J = 3.0 Hz, 1H), 2.37-2.32 (m, 5H), 1.99-1.93 (m, 1H), 1.68 (s, 4H), 1.66-1.59 (m, 4H), 0.67 (s, 2H), 0.44 (s, 2H). |
| 44 | 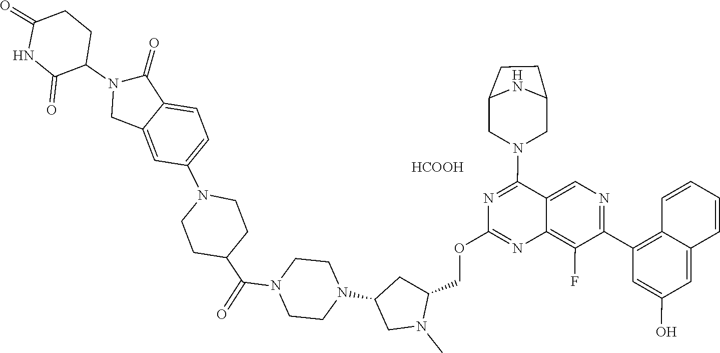 [SMILES: CN1C[C@H](N2CCN(C(=O)C3CCN(c4ccc5c(c4)CN(C4CCC(=O)NC4=O)C5=O)CC3)CC2)C[C@@H]1COc1nc(N2CC3CCC(C2)N3)c2cnc(-c3cc(O)cc4ccccc34)c(F)c2n1 ; "HCOOH"] | 952.4 | 10.94 (s, 1H), 9.18 (s, 1H), 8.18 (s, 1H), 7.80 (d, J = 8.2 Hz, 1H), 7.58-7.42 (m, 3H), 7.30-7.22 (m, 3H), 7.06-7.01 (m, 2H), 5.05 (dd, J = 5.0, 13.3 Hz, 1H), 4.54-4.44 (m, 3H), 4.42-4.17 (m, 4H), 3.93-3.81 (m, 3H), 3.65 (s, 5H), 3.51 (s, 4H), 3.06-2.99 (m, 2H), 2.93-2.85 (m, 4H), 2.69-2.56 (m, 2H), 2.37-2.29 (m, 8H), 2.14-2.08 (m, 1H), 1.98-1.93 (m, 1H), 1.69 (s, 3H), 1.66 (s, 4H). |
| 45 | 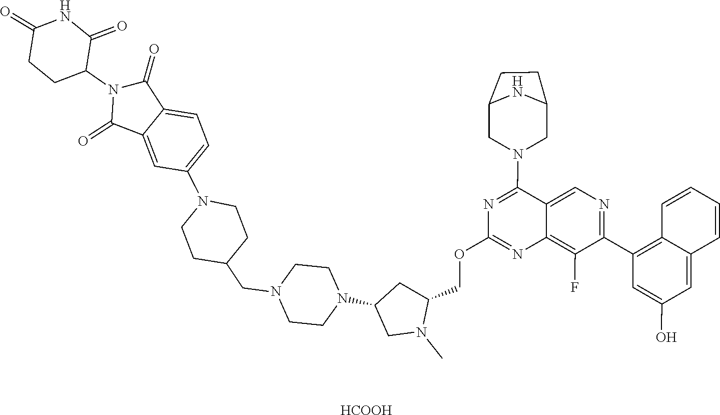 [SMILES: CN1C[C@H](N2CCN(CC3CCN(c4ccc5c(c4)C(=O)N(C4CCC(=O)NC4=O)C5=O)CC3)CC2)C[C@@H]1COc1nc(N2CC3CCC(C2)N3)c2cnc(-c3cc(O)cc4ccccc34)c(F)c2n1 ; "HCOOH"] | 938.3 | 10.95 (s, 1H), 9.18 (s, 1H), 8.21 (s, 2H), 7.80 (d, J = 8.4 Hz, 1H), 7.56 (d, J = 8.4 Hz, 1H), 7.49 (d, J = 9.0 Hz, 1H), 7.44 (t, J = 7.2 Hz, 1H), 7.29 (d, J = 2.0 Hz, 1H), 7.27-7.22 (m, 2H), 7.05-7.00 (m, 2H), 5.04 (dd, J = 5.0, 13.3 Hz, 1H), 4.51-4.43 (m, 3H), 4.39-4.29 (m, 2H), 4.22-4.16 (m, 1H), 3.85 (d, J = 12.0 Hz, 3H), 3.66 (s, 2H), 3.63 (s, 4H), 3.01 (dd, J = 3.2, 10.1 Hz, 1H), 2.95- 2.85 (m, 3H), 2.83-2.76 (m, 2H), 2.69- 2.60 (m, 3H), 2.53 (s, 2H), 2.52 (s, 2H), 2.36-2.33 (m, 3H), 2.31 (s, 3H), 2.11 (d, J = 6.6 Hz, 2H), 1.99-1.92 (m, 1H), 1.79-1.72 (m, 3H), 1.68 (s, 3H), 1.63- 1.56 (m, 1H), 1.20-1.10 (m, 2H). |
| 46 | 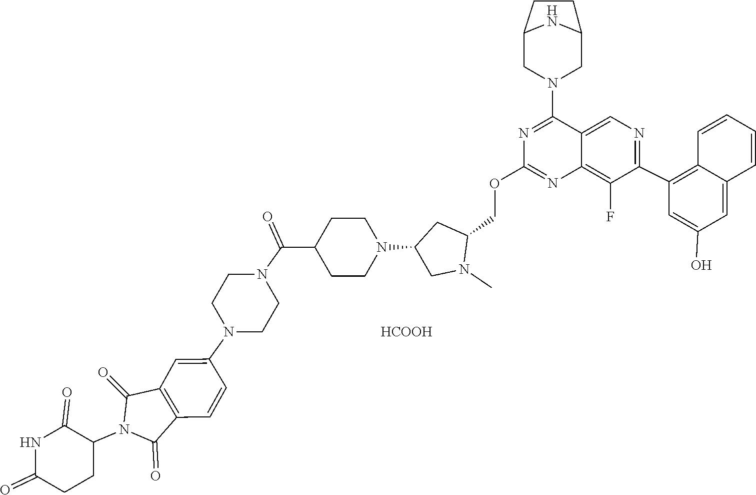 [SMILES: CN1C[C@H](N2CCC(C(=O)N3CCN(c4ccc5c(c4)C(=O)N(C4CCC(=O)NC4=O)C5=O)CC3)CC2)C[C@@H]1COc1nc(N2CC3CCC(C2)N3)c2cnc(-c3cc(O)cc4ccccc34)c(F)c2n1 ; "HCOOH"] | 966.3 | 11.19-11.00 (m, 1H), 9.18 (s, 1H), 8.26 (s, 3H), 7.80 (d, J = 8.4 Hz, 1H), 7.71 (d, J = 8.4 Hz, 1H), 7.56 (br d, J = 8.2 Hz, 1H), 7.44 (t, J = 7.2 Hz, 1H), 7.35 (d, J = 1.9 Hz, 1H), 7.29 (d, J = 2.0 Hz, 1H), 7.25-7.22 (m, 2H), 5.08 (dd, J = 5.3, 12.9 Hz, 1H), 4.47 (dd, J = 4.8, 10.5 Hz, 3H), 4.37 (dd, J = 5.8, 11.0 Hz, 1H), 3.67-3.58 (m, 9H), 3.06-2.81 (m, 6H), 2.65-2.55 (m, 3H), 2.32 (s, 3H), 2.14-1.98 (m, 5H), 1.70-1.51 (m, 10H). |
| 47 | 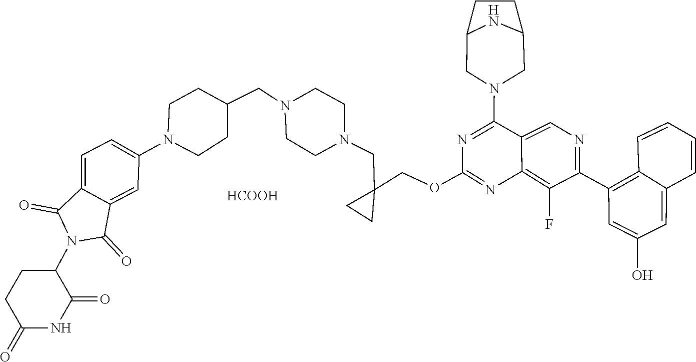 [SMILES: O=C1CCC(N2C(=O)c3ccc(N4CCC(CN5CCN(CC6(COc7nc(N8CC9CCC(C8)N9)c8cnc(-c9cc(O)cc%10ccccc9%10)c(F)c8n7)CC6)CC5)CC4)cc3C2=O)C(=O)N1 ; "HCOOH"] | 923.3 | 11.19 (s, 1H), 9.17 (s, 1H), 8.20 (s, 1H), 7.83-7.78 (m, 1H), 7.67-7.40 (m, 3H), 7.33-7.12 (m, 4H), 5.12-4.93 (m, 1H), 4.54-4.39 (m, 2H), 4.31 (s, 1H), 4.01 (d, J = 12.4 Hz, 2H), 3.69-3.59 (m, 6H), 3.34-3.24 (m, 3H), 3.00-2.84 (m, 3H), 2.60 (d, J = 2.8 Hz, 2H), 2.35-2.23 (m, 7H), 2.18-1.97 (m, 4H), 1.95-1.86 (m, 1H), 1.82-1.65 (m, 5H), 1.60-1.53 (m, 1H), 1.16-1.03 (m, 2H), 0.64 (s, 1H), 0.41 (s, 1H). |
| 48 | 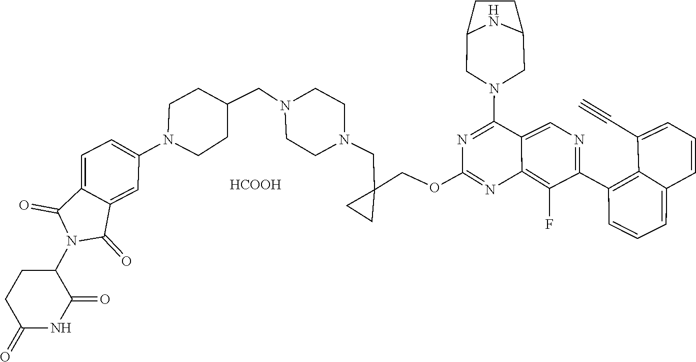 [SMILES: C#Cc1cccc2cccc(-c3ncc4c(N5CC6CCC(C5)N6)nc(OCC5(CN6CCN(CC7CCN(c8ccc9c(c8)C(=O)N(C8CCC(=O)NC8=O)C9=O)CC7)CC6)CC5)nc4c3F)c12 ; "HCOOH"] | 931.5 | 11.08 (s, 1H), 9.04 (s, 1H), 8.25 (s, 1H), 8.18-8.12 (m, 2H), 7.75-7.62 (m, 3H), 7.60-7.54 (m, 2H), 7.31-7.28 (m, 1H), 7.25-7.20 (m, 1H), 5.06 (dd, J = 5.4, 12.6 Hz, 1H), 4.50 (d, J = 10.4 Hz, 1H), 4.33-4.25 (m, 3H), 4.07-3.98 (m, 2H), 3.69-3.63 (m, 2H), 3.59-3.52 (m, 4H), 2.99-2.83 (m, 4H), 2.60 (d, J = 1.9 Hz, 2H), 2.57-2.55 (m, 2H), 2.32-2.24 (m, 4H), 2.13-1.97 (m, 3H), 1.81-1.63 (m, 7H), 1.28-1.04 (m, 3H), 0.64 (s, 2H), 0.41 (s, 2H). |
| 49 | 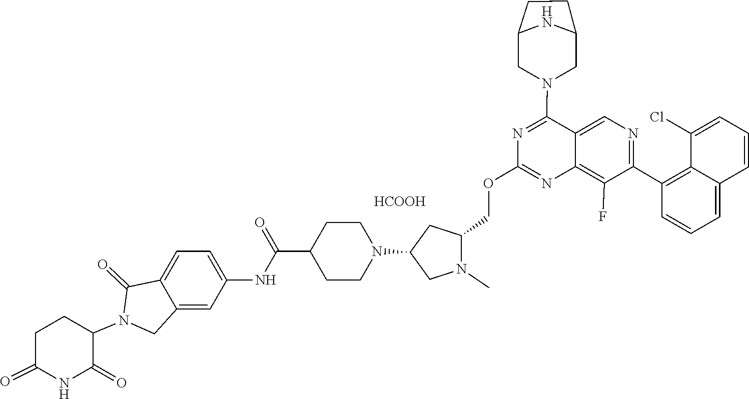 [SMILES: CN1C[C@H](N2CCC(C(=O)Nc3ccc4c(c3)CN(C3CCC(=O)NC3=O)C4=O)CC2)C[C@@H]1COc1nc(N2CC3CCC(C2)N3)c2cnc(-c3cccc4cccc(Cl)c34)c(F)c2n1 ; "HCOOH"] | 901.3 | 11.06-10.88 (m, 1H), 10.23-10.16 (m, 1H), 9.09 (s, 1H), 8.38-8.31 (m, 3H), 8.24-8.17 (m, 1H), 8.13-8.06 (m, 1H), 8.03-7.98 (m, 1H), 7.76-7.69 (m, 1H), 7.67-7.63 (m, 2H), 7.27-7.18 (m, 1H), 5.16-5.01 (m, 1H), 4.47-4.37 (m, 4H), 4.36-4.26 (m, 2H), 3.05-2.85 (m, 8H), 2.61 (d, J = 4.0 Hz, 2H), 2.32 (d, J = 7.8 Hz, 6H), 2.16-2.05 (m, 2H), 2.04- 1.93 (m, 4H), 1.81-1.75 (m, 2H), 1.64 (d, J = 4.0 Hz, 5H), 1.27-1.20 (m, 2H). |
| 50 | 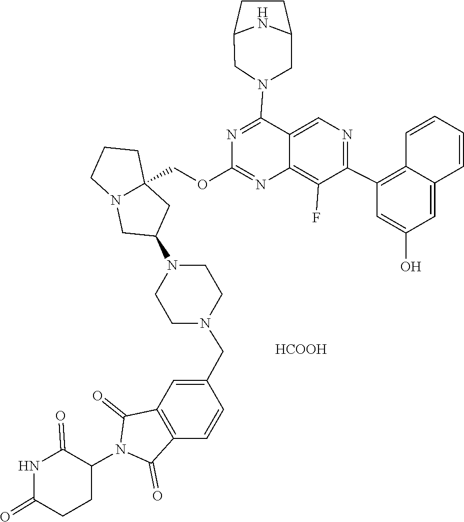 [SMILES: O=C1CCC(N2C(=O)c3ccc(CN4CCN([C@H]5CN6CCC[C@@]6(COc6nc(N7CC8CCC(C7)N8)c7cnc(-c8cc(O)cc9ccccc89)c(F)c7n6)C5)CC4)cc3C2=O)C(=O)N1 ; "HCOOH"] | 895.7 | 11.13 (s, 1H), 9.18 (s, 1H), 8.22 (s, 2H), 7.90-7.85 (m, 1H), 7.80 (d, J = 9.8 Hz, 2H), 7.55 (d, J = 8.4 Hz, 1H), 7.44 (t, J = 7.4 Hz, 1H), 7.29 (d, J = 2.0 Hz, 1H), 7.27-7.21 (m, 2H), 5.15 (dd, J = 5.4, 12.9 Hz, 1H), 4.50 (d, J = 10.6 Hz, 2H), 4.17-4.00 (m, 5H), 3.65-3.62 (m, 3H), 3.27-3.21 (m, 1H), 2.95-2.79 (m, 3H), 2.62 (d, J = 2.4 Hz, 1H), 2.58 (s, 1H), 2.55 (s, 1H), 2.53 (d, J = 1.8 Hz, 2H), 2.39 (d, J = 8.3 Hz, 4H), 2.34 (dd, J = 1.8, 3.7 Hz, 2H), 2.31-2.20 (m, 2H), 2.10-1.96 (m, 2H), 1.92-1.64 (m, 8H), 1.42 (t, J = 11.2 Hz, 1H). |
| 51 | 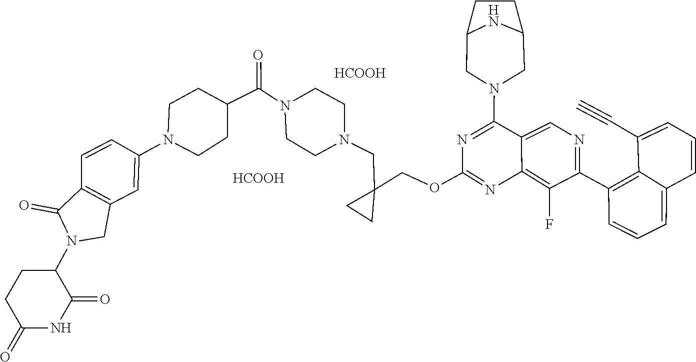 [SMILES: C#Cc1cccc2cccc(-c3ncc4c(N5CC6CCC(C5)N6)nc(OCC5(CN6CCN(C(=O)C7CCN(c8ccc9c(c8)CN(C8CCC(=O)NC8=O)C9=O)CC7)CC6)CC5)nc4c3F)c12 ; "HCOOH" ; "HCOOH"] | 931.7 | 10.95 (s, 1H), 9.05 (s, 1H), 8.24 (s, 1H), 8.14 (t, J = 7.8 Hz, 2H), 7.76-7.67 (m, 2H), 7.61-7.54 (m, 2H), 7.54-7.48 (m, 1H), 7.08-7.01 (m, 2H), 5.05 (dd, J = 5.0, 13.7 Hz, 1H), 4.53-4.46 (m, 1H), 4.35-4.29 (m, 4H), 4.23-4.17 (m, 1H), 3.91-3.85 (m, 2H), 3.69-3.65 (m, 2H), 3.63 (s, 1H), 3.57 (s, 3H), 3.55-3.50 (m, 4H), 2.94-2.85 (m, 4H), 2.69-2.66 (m, 3H), 2.46-2.42 (m, 2H), 2.34 (dd, J = 1.8, 3.9 Hz, 5H), 2.00-1.91 (m, 1H), 1.67 (s, 5H), 0.66 (s, 2H), 0.46-0.40 (m, 2H). |
| 52 | 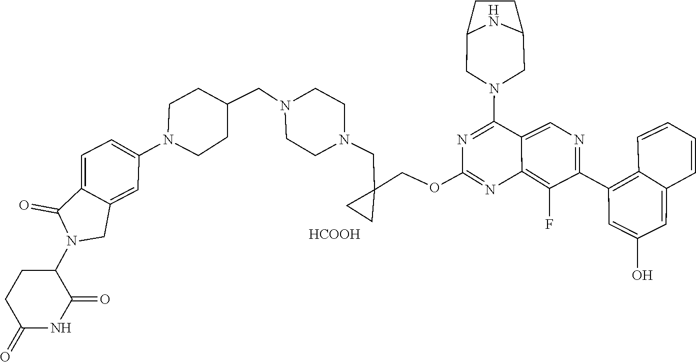 [SMILES: O=C1CCC(N2Cc3cc(N4CCC(CN5CCN(CC6(COc7nc(N8CC9CCC(C8)N9)c8cnc(-c9cc(O)cc%10ccccc9%10)c(F)c8n7)CC6)CC5)CC4)ccc3C2=O)C(=O)N1 ; "HCOOH"] | 909.6 | 10.97-10.92 (m, 1H), 9.16 (s, 1H), 8.27 (s, 1H), 7.83-7.77 (m, 1H), 7.56 (d, J = 7.8 Hz, 1H), 7.51-7.42 (m, 2H), 7.29 (d, J = 2.4 Hz, 1H), 7.25 (s, 1H), 7.23 (d, J = 1.8 Hz, 1H), 7.05-7.00 (m, 2H), 5.04 (dd, J = 4.8, 13.1 Hz, 1H), 4.46 (d, J = 11.6 Hz, 2H), 4.35-4.28 (m, 3H), 4.22- 4.16 (m, 1H), 3.84 (d, J = 11.4 Hz, 2H), 3.63 (s, 1H), 3.59 (s, 1H), 3.56 (s, 2H), 2.96-2.75 (m, 4H), 2.71-2.64 (m, 1H), 2.62-2.59 (m, 1H), 2.55 (d, J = 5.4 Hz, 1H), 2.43-2.36 (m, 3H), 2.34 (s, 1H), 2.30 (s, 3H), 2.26 (s, 1H), 2.07 (d, J = 6.4 Hz, 2H), 1.98-1.92 (m, 1H), 1.73 (d, J = 10.0 Hz, 3H), 1.65 (s, 4H), 1.19- 1.08 (m, 2H), 0.64 (s, 2H), 0.41 (s, 2H). |
| 53 | 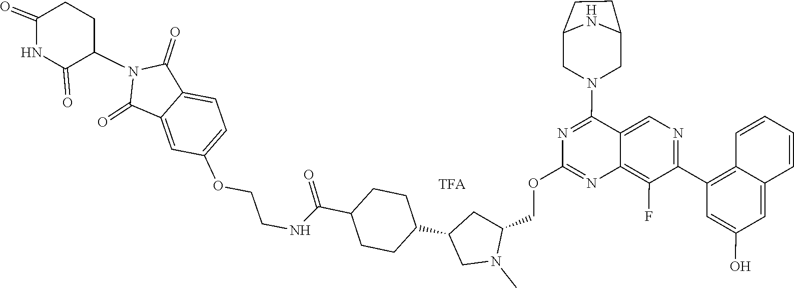 [SMILES: CN1C[C@H](C2CCC(C(=O)NCCOc3ccc4c(c3)C(=O)N(C3CCC(=O)NC3=O)C4=O)CC2)C[C@@H]1COc1nc(N2CC3CCC(C2)N3)c2cnc(-c3cc(O)cc4ccccc34)c(F)c2n1 ; "TFA"] | 941.3 | 11.12 (s, 1H), 10.15-9.98 (m, 1H), 9.44- 9.36 (m, 1H), 9.30-9.26 (m, 1H), 9.22- 9.11 (m, 1H), 8.30-8.19 (m, 1H), 7.88- 7.80 (m, 2H), 7.54 (d, J = 8.4 Hz, 1H), 7.49-7.42 (m, 2H), 7.37-7.30 (m, 2H), 7.28-7.23 (m, 2H), 5.17-5.06 (m, 1H), 4.74-4.62 (m, 4H), 4.21 (s, 5H), 3.94- 3.84 (m, 4H), 2.93-2.86 (m, 4H), 2.74 (s, 2H), 2.68 (d, J = 3.2 Hz, 1H), 2.60- 2.53 (m, 4H), 2.10-2.06 (m, 2H), 1.94 (s, 4H), 1.82-1.74 (m, 2H), 1.06 (t, J = 7.0 Hz, 3H). |
| 54 | 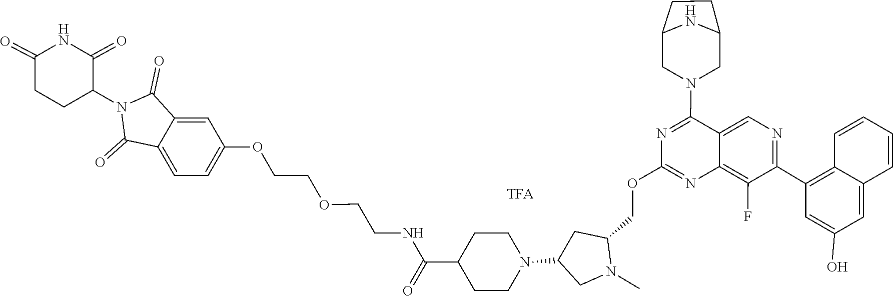 | 985.3 | 11.28 (s, 1H), 9.19-9.15 (m, 1H), 8.30 (s, 2H), 7.86-7.76 (m, 3H), 7.56 (d, J = 8.1 Hz, 1H), 7.46-7.41 (m, 2H), 7.39- 7.34 (m, 1H), 7.31-7.20 (m, 4H), 5.18- 5.07 (m, 1H), 4.50-4.43 (m, 3H), 4.35- 4.28 (m, 3H), 3.76 (s, 3H), 3.22-3.19 (m, 3H), 3.01-2.76 (m, 9H), 2.64-2.56 (m, 3H), 2.31 (s, 4H), 2.10-1.99 (m, 4H), 1.95 (s, 3H), 1.66 (s, 4H). |
| 55 | 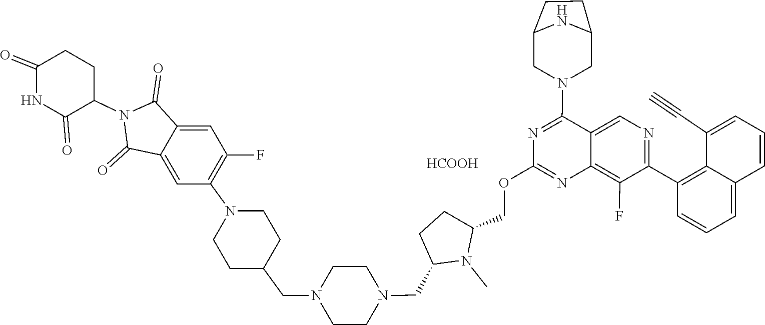 [SMILES: C#Cc1cccc2cccc(-c3ncc4c(N5CC6CCC(C5)N6)nc(OC[C@H]5CC[C@@H](CN6CCN(CC7CCN(c8cc9c(cc8F)C(=O)N(C8CCC(=O)NC8=O)C9=O)CC7)CC6)N5C)nc4c3F)c12 ; "HCOOH"] | 992.3 | 11.19-11.04 (m, 1H), 9.05 (s, 1H), 8.25 (s, 3H), 8.14 (t, J = 8.2 Hz, 2H), 7.75- 7.67 (m, 3H), 7.62-7.54 (m, 2H), 7.45- 7.40 (m, 1H), 5.20-5.02 (m, 1H), 4.54- 4.45 (m, 1H), 4.42-4.30 (m, 3H), 4.18- 4.10 (m, 2H), 3.67 (d, J = 2.5 Hz, 2H), 3.60-3.59 (m, 2H), 2.92-2.82 (m, 4H), 2.79-2.73 (m, 1H), 2.63-2.56 (m, 2H), 2.42-2.40 (m, 4H), 2.23-2.12 (m, 5H), 2.09-1.98 (m, 2H), 1.93-1.83 (m, 3H), 1.79 (d, J = 13.3 Hz, 3H), 1.68 (s, 5H), 1.64-1.43 (m, 4H), 1.32-1.16 (m, 5H). |
| 56 | 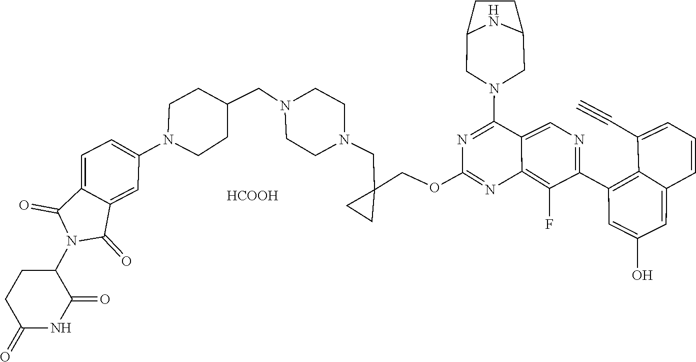 [SMILES: C#Cc1cccc2cc(O)cc(-c3ncc4c(N5CC6CCC(C5)N6)nc(OCC5(CN6CCN(CC7CCN(c8ccc9c(c8)C(=O)N(C8CCC(=O)NC8=O)C9=O)CC7)CC6)CC5)nc4c3F)c12 ; "HCOOH"] | 947.5 | 11.07 (s, 1H), 9.06-9.01 (m, 1H), 8.20 (s, 1H), 7.89 (d, J = 7.8 Hz, 1H), 7.65 (d, J = 8.4 Hz, 1H), 7.49-7.39 (m, 2H), 7.34 (d, J = 2.5 Hz, 1H), 7.29 (s, 1H), 7.22 (d, J = 8.4 Hz, 1H), 7.12 (d, J = 2.4 Hz, 1H), 5.06 (dd, J = 5.2, 13.3 Hz, 1H), 4.57-4.43 (m, 1H), 4.35-4.21 (m, 3H), 4.05-3.98 (m, 2H), 3.74-3.63 (m, 2H), 3.62-3.55 (m, 4H), 3.00-2.83 (m, 4H), 2.62-2.59 (m, 1H), 2.58-2.54 (m, 3H), 2.31-2.25 (m, 4H), 2.12-2.06 (m, 2H), 2.04-1.92 (m, 2H), 1.81-1.66 (m, 6H), 1.26-1.23 (m, 1H), 1.18-1.03 (m, 3H), 0.64 (s, 2H), 0.41 (s, 2H). |
| 57 | 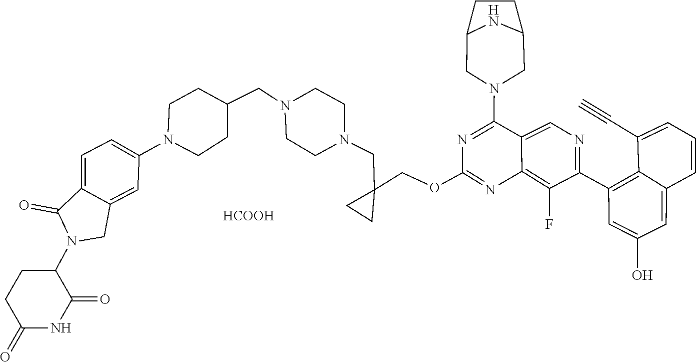 [SMILES: C#Cc1cccc2cc(O)cc(-c3ncc4c(N5CC6CCC(C5)N6)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 933.6 | 10.94 (s, 1H), 9.02 (s, 1H), 8.19 (s, 1H), 7.90-7.86 (m, 1H), 7.50-7.39 (m, 3H), 7.33 (d, J = 2.4 Hz, 1H), 7.10 (d, J = 2.4 Hz, 1H), 7.05-6.99 (m, 2H), 5.03 (dd, J = 5.0, 13.2 Hz, 1H), 4.56-4.47 (m, 1H), 4.34-4.23 (m, 4H), 4.20-4.15 (m, 1H), 3.85 (s, 1H), 3.83 (s, 1H), 3.68 (s, 1H), 3.66-3.60 (m, 3H), 3.59 (s, 1H), 3.54 (s, 1H), 2.94-2.87 (m, 1H), 2.87- 2.74 (m, 3H), 2.59 (s, 1H), 2.56 (d, J = 3.4 Hz, 1H), 2.36 (d, J = 4.6 Hz, 2H), 2.34-2.24 (m, 6H), 2.09 (d, J = 6.4 Hz, 2H), 1.94 (dd, J = 4.8, 9.8 Hz, 1H), 1.76-1.65 (m, 7H), 1.13 (d, J = 11.4 Hz, 2H), 0.68-0.58 (m, 2H), 0.43-0.36 (m, 2H). |
| 58 | 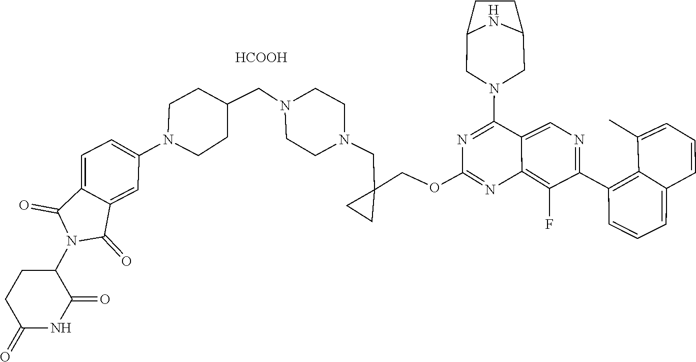 [SMILES: Cc1cccc2cccc(-c3ncc4c(N5CC6CCC(C5)N6)nc(OCC5(CN6CCN(CC7CCN(c8ccc9c(c8)C(=O)N(C8CCC(=O)NC8=O)C9=O)CC7)CC6)CC5)nc4c3F)c12 ; "HCOOH"] | 921.5 | 9.09 (s, 1H), 8.56-8.46 (m, 1H), 8.10- 8.02 (m, 1H), 7.89 (d, J = 8.1 Hz, 1H), 7.70-7.64 (m, 1H), 7.61-7.56 (m, 1H), 7.49-7.42 (m, 2H), 7.35 (s, 2H), 7.21 (dd, J = 1.8, 8.7 Hz, 1H), 5.13-5.04 (m, 2H), 4.77-4.64 (m, 3H), 4.52-4.41 (m, 2H), 4.03 (d, J = 12.4 Hz, 2H), 3.88- 3.73 (m, 5H), 3.55-3.49 (m, 1H), 3.24- 3.22 (m, 1H), 3.03-2.94 (m, 3H), 2.90- 2.82 (m, 3H), 2.78 (d, J = 2.6 Hz, 2H), 2.72-2.67 (m, 3H), 2.42-2.34 (m, 2H), 2.08-2.05 (m, 3H), 1.99-1.95 (m, 2H), 1.92-1.86 (m, 3H), 1.39-1.27 (m, 5H), 0.83-0.77 (m, 2H), 0.65-0.57 (m, 2H). |
| 59 | 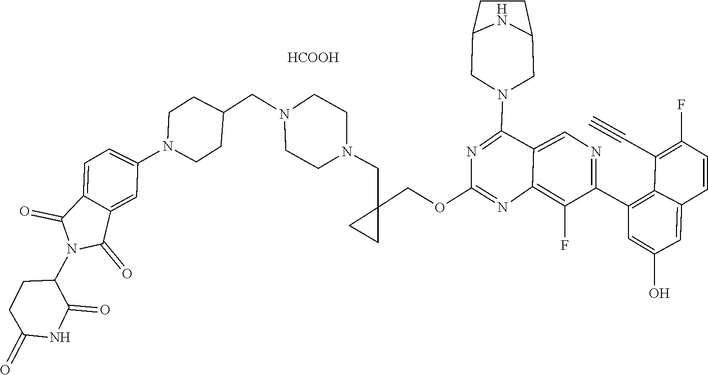 [SMILES: C#Cc1c(F)ccc2cc(O)cc(-c3ncc4c(N5CC6CCC(C5)N6)nc(OCC5(CN6CCN(CC7CCN(c8ccc9c(c8)C(=O)N(C8CCC(=O)NC8=O)C9=O)CC7)CC6)CC5)nc4c3F)c12 ; "HCOOH"] | 965.5 | 11.14-11.04 (m, 1H), 9.04 (s, 1H), 8.18 (s, 1H), 7.98 (dd, J = 6.0, 8.9 Hz, 1H), 7.65 (d, J = 8.6 Hz, 1H), 7.47 (t, J = 9.0 Hz, 1H), 7.40 (d, J = 2.0 Hz, 1H), 7.32- 7.27 (m, 1H), 7.26-7.15 (m, 2H), 5.11- 5.02 (m, 1H), 4.59-4.44 (m, 1H), 4.36- 4.20 (m, 3H), 4.06-3.90 (m, 3H), 3.69- 3.54 (m, 5H), 3.02-2.81 (m, 4H), 2.69- 2.59 (m, 2H), 2.36-2.24 (m, 7H), 2.11- 2.07 (m, 2H), 2.05-1.96 (m, 2H), 1.79- 1.67 (m, 7H), 1.30-1.21 (m, 2H), 1.15- 1.05 (m, 2H), 0.64 (s, 2H), 0.41 (s, 2H). |
| 60 | 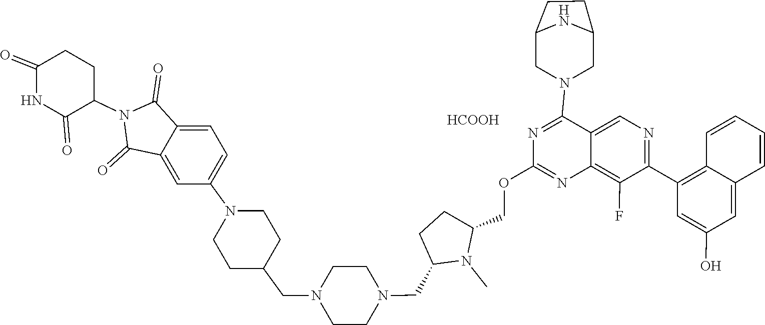 [SMILES: CN1[C@@H](COc2nc(N3CC4CCC(C3)N4)c3cnc(-c4cc(O)cc5ccccc45)c(F)c3n2)CC[C@H]1CN1CCN(CC2CCN(c3ccc4c(c3)C(=O)N(C3CCC(=O)NC3=O)C4=O)CC2)CC1 ; "HCOOH"] | 966.8 | 11.09 (s, 1H), 9.18 (s, 1H), 8.20 (s, 2H), 7.81 (d, J = 8.4 Hz, 1H), 7.65 (d, J = 8.4 Hz, 1H), 7.56 (d, J = 8.0 Hz, 1H), 7.44 (t, J = 7.4 Hz, 1H), 7.32-7.18 (m, 5H), 5.07 (dd, J = 5.6, 12.9 Hz, 1H), 4.54-4.35 (m, 4H), 4.20 (dd, J = 6.4, 10.3 Hz, 1H), 4.02 (d, J = 12.01 Hz, 2H), 3.68-3.61 (m, 5H), 2.99-2.84 (m, 4H), 2.79-2.70 (m, 1H), 2.69-2.60 (m, 3H), 2.43-2.39 (m, 5H), 2.26 (s, 2H), 2.18 (dd, J = 6.4, 12.1 Hz, 2H), 2.12- 2.08 (m, 2H), 2.06-1.97 (m, 2H), 1.93- 1.82 (m, 3H), 1.75 (d, J = 12.4 Hz, 3H), 1.68 (s, 3H), 1.63-1.56 (m, 1H), 1.53- 1.44 (m, 1H), 1.17-1.01 (m, 3H). |
| 61 | 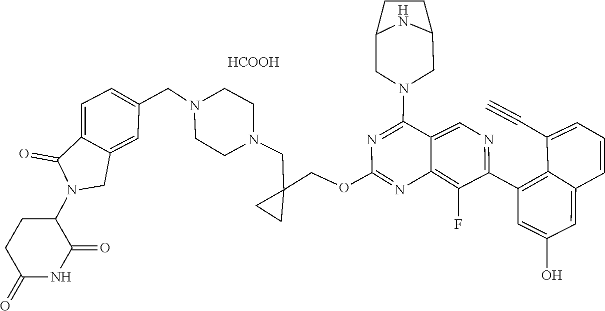 [SMILES: C#Cc1cccc2cc(O)cc(-c3ncc4c(N5CC6CCC(C5)N6)nc(OCC5(CN6CCN(Cc7ccc8c(c7)CN(C7CCC(=O)NC7=O)C8=O)CC6)CC5)nc4c3F)c12 ; "HCOOH"] | 850.3 | 11.16-11.08 (m, 1H), 8.98 (s, 1H), 8.24 (s, 3H), 7.98-7.90 (m, 1H), 7.88-7.86 (m, 1H), 7.82 (s, 1H), 7.77 (d, J = 7.8 Hz, 1H), 7.58 (d, J = 6.4 Hz, 1H), 7.52- 7.46 (m, 1H), 7.36 (d, J = 2.4 Hz, 1H), 7.30-7.26 (m, 1H), 5.16-5.12 (m, 1H), 4.42-4.34 (m, 2H), 4.30 (s, 2H), 3.60 (d, J = 14.6 Hz, 7H), 3.44 (q, J = 7.0 Hz, 4H), 2.94-2.88 (m, 1H), 2.69-2.59 (m, 1H), 2.34 (s, 3H), 2.28 (s, 3H), 2.08 (s, 2H), 1.66 (s, 1H), 1.62 (s, 1H), 1.06 (t, J = 7.0 Hz, 3H), 0.66 (s, 2H), 0.46-0.37 (m, 2H). |
| 62 | 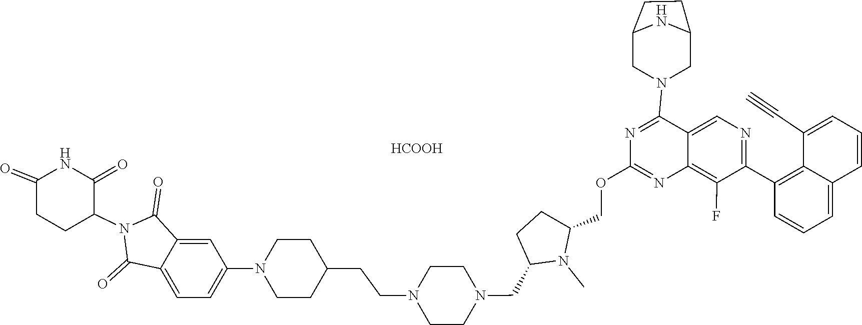 [SMILES: C#Cc1cccc2cccc(-c3ncc4c(N5CC6CCC(C5)N6)nc(OC[C@H]5CC[C@@H](CN6CCN(CCC7CCN(c8ccc9c(c8)C(=O)N(C8CCC(=O)NC8=O)C9=O)CC7)CC6)N5C)nc4c3F)c12 ; "HCOOH"] | 988.50 | 11.19-10.98 (m, 1H), 9.11 8.99 (m, 1H), 8.32-8.28 (m, 3H), 8.19-8.11 (m, 2H), 7.75-7.63 (m, 3H), 7.61-7.54 (m, 2H), 7.32-7.28 (m, 1H), 7.22 (d, J = 8.6 Hz, 1H), 5.07 (dd, J = 5.6, 12.9 Hz, 1H), 4.53-4.44 (m, 1H), 4.40-4.30 (m, 2H), 4.17-4.09 (m, 1H), 4.02 (d, J = 13.1 Hz, 3H), 3.69-3.66 (m, 2H), 3.57 (s, 5H), 2.99-2.83 (m, 4H), 2.78-2.70 (m, 1H), 2.62-2.58 (m, 3H), 2.41 (s, 3H), 2.31-2.25 (m, 4H), 2.17 (dd, J = 7.4, 2.1 Hz, 2H), 2.07-1.97 (m, 2H), 1.91-1.82 (m, 2H), 1.77-1.65 (m, 6H), 1.62-1.44 (m, 4H), 1.40-1.32 (m, 2H), 1.26-1.11 (m, 3H). |
| 63 | 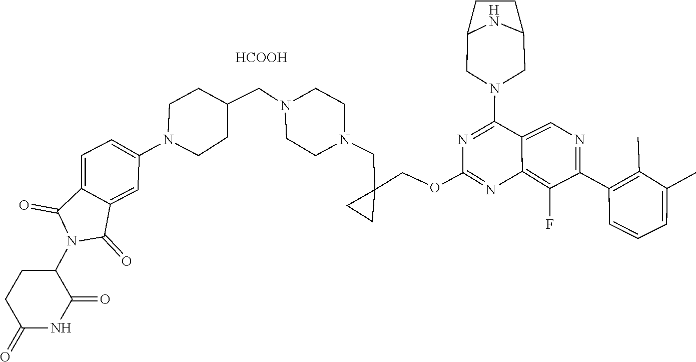 [SMILES: Cc1cccc(-c2ncc3c(N4CC5CCC(C4)N5)nc(OCC4(CN5CCN(CC6CCN(c7ccc8c(c7)C(=O)N(C7CCC(=O)NC7=O)C8=O)CC6)CC5)CC4)nc3c2F)c1C ; "HCOOH"] | 885.9 | 11.07 (s, 1H), 9.09 (s, 1H), 8.21 (s, 1H), 7.64 (d, J = 8.4 Hz, 1H), 7.34-7.15 (m, 5H), 5.06 (dd, J = 5.2, 12.8 Hz, 1H), 4.43 (d, J = 11.8 Hz, 2H), 4.29 (s, 2H), 4.01 (d, J = 12.1 Hz, 3H), 3.60 (d, J = 10.4 Hz, 6H), 3.03-2.81 (m, 5H), 2.34- 2.25 (m, 8H), 2.12-1.95 (m, 7H), 1.84- 1.60 (m, 8H), 1.18-1.05 (m, 2H), 0.63 (s, 2H), 0.40 (s, 2H). |
| 64 | 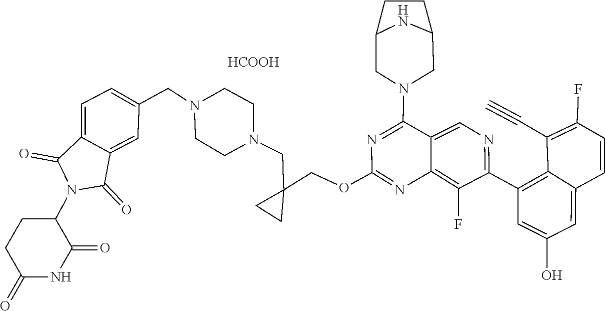 [SMILES: C#Cc1c(F)ccc2cc(O)cc(-c3ncc4c(N5CC6CCC(C5)N6)nc(OCC5(CN6CCN(Cc7ccc8c(c7)C(=O)N(C7CCC(=O)NC7=O)C8=O)CC6)CC5)nc4c3F)c12 ; "HCOOH"] | 882.4 | 11.13 (s, 1H), 9.04 (s, 1H), 8.21 (s, 2H), 7.98 (dd, J = 6.0, 9.0 Hz, 1H), 7.87 (d, J = 7.6 Hz, 1H), 7.82-7.75 (m, 2H), 7.47 (t, J = 9.0 Hz, 1H), 7.40 (d, J = 2.4 Hz, 1H), 7.18 (d, J = 2.4 Hz, 1H), 5.15 (dd, J = 5.2, 12.8 Hz, 1H), 4.51 (d, J = 12.6 Hz, 1H), 4.34-4.24 (m, 3H), 3.95 (s, 1H), 3.63 (s, 6H), 2.95-2.81 (m, 2H), 2.69-2.59 (m, 3H), 2.41-2.27 (m, 8H), 2.11-1.99 (m, 2H), 1.70 (s, 4H), 0.63 (s, 2H), 0.41 (s, 2H). |
| 65 | 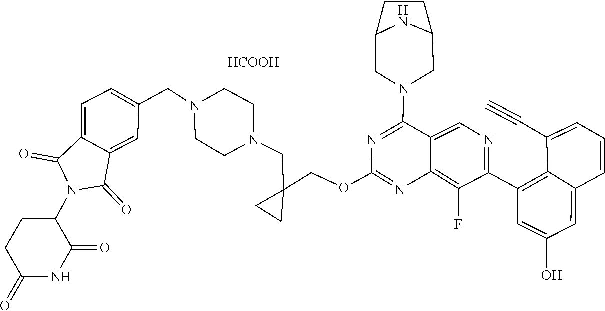 [SMILES: C#Cc1cccc2cc(O)cc(-c3ncc4c(N5CC6CCC(C5)N6)nc(OCC5(CN6CCN(Cc7ccc8c(c7)C(=O)N(C7CCC(=O)NC7=O)C8=O)CC6)CC5)nc4c3F)c12 ; "HCOOH"] | 864.5 | 11.25-11.00 (m, 1H), 9.00 (s, 1H), 8.27 (s, 1H), 7.91-7.85 (m, 2H), 7.82-7.77 (m, 2H), 7.48-7.39 (m, 2H), 7.34 (d, J = 2.4 Hz, 1H), 7.12 (d, J = 2.4 Hz, 1H), 5.15 (dd, J = 5.4, 13.0 Hz, 1H), 4.53- 4.46 (m, 1H), 4.26 (s, 2H), 3.63 (s, 3H), 3.59 (s, 1H), 3.57 (s, 3H), 3.53 (s, 1H), 2.95-2.82 (m, 2H), 2.69-2.67 (m, 1H), 2.62 (d, J = 2.0 H,z 1H), 2.58 (s, 1H), 2.43-2.36 (m, 5H), 2.35-2.33 (m, 2H), 2.32 (s, 2H), 1.72-1.61 (m, 4H), 0.63 (s, 2H), 0.41 (s, 2H). |
| 66 | 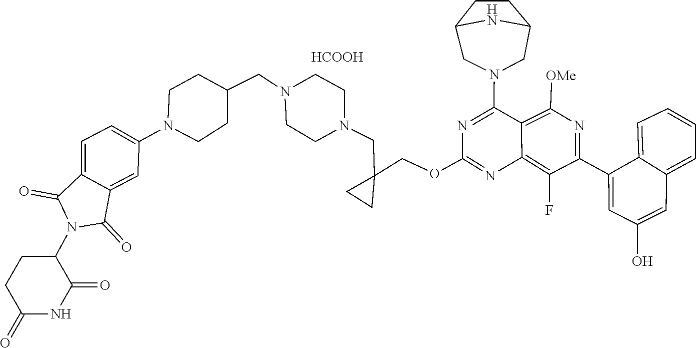 [SMILES: COc1nc(-c2cc(O)cc3ccccc23)c(F)c2nc(OCC3(CN4CCN(CC5CCN(c6ccc7c(c6)C(=O)N(C6CCC(=O)NC6=O)C7=O)CC5)CC4)CC3)nc(N3CC4CCC(C3)N4)c12 ; "HCOOH"] | 953.6 | 11.04 (s, 1H), 8.32-8.13 (m, 1H), 7.75 (d, J = 8.2 Hz, 1H), 7.68 (d, J = 8.0 Hz, 1H), 7.60 (d, J = 8.6 Hz, 1H), 7.42-7.37 (m, 1H), 7.25-7.21 (m, 4H), 7.18-7.15 (m, 1H), 5.02 (dd, J = 5.2, 12.8 Hz, 1H), 4.25 (s, 2H), 3.98 (s, 1H), 3.95 (s, 1H), 3.91 (s, 3H), 3.46 (s, 4H), 3.37 (s, 2H), 3.34 (s, 2H), 2.94-2.78 (m, 4H), 2.63 (dd, J = 1.8, 3.6 Hz, 1H), 2.29 (td, J = 1.8, 3.6 Hz, 2H), 2.27-2.22 (m, 4H), 2.05-1.95 (m, 4H), 1.69 (d, J = 10.8 Hz, 4H), 1.55 (s, 4H), 1.10-1.02 (m, 2H), 0.58 (s, 2H), 0.35 (s, 2H). |
| 67 | 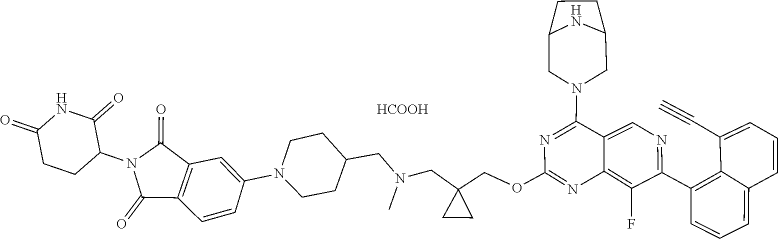 [SMILES: C#Cc1cccc2cccc(-c3ncc4c(N5CC6CCC(C5)N6)nc(OCC5(CN(C)CC6CCN(c7ccc8c(c7)C(=O)N(C7CCC(=O)NC7=O)C8=O)CC6)CC5)nc4c3F)c12 ; "HCOOH"] | 876.7 | 11.08 (s, 1H), 9.05 (s, 1H), 8.22 (s, 1H), 8.13 (t, J = 7.4 Hz, 2H), 7.76-7.64 (m, 2H), 7.59-7.49 (m, 3H), 7.17 (d, J = 1.8 Hz, 1H), 7.03 (d, J = 8.6 Hz, 1H), 5.09- 5.02 (m, 1H), 4.46 (d, J = 11.6 Hz, 1H), 4.37-4.29 (m, 2H), 4.18 (d, J = 10.4 Hz, 1H), 3.83 (t, J = 11.8 Hz, 3H), 3.57 (d, J = 11.0 Hz, 6H), 2.86-2.74 (m, 3H), 2.57-2.53 (m, 2H), 2.23-2.18 (m, 4H), 2.15 (d, J = 6.4 Hz, 2H), 2.03-1.96 (m, 1H), 1.72 (d, J = 11.4 Hz, 3H), 1.63 (s, 3H), 1.09-0.98 (m, 2H), 0.66-0.58 (m, 2H), 0.44-0.36 (m, 2H). |
| 68 | 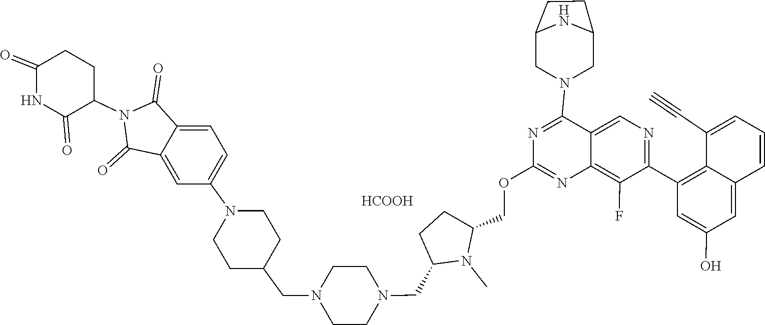 [SMILES: C#Cc1cccc2cc(O)cc(-c3ncc4c(N5CC6CCC(C5)N6)nc(OC[C@H]5CC[C@@H](CN6CCN(CC7CCN(c8ccc9c(c8)C(=O)N(C8CCC(=O)NC8=O)C9=O)CC7)CC6)N5C)nc4c3F)c12 ; "HCOOH"] | 991.7 | 11.19-0.95 (m, 1H), 9.09-8.97 (m, 1H), 8.19 (s, 2H), 7.89 (d, J = 8.5 Hz, 1H), 7.68-7.62 (m, 1H), 7.48-7.40 (m, 2H), 7.35 (d, J = 2.4 Hz, 1H), 7.30 (s, 1H), 7.22 (dd, J = 2.2, 8.8 Hz, 1H), 7.12 (d, J = 2.4 Hz, 1H), 5.10-5.02 (m, 1H), 4.56- 4.44 (m, 1H), 4.41-4.25 (m, 3H), 4.19- 4.11 (m, 1H), 4.02 (d, J = 13.4 Hz, 2H), 3.65 (t, J = 14.8 Hz, 5H), 3.59 (d, J = 2.6 Hz, 2H), 2.99-2.84 (m, 4H), 2.80- 2.72 (m, 1H), 2.63-2.55 (m, 2H), 2.42 (s, 5H), 2.26 (s, 1H), 2.23-2.16 (m, 2H), 2.11 (d, J = 7.0 Hz, 2H), 2.02 (dt, J = 5.0, 10.0 Hz, 2H), 1.96-1.83 (m, 3H), 1.80-1.69 (m, 6H), 1.64-1.54 (m, 2H), 1.52-1.42 (m, 1H), 1.17-1.07 (m, 2H). |
| 69 | 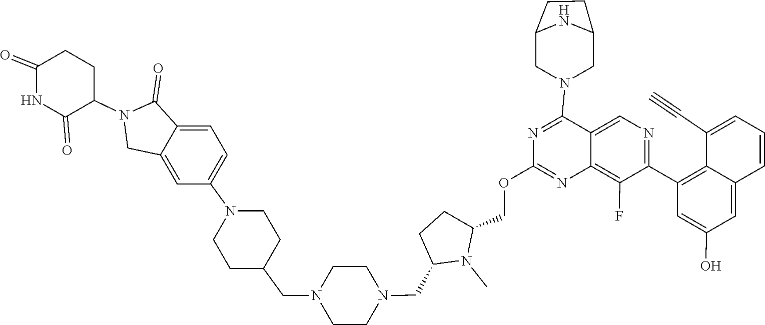 [SMILES: C#Cc1cccc2cc(O)cc(-c3ncc4c(N5CC6CCC(C5)N6)nc(OC[C@H]5CC[C@@H](CN6CCN(CC7CCN(c8ccc9c(c8)CN(C8CCC(=O)NC8=O)C9=O)CC7)CC6)N5C)nc4c3F)c12] | 976.8 | 10.93 (s, 1H), 9.04 (s, 1H), 8.19 (s, 2H), 7.88 (d, J = 7.8 Hz, 1H), 7.50-7.39 (m, 2H), 7.34 (d, J = 2.4 Hz, 1H), 7.12 (d, J = 2.4 Hz, 1H), 7.06-6.98 (m, 2H), 5.03 (dd, J = 5.0, 13.2 Hz, 1H), 4.64-4.42 (m, 2H), 4.40-4.28 (m, 3H), 4.23-4.08 (m, 3H), 3.84 (d, J = 12.6 Hz, 2H), 3.76-3.54 (m, 5H), 3.27 (s, 1H), 2.97- 2.70 (m, 4H), 2.62-2.51 (m, 3H), 2.45- 2.29 (m, 10H), 2.26 (s, 1H), 2.23-2.09 (m, 3H), 1.99-1.82 (m, 3H), 1.74 (s, 5H), 1.62-1.45 (m, 2H), 1.25-1.06 (m, 2H). |
| 70 | 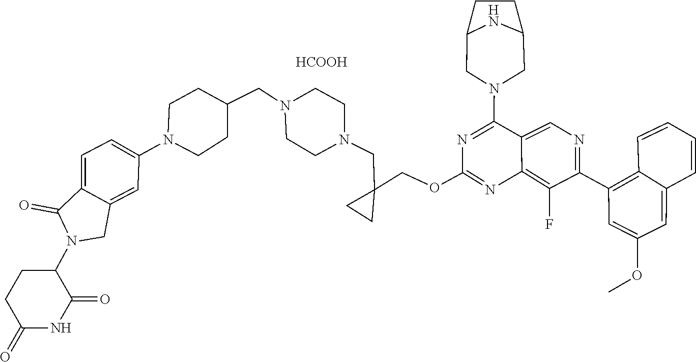 [SMILES: COc1cc(-c2ncc3c(N4CC5CCC(C4)N5)nc(OCC4(CN5CCN(CC6CCN(c7ccc8c(c7)CN(C7CCC(=O)NC7=O)C8=O)CC6)CC5)CC4)nc3c2F)c2ccccc2c1 ; "HCOOH"] | 923.8 | 10.93 (s, 1H), 9.17 (s, 1H), 8.19 (s, 1H), 7.94 (d, J = 8.3 Hz, 1H), 7.61 (d, J = 8.6 Hz, 1H), 7.55-7.46 (m, 3H), 7.39- 7.26 (m, 2H), 7.06-6.98 (m, 2H), 5.03 (dd, J = 5.0 Hz, 1H), 4.47 (d, J = 10.8 Hz, 2H), 4.34-4.27 (m, 3H), 4.21-4.16 (m, 1H), 3.94 (s, 3H), 3.83 (d, J = 12.4 Hz, 3H), 3.69-3.58 (m, 6H), 2.94-2.84 (m, 1H), 2.78 (t, J = 11.6 Hz, 2H), 2.62- 2.52 (m, 2H), 2.42-2.27 (m, 8H), 2.06 (d, J = 6.4 Hz, 2H), 1.98-1.91 (m, 1H), 1.77-1.63 (m, 7H), 1.20-1.06 (m, 2H), 0.64 (s, 2H), 0.41 (s, 2H). |
| 71 | 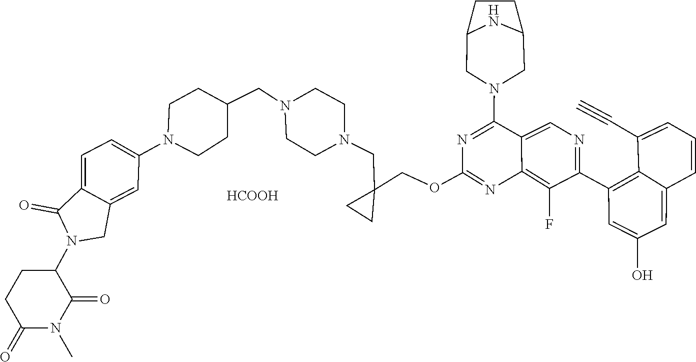 [SMILES: C#Cc1cccc2cc(O)cc(-c3ncc4c(N5CC6CCC(C5)N6)nc(OCC5(CN6CCN(CC7CCN(c8ccc9c(c8)CN(C8CCC(=O)N(C)C8=O)C9=O)CC7)CC6)CC5)nc4c3F)c12 ; "HCOOH"] | 948.6 | 10.23-10.11 (m, 1H), 9.11-9.05 (m, 1H), 8.14 (s, 1H), 7.89 (dd, J = 1.8, 7.9 Hz, 1H), 7.50 (d, J = 9.2 Hz, 1H), 7.47- 7.40 (m, 2H), 7.35 (d, J = 2.4 Hz, 1H), 7.13 (d, J = 2.4 Hz, 1H), 7.05-7.01 (m, 2H), 5.10 (dd, J = 5.0, 13.4 Hz, 1H), 4.65 (d, J = 12.9 Hz, 1H), 4.44 (d, J = 13.4 Hz, 1H), 4.34-4.27 (m, 3H), 4.20 (s, 1H), 4.12 (s, 2H), 3.89-3.78 (m, 4H), 3.62-3.58 (m, 1H), 3.32 (s, 8H), 2.99 (s, 3H), 2.83-2.75 (m, 3H), 2.72 (d, J = 2.4 Hz, 1H), 2.68-2.65 (m, 1H), 2.38 (d, J = 5.0 Hz, 2H), 2.35-2.30 (m, 2H), 2.21 (dd, J = 3.9, 5.6 Hz, 1H), 1.98-1.91 (m, 4H), 1.75 (d, J = 10.6 Hz, 3H), 1.16 (q, J = 11.0 Hz, 2H), 0.66 (s, 2H), 0.45 (s, 2H). |
| 72 | 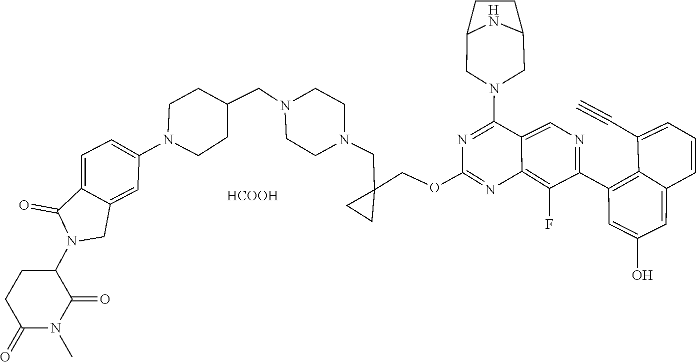 [SMILES: C#Cc1cccc2cc(O)cc(-c3ncc4c(N5CC6CCC(C5)N6)nc(OCC5(CN6CCN(CC7CCN(c8ccc9c(c8)CN(C8CCC(=O)N(C)C8=O)C9=O)CC7)CC6)CC5)nc4c3F)c12 ; "HCOOH"] | 961.4 | 9.03 (s, 1H), 8.20 (s, 2H), 7.89 (dd, J = 1.2, 7.8 Hz, 1H), 7.65 (d, J = 8.4 Hz, 1H), 7.49-7.39 (m, 2H), 7.36-7.29 (m, 2H), 7.25-7.20 (m, 1H), 7.12 (d, J = 2.4 Hz, 1H), 5.13 (dd, J = 5.0, 12.8 Hz, 1H), 4.51 (d, J = 11.2 Hz, 1H), 4.33- 4.22 (m, 3H), 4.08-3.94 (m, 3H), 3.70- 3.64 (m, 2H), 3.61-3.58 (m, 3H), 3.02 (s, 3H), 2.98 (s, 4H), 2.81-2.71 (m, 2H), 2.56 (d, J = 5.4 Hz, 2H), 2.45-2.36 (m, 4H), 2.32-2.26 (m, 4H), 2.14-1.99 (m 4H), 1.80-1.74 (m, 3H), 1.69 (s, 2H), 1.18-1.07 (m, 2H), 0.65 (s, 2H), 0.41 (s, 2H). |
| 73 | 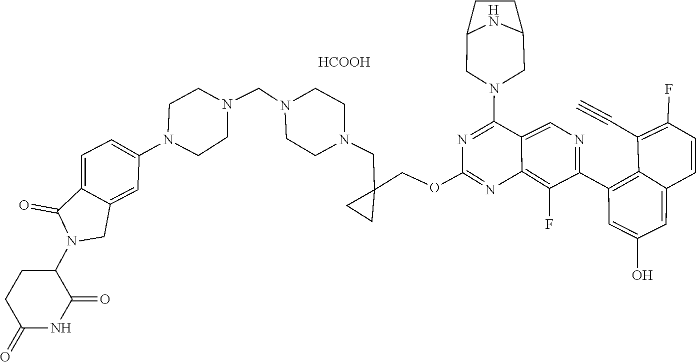 [SMILES: C#Cc1c(F)ccc2cc(O)cc(-c3ncc4c(N5CC6CCC(C5)N6)nc(OCC5(CN6CCN(CN7CCN(c8ccc9c(c8)CN(C8CCC(=O)NC8=O)C9=O)CC7)CC6)CC5)nc4c3F)c12 ; "HCOOH"] | 951.7 | 9.04 (s, 1H), 8.45 (s, 1H), 7.86 (dd, J = 5.9, 9.3 Hz, 1H), 7.61 (d, J = 8.2 Hz, 1H), 7.38-7.27 (m, 2H), 7.22 (d, J = 2.4 Hz, 1H), 7.08-7.01 (m, 2H), 5.10 (dd, J = 5.2, 13.4 Hz 1H), 4.69 (t, J = 14.0 Hz, 2H), 4.54 (d, J = 11.2 Hz, 1H), 4.41-4.37 (m, 2H), 3.97-3.84 (m, 4H), 3.81 (d, J = 14.0 Hz, 2H), 3.43-3.34 (m, 3H), 2.96-2.70 (m, 13H), 2.58-2.39 (m, 4H), 2.18-2.12 (m, 1H), 2.03-1.92 (m, 3H), 1.83 (d, J = 11.9 Hz, 3H), 1.35- 1.26 (m, 2H), 0.82 (s, 2H), 0.63 (s, 2H). |
| 74 | 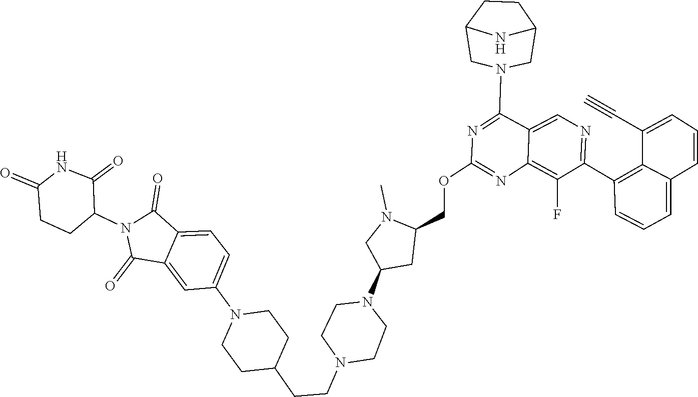 [SMILES: C#Cc1cccc2cccc(-c3ncc4c(N5CC6CCC(C5)N6)nc(OC[C@H]5C[C@@H](N6CCN(CCC7CCN(c8ccc9c(c8)C(=O)N(C8CCC(=O)NC8=O)C9=O)CC7)CC6)CN5C)nc4c3F)c12] | 974.4 | 11.07 (s, 1H), 9.05 (s, 1H), 8.21 (s, 2H), 8.14 (t, J = 7.6 Hz, 2H), 7.74-7.68 (m, 2H), 7.64 (d, J = 8.6 Hz, 1H), 7.61- 7.53 (m, 2H), 7.30-7.17 (m, 2H), 5.06 (dd, J = 5.3, 12.8 Hz, 1H), 4.52-4.40 (m, 2H), 4.37-4.29 (m, 2H), 4.02 (d, J = 11.6 Hz, 3H), 3.69-3.63 (m, 5H), 3.60 (s, 5H), 3.03-2.96 (m, 2H), 2.95-2.81 (m, 5H), 2.69-2.66 (m, 1H), 2.66-2.54 (m, 3H), 2.33 (td, J = 2.0, 3.8 Hz, 3H), 2.31 (s, 3H), 2.12-1.97 (m, 3H), 1.69 (s, 3H), 1.63-1.55 (m, 2H), 1.39-1.31 (m, 2H), 1.26-1.10 (m, 3H). |
| 75 | 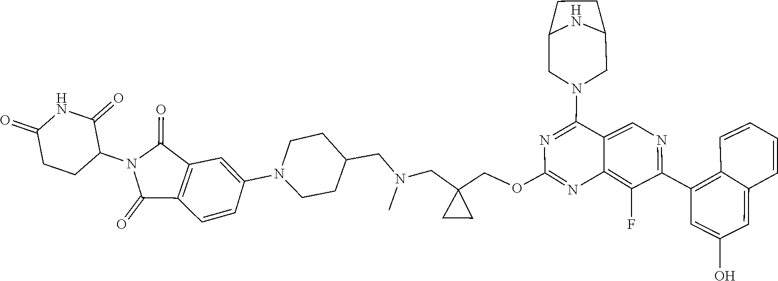 [SMILES: CN(CC1CCN(c2ccc3c(c2)C(=O)N(C2CCC(=O)NC2=O)C3=O)CC1)CC1(COc2nc(N3CC4CCC(C3)N4)c3cnc(-c4cc(O)cc5ccccc45)c(F)c3n2)CC1] | 868.3 | 11.07 (s, 1H), 9.18 (s, 1H), 8.20 (s, 1H), 7.79 (d, J = 8.4 Hz, 1H), 7.58-7.48 (m, 2H), 7.42 (t, J = 7.4 Hz, 1H), 7.28 (d, J = 2.1 Hz, 1H), 7.23-7.13 (m, 3H), 7.00 (dd, J = 2.1, 8.6 Hz, 1H), 5.04 (dd, J = 5.4, 12.8 Hz, 1H), 4.46 (d, J = 11.6 Hz, 2H), 4.29 (s, 2H), 3.84-3.78 (m, 2H), 3.60 (d, J = 11.8 Hz, 3H), 3.55 (s, 2H), 2.92-2.72 (m, 4H), 2.54 (s, 2H), 2.31 (s, 1H), 2.21 (s, 3H), 2.15 (d, J = 6.4 Hz, 2H), 2.02-1.94 (m, 1H), 1.71 (d, J = 12.0 Hz, 3H), 1.60 (s, 3H), 1.07-0.95 (m, 2H), 0.63 (s, 2H), 0.41 (s, 2H). |
| 76 | 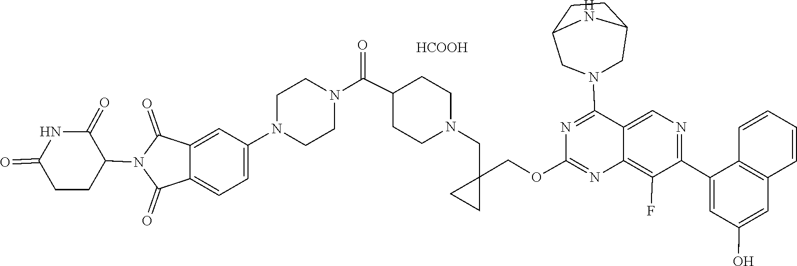 [SMILES: O=C1CCC(N2C(=O)c3ccc(N4CCN(C(=O)C5CCN(CC6(COc7nc(N8CC9CCC(C8)N9)c8cnc(-c9cc(O)cc%10ccccc9%10)c(F)c8n7)CC6)CC5)CC4)cc3C2=O)C(=O)N1 ; "HCOOH"] | 937.4 | 11.08 (s, 1H), 9.98 (s, 1H), 9.19 (s, 1H), 8.15 (s, 1H), 7.81 (d, J = 7.8 Hz, 1H), 7.74-7.69 (m, 1H), 7.56 (s, 1H), 7.47- 7.42 (m, 1H), 7.35 (d, J = 1.8 Hz, 1H), 7.29 (d, J = 2.0 Hz, 1H), 7.27-7.22 (m, 3H), 5.08 (dd, J = 5.4, 12.9 Hz, 1H), 4.57-4.50 (m, 2H), 4.36-4.29 (m, 2H), 3.82-3.74 (m, 2H), 3.71 (s, 1H), 3.67 (d, J = 6.8 Hz, 2H), 3.62-3.58 (m, 2H), 3.52-3.48 (m, 3H), 3.44 (d, J = 4.8 Hz, 5H), 3.08-2.96 (m, 3H), 2.95-2.83 (m, 2H), 2.70-2.66 (m, 2H), 2.34 (dd, J = 1.8, 3.6 Hz, 2H), 2.06-1.92 (m, 4H), 1.77 (s, 4H), 1.63-1.58 (m, 3H), 1.29- 1.22 (m, 2H), 0.72-0.63 (m, 2H), 0.49- 0.38 (m, 2H). |
| 77 | 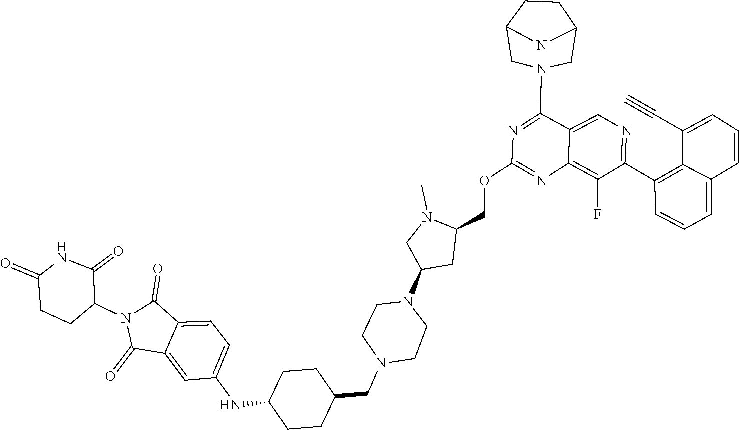 [SMILES: C#Cc1cccc2cccc(-c3ncc4c(N5CC6CCC(C5)N6)nc(OC[C@H]5C[C@@H](N6CCN(C[C@H]7CC[C@H](Nc8ccc9c(c8)C(=O)N(C8CCC(=O)NC8=O)C9=O)CC7)CC6)CN5C)nc4c3F)c12] | 974.4 | 11.09-11.02 (m, 1H), 9.05 (s, 1H), 8.20 (s, 1H), 8.17-8.12 (m, 2H), 7.76-7.66 (m, 2H), 7.63-7.50 (m, 3H), 7.02-6.93 (m, 2H), 6.88-6.81 (m, 1H), 5.02 (dd, J = 5.4, 12.4 Hz, 1H), 4.53-4.40 (m, 2H), 4.38-4.27 (m, 2H), 3.69-3.63 (m, 2H), 3.59 (s, 3H), 3.06-2.97 (m, 1H), 2.94- 2.81 (m, 2H), 2.69-2.65 (m, 2H), 2.47 (td, J = 2.0, 4.0 Hz, 3H), 2.34 (dd, J = 1.8, 3.6 Hz, 4H), 2.31 (s, 3H), 2.07 (d, J = 6.8 Hz, 3H), 2.03-1.94 (m, 4H), 1.85- 1.76 (m, 3H), 1.68 (s, 4H), 1.62-1.42 (m, 3H), 1.28-1.12 (m, 3H), 1.09-0.92 (m, 3H). |
| 78 | 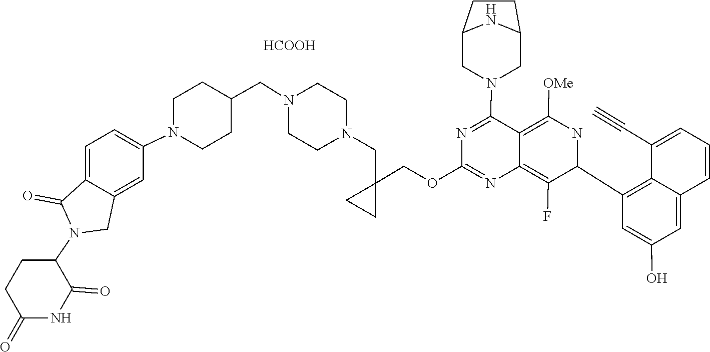 | 963.8 | 11.01 (s, 1H), 8.26-8.18 (m, 1H), 7.88 (d, J = 8.2 Hz, 1H), 7.52-7.46 (m, 2H), 7.45-7.39 (m, 1H), 7.36-7.30 (m, 1H), 7.17 (d, J = 2.2 Hz 1H), 7.07-7.00 (m, 2H), 5.04 (dd, J = 4.8, 13.3 Hz, 1H), 4.35-4.24 (m, 3H), 4.19 (d, J = 16.8 Hz, 2H), 3.88 (s, 3H), 3.84 (s, 2H), 3.55 (d, J = 9.2 Hz, 5H), 3.20 (d, J = 2.0 Hz, 2H), 2.86-2.75 (m, 3H), 2.55 (s, 3H), 2.32 (d, J = 12.4 Hz, 6H), 2.23 (s, 2H), 2.13-2.06 (m, 3H), 2.00-1.92 (m, 2H), 1.91-1.85 (m, 1H), 1.77-1.71 (m, 3H), 1.68-1.63 (m, 2H), 1.28-1.21 (m, 1H), 1.18-1.10 (m, 2H), 0.63 (s, 2H), 0.40 (s, 2H). |
| 79 | 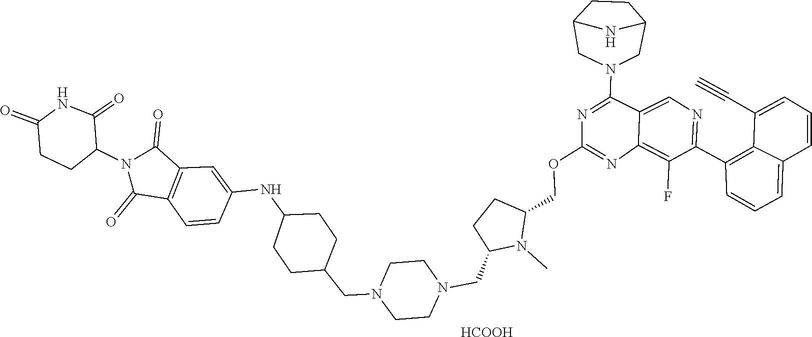 [SMILES: C#Cc1cccc2cccc(-c3ncc4c(N5CC6CCC(C5)N6)nc(OC[C@H]5CC[C@@H](CN6CCN(CC7CCC(Nc8ccc9c(c8)C(=O)N(C8CCC(=O)NC8=O)C9=O)CC7)CC6)N5C)nc4c3F)c12 ; "HCOOH"] | 988.6 | 11.06 (s, 1H), 9.10-9.00 (m, 1H), 8.20 (s, 1H), 8.15 (t, J = 8.0 Hz, 2H), 7.75- 7.66 (m, 2H), 7.62-7.50 (m, 3H), 6.99 (J = 7.8 Hz, 1H), 6.94 (s, 1H), 6.87- 6.82 (m, 1H), 5.09-4.97 (m, 1H), 4.49 (J = 11.8 Hz, 1H), 4.40-4.30 (m, 2H), 4.19-4.09 (m, 1H), 3.67 (d, J = 3.0 Hz, 1H), 3.65-3.56 (m, 6H), 3.47 (1H), 2.96-2.79 (m, 2H), 2.78-2.70 (m, 1H), 2.62-2.55 (m, 2H), 2.41 (s, 6H), 2.18 (J = 6.6, 11.9 Hz, 2H), 2.10-2.06 (m, 2H), 1.98 (J = 3.4, 10.8 Hz, 3H), 1.93-1.75 (m, 5H), 1.68 (4H), 1.63-1.53 (m, 2H), 1.52-1.41 (m, 2H), 1.24 (m, 2H), 1.09- 0.96 (m, 3H). |
| 80 | 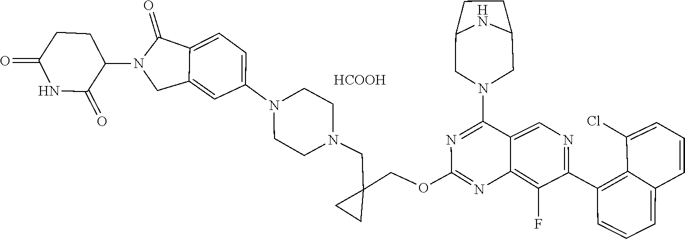 [SMILES: O=C1CCC(N2Cc3cc(N4CCN(CC5(COc6nc(N7CC8CCC(C7)N8)c7cnc(-c8cccc9cccc(Cl)c89)c(F)c7n6)CC5)CC4)ccc3C2=O)C(=O)N1 ; "HCOOH"] | 830.4 | 11.00-10.91 (m, 1H), 9.11-9.04 (m, 1H), 8.24 (s, 1H), 8.20-8.16 (m, 1H), 8.09 (d, J = 7.6 Hz, 1H), 7.72-7.65 (m, 1H), 7.63-7.49 (m, 4H), 7.05-6.98 (m, 2H), 5.14-4.97 (m, 1H), 4.49-4.16 (m, 7H), 3.64-3.54 (m, 5H), 3.22 (s, 5H), 2.98-2.85 (m, 1H), 2.61 (s, 2H), 2.43- 2.35 (m, 3H), 2.01-1.91 (m, 1H), 1.67- 1.60 (m, 4H), 0.68 (s, 2H), 0.48 (s, 2H). |
| 81 | 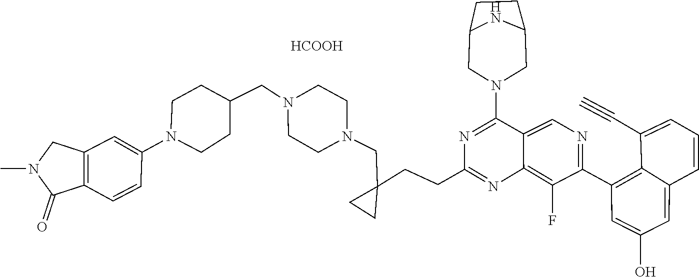 [SMILES: C#Cc1cccc2cc(O)cc(-c3ncc4c(N5CC6CCC(C5)N6)nc(CCC5(CN6CCN(CC7CCN(c8ccc9c(c8)CN(C)C9=O)CC7)CC6)CC5)nc4c3F)c12 ; "HCOOH"] | 836.4 | 9.03 (s, 1H), 8.20 (s, 1H), 7.89 (dd J = 1.6, 8.0 Hz, 1H), 7.49-7.39 (m, 3H), 7.34 (d, J = 2.6 Hz, 1H), 7.12 (d, J = 2.6 Hz, 1H), 7.04-6.96 (m, 2H), 4.55- 4.48 (m, 1H), 4.34-4.21 (m, 6H), 3.82 (d, J = 12.0 Hz, 3H), 3.67 (d, J = 12.8 Hz, 2H), 3.63 (s, 2H), 3.60-3.58 (m, 2H), 3.00 (s, 3H), 2.82-2.64 (m, 3H), 2.35-2.26 (m, 9H), 2.10 (d, J = 7.0 Hz, 2H), 1.77-1.68 (m, 6H), 1.18-1.10 (m, 2H), 0.64 (s, 2H), 0.41 (s, 2H). |
| 82 | 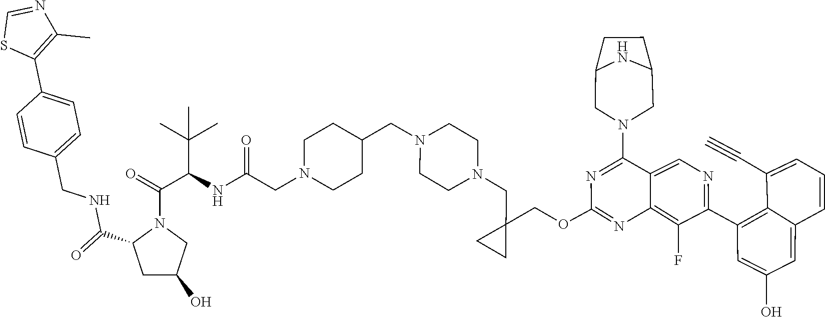 [SMILES: C#Cc1cccc2cc(O)cc(-c3ncc4c(N5CC6CCC(C5)N6)nc(OCC5(CN6CCN(CC7CCN(CC(=O)N[C@@H](C(=O)N8C[C@@H](O)C[C@@H]8C(=O)NCc8ccc(-c9scnc9C)cc8)C(C)(C)C)CC7)CC6)CC5)nc4c3F)c12] | 1162.3 | 10.16 (s, 1H), 9.02 (s, 1H), 8.99-8.96 (m, 1H), 8.60 (t, J = 6.0 Hz, 1H), 7.90- 7.87 (m, 1H), 7.79 (d, J = 9.4 Hz, 1H), 7.47-7.43 (m, 2H), 7.41 (d, J = 4.6 Hz, 4H), 7.34 (d, J = 2.4 Hz, 1H), 7.12 (d, J = 2.4 Hz, 1H), 5.17-5.09 (m, 1H), 4.50 (d, J = 9.6 Hz, 2H), 4.46-4.39 (m, 2H), 4.39-4.34 (m, 2H), 4.30-4.24 (m, 4H), 3.69-3.63 (m, 2H), 3.61 (s, 1H), 3.58 (s, 1H), 3.55 (s, 2H), 3.52 (s, 1H), 3.03- 2.96 (m, 1H), 2.89 (s, 1H), 2.82-2.75 (m, 2H), 2.69-2.66 (m, 1H), 2.44 (s, 3H), 2.35-2.32 (m, 2H), 2.26 (dd, J = 9.2, 12.6 Hz, 7H), 2.11-2.01 (m, 6H), 1.67 (s, 6H), 1.13-1.06 (m, 2H), 0.94 (s, 9H), 0.92 (s, 1H), 0.66-0.61 (m, 2H), 0.40 (s, 2H). |
| 83 | 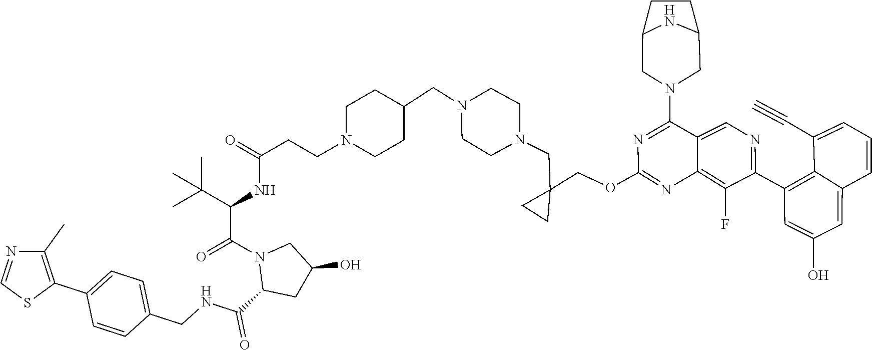 [SMILES: C#Cc1cccc2cc(O)cc(-c3ncc4c(N5CC6CCC(C5)N6)nc(OCC5(CN6CCN(CC7CCN(CCC(=O)N[C@@H](C(=O)N8C[C@@H](O)C[C@@H]8C(=O)NCc8ccc(-c9scnc9C)cc8)C(C)(C)C)CC7)CC6)CC5)nc4c3F)c12] | 1176.3 | 10.22-10.08 (m, 1H), 9.02 (s, 1H), 8.97 (s, 1H), 8.85 (d, J = 9.8 Hz, 1H), 8.63 (t, J = 5.9 Hz, 1H), 7.89 (d, J = 8.0 Hz 1H), 7.45 (d, J = 6.6 Hz, 3H), 7.43-7.40 (m, 1H), 7.38 (s, 1H), 7.36 (s, 1H), 7.34 (d, J = 2.4 Hz, 1H), 7.12 (d, J = 2.4 Hz, 1H), 5.16-5.10 (m, 1H), 4.55 (d, J = 9.8 Hz, 1H), 4.48 (s, 1H), 4.47-4.41 (m, 2H), 4.36 (s, 1H), 4.29 (s, 1H), 4.24 (s, 2H), 4.20 (d, J = 4.3 Hz, 1H), 3.66 (s, 1H), 3.63 (s, 1H), 3.58 (s, 1H), 3.54 (s, 2H), 2.96-2.84 (m, 2H), 2.70-2.66 (m, 1H), 2.44 (s, 3H), 2.40 (s, 2H), 2.35- 2.33 (m, 2H), 2.29-2.21 (m, 6H), 2.20- 2.16 (m, 2H), 2.08-1.98 (m, 4H), 1.96- 1.87 (m, 3H), 1.82 (d, J = 11.4 Hz, 1H), 1.66 (s, 6H), 1.51-1.35 (m, 2H), 1.26- 1.06 (m, 3H), 0.95 (s, 9H), 0.65-0.60 (m, 2H), 0.39 (s, 2H). |
| 84 | 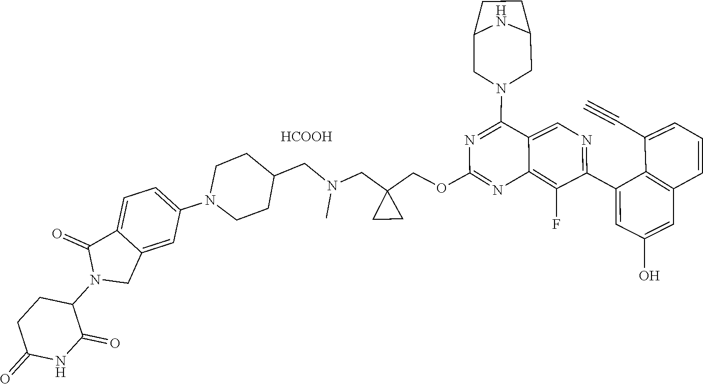 [SMILES: C#Cc1cccc2cc(O)cc(-c3ncc4c(N5CC6CCC(C5)N6)nc(OCC5(CN(C)CC6CCN(c7ccc8c(c7)CN(C7CCC(=O)NC7=O)C8=O)CC6)CC5)nc4c3F)c12 ; "HCOOH"] | 878.7 | 10.94 (s, 1H), 9.02 (d, J = 4.0 Hz, 1H), 8.21 (s, 1H), 7.88 (d, J = 8.0 Hz, 1H), 7.47-7.38 (m, 3H), 7.33 (s, 1H), 7.14- 7.09 (m, 1H), 6.88-6.79 (m, 2H), 5.05- 4.97 (m, 1H), 4.43 (dd, J = 3.8, 10.0 Hz, 2H), 4.32 (d, J = 14.4 Hz, 1H), 4.20-3.98 (m, 4H), 3.67-3.62 (m, 3H), 3.59 (d, J = 2.8 Hz, 3H), 3.52 (s, 1H), 2.98-2.80 (m, 2H), 2.69-2.62 (m, 2H), 2.54 (s, 2H), 2.25-2.13 (m, 8H), 1.90 (dd, J = 5.4, 10.2 Hz, 1H), 1.72-1.66 (m, 3H), 1.63 (s, 3H), 1.10-0.98 (m, 2H), 0.63 (s, 2H), 0.40 (d, J = 4.4 Hz, 2H). |
| 85 | 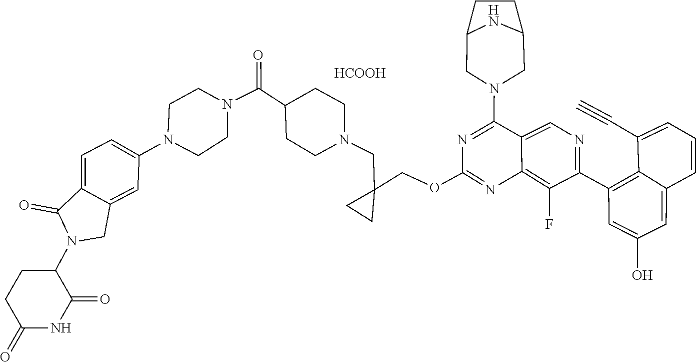 [SMILES: C#Cc1cccc2cc(O)cc(-c3ncc4c(N5CC6CCC(C5)N6)nc(OCC5(CN6CCC(C(=O)N7CCN(c8ccc9c(c8)CN(C8CCC(=O)NC8=O)C9=O)CC7)CC6)CC5)nc4c3F)c12 ; "HCOOH"] | 947.5 | 11.05-10.80 (m, 1H), 9.02 (s, 1H), 8.22 (s, 1H), 7.91-7.84 (m, 1H), 7.57-7.52 (m, 1H), 7.48-7.39 (m, 2H), 7.34 (d, J = 2.4 Hz, 1H), 7.13 (d, J = 2.6 Hz, 1H), 7.10-7.05 (m, 2H), 5.09-4.99 (m, 1H), 4.54-4.47 (m, 1H), 4.39-4.17 (m, 6H), 3.67-3.55 (m, 10H), 3.01-2.99 (m, 1H), 2.37 (dd, J = 3.4, 13.2 Hz, 3H), 2.30- 2.23 (m, 2H), 2.01-1.92 (m, 4H), 1.72- 1.64 (m, 5H), 1.62-1.55 (m, 4H), 1.29- 1.20 (m, 3H), 0.71-0.61 (m, 2H), 0.45- 0.36 (m, 2H). |
| 86 | 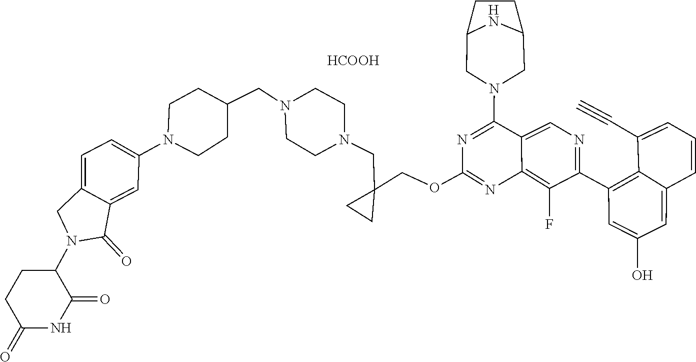 [SMILES: C#Cc1cccc2cc(O)cc(-c3ncc4c(N5CC6CCC(C5)N6)nc(OCC5(CN6CCN(CC7CCN(c8ccc9c(c8)C(=O)N(C8CCC(=O)NC8=O)C9)CC7)CC6)CC5)nc4c3F)c12 ; "HCOOH"] | 933.6 | 11.02-10.88 (m, 1H), 9.06-9.01 (m, 1H), 8.20 (s, 1H), 7.92-7.86 (m, 1H), 7.49-7.38 (m, 3H), 7.34 (d, J = 2.6 Hz, 1H), 7.27-7.21 (m, 1H), 7.15-7.10 (m, 2H), 5.14-5.03 (m, 1H), 4.57-4.44 (m, 1H), 4.39-4.14 (m, 6H), 3.76-3.63 (m, 6H), 3.61-3.56 (m, 5H), 2.97-2.85 (m, 2H), 2.70-2.63 (m, 4H), 2.30 (d, J = 9.6 Hz, 3H), 2.14-2.07 (m, 2H), 2.04-1.93 (m, 2H), 1.69 (s, 7H), 1.27-1.12 (m, 3H), 0.68-0.61 (m, 2H), 0.48-0.37 (m, 2H). |
| 87 | 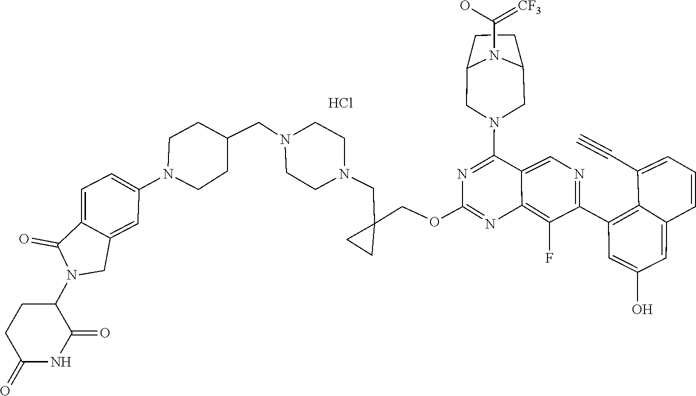 | 1029.9 | 10.94 (s, 1H), 10.18 (s, 1H), 9.06 (s, 1H), 8.13 (s, 1H), 7.89 (d, J = 7.0 Hz, 1H), 7.55-7.38 (m, 3H), 7.35 (d, J = 2.6 Hz, 1H), 7.12 (t, J = 2.4 Hz, 1H), 7.06- 7.00 (m, 2H), 5.04 (dd, J = 5.0, 13.2 Hz, 1H), 4.80 (s, 1H), 4.73 (d, J = 12.4 Hz, 1H), 4.64 (d, J = 5.9 Hz, 1H), 4.56- 4.47 (m, 1H), 4.35-4.26 (m, 3H), 4.23- 4.14 (m, 1H), 3.85 (d, J = 11.4 Hz, 2H), 3.76 (d, J = 13.4 Hz, 1H), 3.71-3.65 (m, 1H), 3.59 (d, J = 6.4 Hz, 1H), 2.95-2.76 (m, 5H), 2.56 (s, 3H), 2.47-2.29 (m, 8H), 2.05-1.85 (m, 6H), 1.74 (d, J = 11.8 Hz, 3H), 1.17 (d, J = 10.8 Hz, 2H), 0.69 (s, 2H), 0.47 (s, 2H). |
| 88 | 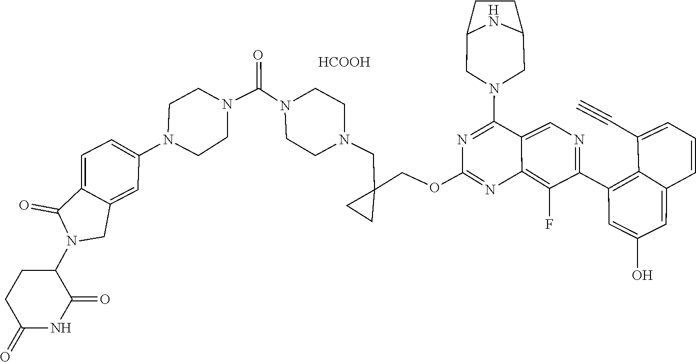 [SMILES: C#Cc1cccc2cc(O)cc(-c3ncc4c(N5CC6CCC(C5)N6)nc(OCC5(CN6CCN(C(=O)N7CCN(c8ccc9c(c8)CN(C8CCC(=O)NC8=O)C9=O)CC7)CC6)CC5)nc4c3F)c12 ; "HCOOH"] | 948.6 | 11.28 (s, 1H), 9.09-8.94 (m, 1H), 8.25 (s, 1H), 7.92-7.83 (m, 1H), 7.56-7.52 (m, 1H), 7.48-7.39 (m, 2H), 7.35-7.32 (m, 1H), 7.13-7.04 (m, 3H), 5.15-4.93 (m, 1H), 4.52-4.47 (m, 1H), 4.38-4.07 (m, 7H), 3.67-3.62 (m, 2H), 3.59-3.52 (m, 7H), 3.20-3.15 (m, 6H), 2.64 (s, 3H), 2.37-2.27 (m, 6H), 1.75-1.59 (m, 5H), 1.29-1.19 (m, 1H), 0.66 (s, 2H), 0.43 (s, 2H). |
| 89 | 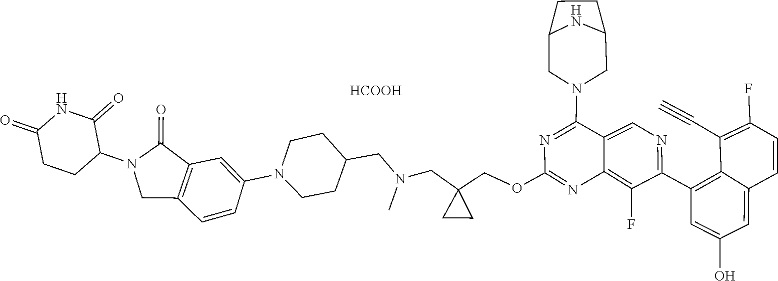 [SMILES: C#Cc1c(F)ccc2cc(O)cc(-c3ncc4c(N5CC6CCC(C5)N6)nc(OCC5(CN(C)CC6CCN(c7ccc8c(c7)C(=O)N(C7CCC(=O)NC7=O)C8)CC6)CC5)nc4c3F)c12 ; "HCOOH"] | 896.7 | 11.00 (s, 1H), 9.04 (d, J = 3.8 Hz, 1H), 8.20 (s, 1H), 8.02-7.93 (m, 1H), 7.46 (t, J = 9.0 Hz, 1H), 7.43-7.38 (m, 2H), 7.18 (t, J = 3.0 Hz, 1H), 6.89-6.79 (m, 2H), 5.06-4.97 (m, 1H), 4.47-4.39 (m, 2H), 4.37-4.31 (m, 1H), 4.20-4.04 (m, 3H), 3.90 (d, J = 1.8 Hz, 1H), 3.67 (dd, J = 7.0, 8.6 Hz, 3H), 3.58 (d, J = 11.8 Hz, 6H), 2.72-2.64 (m, 2H), 2.60 (s, 1H), 2.55 (d, J = 3.4 Hz, 1H), 2.35- 2.29 (m, 1H), 2.28-2.24 (m, 1H), 2.22- 2.18 (m, 3H), 2.15 (d, J = 4.9 Hz, 2H), 1.96-1.89 (m, 1H), 1.70 (dd, J = 1.4, 10.7 Hz, 3H), 1.64 (s, 4H), 1.12-0.97 (m, 2H), 0.66-0.60 (m, 2H), 0.45-0.36 (m, 2H). |
| 90 | 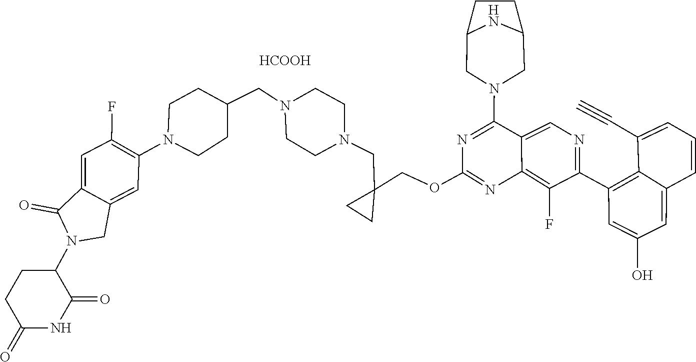 [SMILES: C#Cc1cccc2cc(O)cc(-c3ncc4c(N5CC6CCC(C5)N6)nc(OCC5(CN6CCN(CC7CCN(c8cc9c(cc8F)C(=O)N(C8CCC(=O)NC8=O)C9)CC7)CC6)CC5)nc4c3F)c12 ; "HCOOH"] | 951.7 | 10.97 (s, 1H), 9.02 (s, 1H), 8.19 (s, 2H), 7.88 (dd, J = 1.6, 8.1 Hz, 1H), 7.47- 7.38 (m, 2H), 7.33 (d, J = 2.6 Hz, 1H), 7.25-7.18 (m, 1H), 7.11 (d, J = 2.6 Hz, 1H), 5.06 (dd, J = 4.8, 13.3 Hz, 1H), 4.51 (d, J = 13.6 Hz, 1H), 4.38-4.19 (m, 5H), 3.69-3.63 (m, 3H), 3.61-3.58 (m, 3H), 3.54 (s, 2H), 2.94-2.86 (m, 2H), 2.75-2.66 (m, 3H), 2.62-2.56 (m, 2H), 2.38-2.28 (m, 8H), 2.13 (d, J = 6.8 Hz, 2H), 2.00-1.94 (m, 1H), 1.81-1.73 (m, 2H), 1.69 (s, 4H), 1.29-1.20 (m, 2H), 0.63 (s, 2H), 0.40 (s, 2H). |
| 91 | 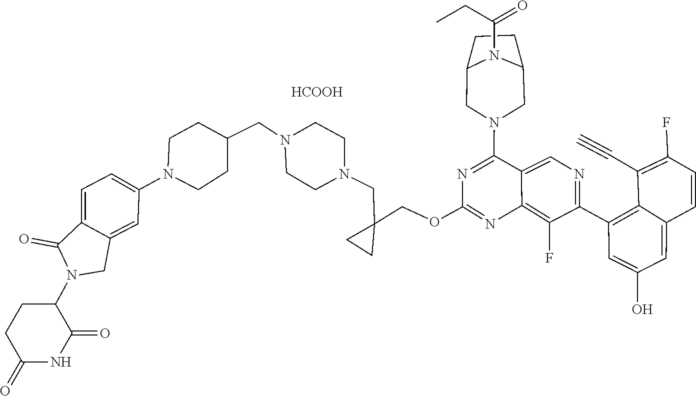 [SMILES: C#Cc1c(F)ccc2cc(O)cc(-c3ncc4c(N5CC6CCC(C5)N6C(=O)CC)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 1007.8 | 10.94 (s, 1H), 10.38-10.03 (m, 1H), 9.04 (d, J = 2.0 Hz, 1H), 8.20 (s, 1H), 7.98 (dd, J = 6.0 Hz, 1H), 7.50-7.44 (m, 2H), 7.39 (d, J = 2.4 Hz, 1H), 7.17 (s, 1H), 7.05-7.00 (m, 2H), 5.03 (dd, J = 13.2 Hz, 1H), 4.69 (s, 1H), 4.65-4.57 (m, 1H), 4.48 (s, 1H), 4.45-4.35 (m, 1H), 4.34-4.27 (m, 3H), 4.22-4.16 (m, 1H), 3.95 (d, J = 6.0 Hz, 1H), 3.89-3.76 (m, 3H), 3.70-3.58 (m, 2H), 2.92-2.71 (m, 4H), 2.62-2.54 (m, 2H), 2.44-2.37 (m, 5H), 2.34-2.28 (m, 5H), 2.09 (d, J = 6.8 Hz, 2H), 1.98-1.89 (m, 2H), 1.85- 1.70 (m, 6H), 1.20-1.10 (m, 2H), 1.06 (m, 3H), 0.64 (s, 2H), 0.41 (s, 2H). |
| 92 | 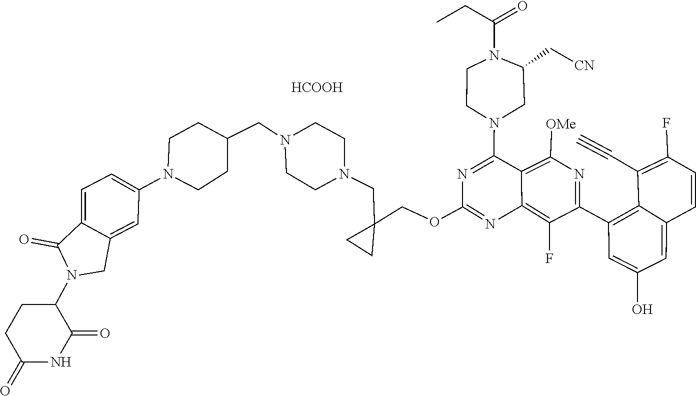 [SMILES: C#Cc1c(F)ccc2cc(O)cc(-c3nc(OC)c4c(N5CCN(C(=O)CC)[C@H](CC#N)C5)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 1050.48 | 10.96-10.92 (m, 1H), 10.24-10.15 (m, 1H), 8.28-8.22 (m, 1H), 8.02-7.94 (m, 1H), 7.53-7.45 (m, 2H), 7.42-7.38 (m, 1H), 7.26-7.21 (m, 1H), 7.07-7.00 (m, 2H), 5.06-5.02 (m, 1H), 4.36-4.27 (m, 3H), 4.25-4.15 (m, 2H), 4.07-3.99 (m, 1H), 3.96-3.92 (m, 2H), 3.92-3.88 (m, 2H), 3.85 (d, J = 12.8 Hz, 3H), 2.96- 2.86 (m, 3H), 2.85-2.73 (m, 3H), 2.70- 2.65 (m, 1H), 2.62-2.59 (m, 1H), 2.55 (d, 2H), 2.45-2.37 (m, 4H), 2.37-2.20 (m, 8H), 2.13-2.06 (m, 2H), 2.00-1.92 (m, 1H), 1.79-1.68 (m, 3H), 1.20-1.11 (m, 2H), 1.08-1.01 (m, 3H), 0.70 (s, 2H), 0.42 (s, 2H). |
| 93 | 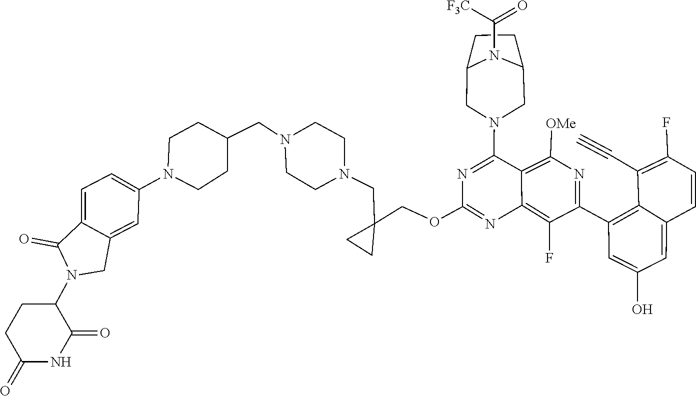 [SMILES: C#Cc1c(F)ccc2cc(O)cc(-c3nc(OC)c4c(N5CC6CCC(C5)N6C(=O)C(F)(F)F)nc(OCC5(CN6CCN(CC7CCN(c8ccc9c(c8)CN(C8CCC(=O)NC8=O)C9=O)CC7)CC6)CC5)nc4c3F)c12] | 1077.9 | 10.93 (s, 1H), 8.19-8.17 (m, 1H), 7.98 (dd, J = 5.8, 9.2 Hz, 1H), 7.52-7.45 (m, 2H), 7.39 (d, J = 2.4 Hz, 1H), 7.23 (s, 1H), 7.04-7.00 (m, 2H), 5.07-5.00 (m, 1H), 4.78-4.60 (m, 2H), 4.34-4.14 (m, 6H), 3.96 (s, 1H), 3.91 (s, 3H), 3.84 (d, J = 11.8 Hz, 3H), 2.94-2.83 (m, 2H), 2.79 (t, J = 11.8 Hz, 2H), 2.60 (s, 1H), 2.54 (s, 1H), 2.39-2.33 (m, 4H), 2.29 (d, J = 4.4 Hz, 4H), 2.08 (d, J = 6.6 Hz, 2H), 2.05-1.84 (m, 6H), 1.79-1.67 (m, 4H), 1.19-1.11 (m, 2H), 0.63 (s, 2H), 0.41 (s, 2H). |
| 94 | 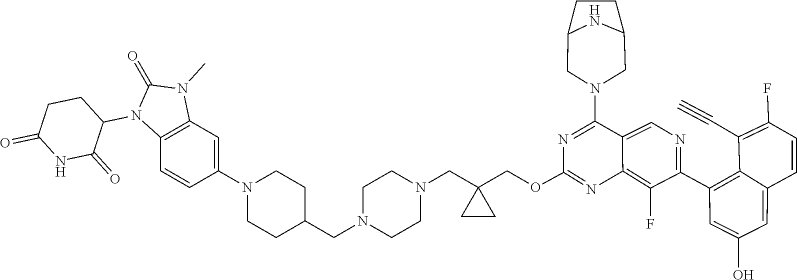 [SMILES: C#Cc1c(F)ccc2cc(O)cc(-c3ncc4c(N5CC6CCC(C5)N6)nc(OCC5(CN6CCN(CC7CCN(c8ccc9c(c8)n(C)c(=O)n9C8CCC(=O)NC8=O)CC7)CC6)CC5)nc4c3F)c12] | 948.5 | 11.15 (s, 1H), 10.40-9.94 (m, 1H), 9.17- 8.97 (m, 1H), 7.92-7.86 (m, 1H), 7.48- 7.38 (m, 2H), 7.34 (d, J = 2.4 Hz, 1H), 7.12 (d, J = 2.4 Hz, 1H), 6.92 (d, J = 8.6 Hz, 1H), 6.81 (d, J = 1.8 Hz, 1H), 6.64-6.60 (m, 1H), 5.33-5.22 (m, 1H), 4.27 (s, 2H), 3.62-3.51 (m, 5H), 3.30 (s, 7H), 2.94-2.85 (m, 1H), 2.66-2.54 (m, 5H), 2.32-2.24 (m, 6H), 2.12 (d, J = 6.6 Hz, 3H), 2.02-1.95 (m, 2H), 1.81-1.73 (m, 3H), 1.64-1.54 (m, 2H), 1.26-1.16 (m, 3H), 0.64 (s, 2H), 0.41 (s, 2H). |
| 95 | 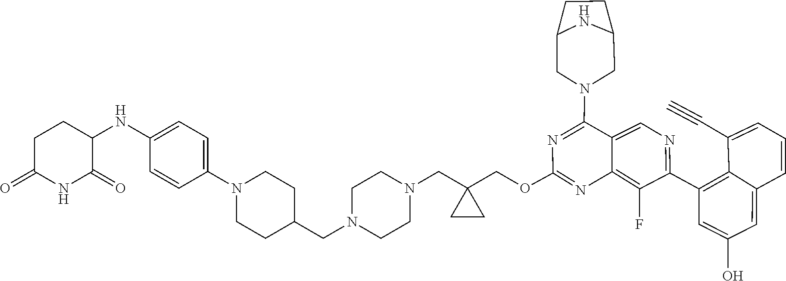 [SMILES: C#Cc1cccc2cc(O)cc(-c3ncc4c(N5CC6CCC(C5)N6)nc(OCC5(CN6CCN(CC7CCN(c8ccc(NC9CCC(=O)NC9=O)cc8)CC7)CC6)CC5)nc4c3F)c12] | 893.9 | 10.74 (s, 1H), 9.03 (s, 1H), 8.19 (s, 2H), 7.88 (dd, J = 1.4, 7.8 Hz, 1H), 7.49- 7.37 (m, 2H), 7.36-7.27 (m, 1H), 7.14- 7.06 (m, 1H), 6.73 (d, J = 8.8 Hz, 2H), 6.59 (d, J = 9.0 Hz, 2H), 5.43-5.21 (m, 1H), 4.59-4.39 (m, 1H), 4.29-4.24 (m, 2H), 4.17 (dd, J = 2.6 Hz, 1H), 3.73- 3.58 (m, 11H), 2.71-2.67 (m, 1H), 2.62- 2.55 (m, 2H), 2.35-2.25 (m, 7H), 2.15- 2.07 (m, 3H), 1.90-1.82 (m, 1H), 1.76- 1.70 (m, 5H), 1.58-1.49 (m, 1H), 1.28- 1.12 (m, 3H), 0.65 (s, 2H), 0.40 (s, 2H). |
| 96 | 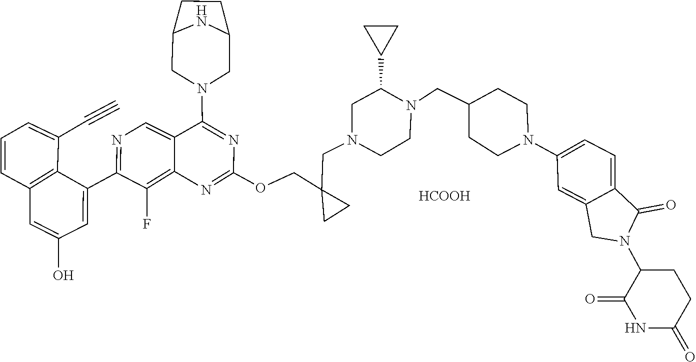 [SMILES: C#Cc1cccc2cc(O)cc(-c3ncc4c(N5CC6CCC(C5)N6)nc(OCC5(CN6CCN(CC7CCN(c8ccc9c(c8)CN(C8CCC(=O)NC8=O)C9=O)CC7)[C@@H](C7CC7)C6)CC5)nc4c3F)c12 ; "HCOOH"] | 974.0 | 10.95 (s, 1H), 9.03-8.97 (m, 1H), 8.21- 8.17 (m, 1H), 7.90-7.85 (m, 1H), 7.51- 7.38 (m, 3H), 7.33 (d, J = 2.4 Hz, 1H), 7.12-7.08 (m, 1H), 7.03-6.98 (m, 2H), 5.06-4.99 (m, 1H), 4.55-4.45 (m, 1H), 4.42-4.34 (m, 1H), 4.33-4.29 (m, 1H), 4.29-4.25 (m, 1H), 4.20 (s, 1H), 4.16 (t, J = 5.4 Hz, 1H), 3.87-3.80 (m, 2H), 3.69-3.59 (m, 2H), 3.58-3.52 (m, 4H), 2.97-2.68 (m, 9H), 2.15-1.80 (m, 9H), 1.66 (s, 4H), 1.29-1.05 (m, 4H), 0.67- 0.60 (m, 2H), 0.58-0.49 (m, 2H), 0.44- 0.35 (m, 2H), 0.33-0.26 (m, 1H), 0.17- 0.08 (m, 1H), 0.01-0.10 (m, 1H). |
| 97 | 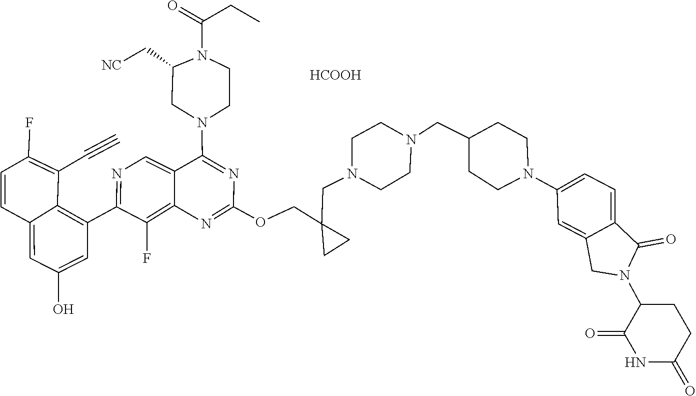 [SMILES: C#Cc1c(F)ccc2cc(O)cc(-c3ncc4c(N5CCN(C(=O)CC)[C@@H](CC#N)C5)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 1020.7 | 10.93 (s, 1H), 10.39-9.96 (m, 1H), 9.16- 9.04 (m, 1H), 8.21 (s, 1H), 7.98 (dd, J = 5.8, 9.3 Hz, 1H), 7.52-7.43 (m, 2H), 7.40 (d, J = 2.0 Hz, 1H), 7.21-7.15 (m, 1H), 7.05-6.99 (m, 2H), 5.03 (dd, J = 5.0 Hz, 1H), 4.87 (s, 1H), 4.44-4.14 (m, 7H), 4.02-3.75 (m, 6H), 3.05-2.73 (m, 7H), 2.63-2.54 (m, 3H), 2.37 (dd, J = 13.4 Hz, 5H), 2.30 (s, 3H), 2.11-2.07 (m, 2H), 1.98-1.93 (m, 1H), 1.77-1.68 (m, 3H), 1.30-1.09 (m, 3H), 1.06-0.99 (m, 3H), 0.64 (s, 2H), 0.42 (s, 2H). |
| 98 | 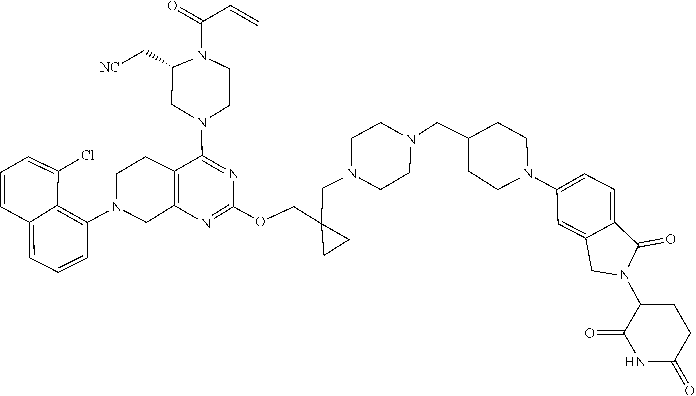 [SMILES: C=CC(=O)N1CCN(c2nc(OCC3(CN4CCN(CC5CCN(c6ccc7c(c6)CN(C6CCC(=O)NC6=O)C7=O)CC5)CC4)CC3)nc3c2CCN(c2cccc4cccc(Cl)c24)C3)C[C@@H]1CC#N] | 982.6 | 10.94 (s, 1H), 7.92 (d, J = 8.0 Hz, 1H), 7.74 (dd, J = 3.6 Hz, 1H), 7.60-7.42 (m, 4H), 7.38-7.29 (m, 1H), 7.06-7.00 (m, 2H), 5.04 (dd, J = 5.0 Hz, 1H), 4.35- 4.26 (m, 1H), 4.23-4.06 (m, 4H), 4.03- 3.66 (m, 6H), 3.58-3.43 (m, 1H), 3.21- 2.98 (m, 7H), 2.94-2.66 (m, 7H), 2.37- 1.90 (m, 12H), 1.80-1.71 (m, 3H), 1.27- 1.09 (m, 3H), 1.05-0.99 (m, 3H), 0.63- 0.51 (m, 2H), 0.43-0.31 (m, 2H). |
| 99 | 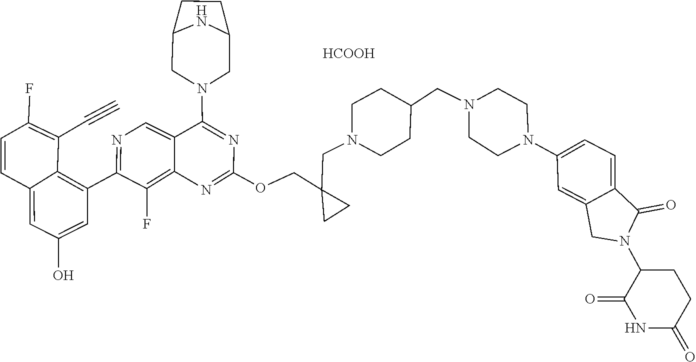 [SMILES: C#Cc1c(F)ccc2cc(O)cc(-c3ncc4c(N5CC6CCC(C5)N6)nc(OCC5(CN6CCC(CN7CCN(c8ccc9c(c8)CN(C8CCC(=O)NC8=O)C9=O)CC7)CC6)CC5)nc4c3F)c12 ; "HCOOH"] | 951.6 | 11.00-10.85 (m, 1H), 9.09-8.97 (m, 1H), 8.22 (s, 1H), 7.98 (dd, J = 6.0, 1H), 7.58-7.43 (m, 2H), 7.42-7.36 (m, 1H), 7.18 (d, J = 2.4 Hz, 1H), 7.09-6.97 (m, 2H), 5.10-4.95 (m, 1H), 4.58-4.43 (m, 1H), 4.38-4.14 (m, 5H), 2.31-2.27 (m, 3H), 2.16-2.08 (m, 3H), 2.00-1.84 (m, 4H), 1.71-1.63 (m, 6H), 1.28-1.22 (m, 2H), 1.16-0.95 (m, 3H), 0.64 (s, 2H), 0.45-0.33 (m, 2H). |
| 100 | 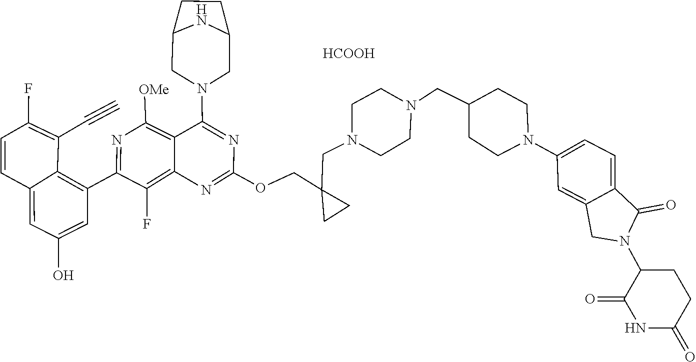 [SMILES: C#Cc1c(F)ccc2cc(O)cc(-c3nc(OC)c4c(N5CC6CCC(C5)N6)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 981.5 | — |
| 101 | 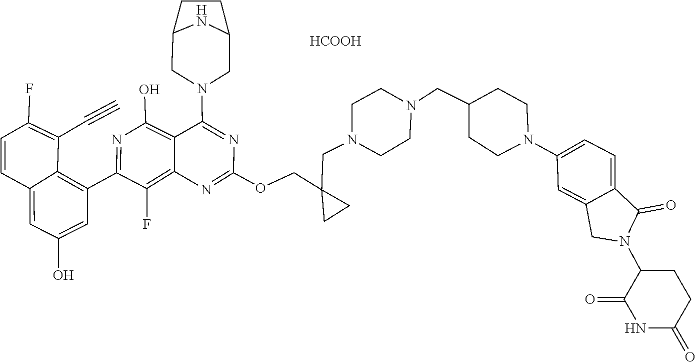 [SMILES: C#Cc1c(F)ccc2cc(O)cc(-c3nc(O)c4c(N5CC6CCC(C5)N6)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 967.5 | — |
| 102 | 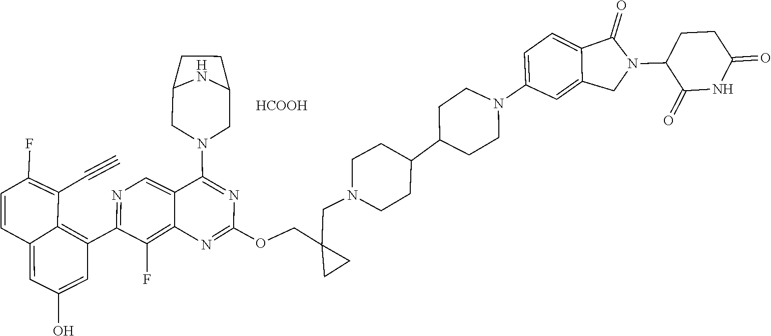 [SMILES: C#Cc1c(F)ccc2cc(O)cc(-c3ncc4c(N5CC6CCC(C5)N6)nc(OCC5(CN6CCC(C7CCN(c8ccc9c(c8)CN(C8CCC(=O)NC8=O)C9=O)CC7)CC6)CC5)nc4c3F)c12 ; "HCOOH"] | 936.4 | 10.96 (s, 1H), 9.04 (s, 1H), 8.19 (s, 1H), 8.01-7.96 (m, 1H), 7.52-7.44 (m, 2H), 7.40 (d, J = 2.4 Hz, 1H), 7.18 (d, J = 2.4 Hz, 1H), 7.03-6.98 (m, 2H), 5.10- 4.96 (m, 1H), 4.53-4.46 (m, 1H), 4.34- 4.26 (m, 4H), 4.22-4.14 (m, 1H), 3.95 (s, 1H), 3.87 (d, J = 11.8 Hz, 2H), 3.68- 3.57 (m, 5H), 3.04-2.96 (m, 3H), 2.93- 2.85 (m, 1H), 2.77-2.70 (m, 1H), 2.60 (d, J = 18.3 Hz, 1H), 2.38 (dd, J = 3.6, 4.6 Hz, 1H), 2.31-2.27 (m, 2H), 1.98- 1.92 (m, 1H), 1.84-1.76 (m, 2H), 1.69 (s, 5H), 1.63-1.58 (m, 2H), 1.24-1.05 (m, 6H), 0.67-0.61 (m, 2H), 0.40 (s, 2H). |
| 103 | 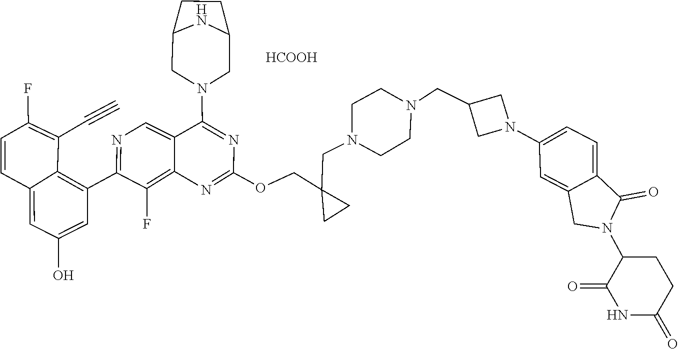 [SMILES: C#Cc1c(F)ccc2cc(O)cc(-c3ncc4c(N5CC6CCC(C5)N6)nc(OCC5(CN6CCN(CC7CN(c8ccc9c(c8)CN(C8CCC(=O)NC8=O)C9=O)C7)CC6)CC5)nc4c3F)c12 ; "HCOOH"] | 923.4 | 10.94 (s, 1H), 9.04 (s, 1H), 8.20 (s, 1H), 7.97 (dd, J = 6.0, 8.9 Hz, 1H), 7.50- 7.43 (m, 2H), 7.39 (d, J = 2.4 Hz, 1H), 7.17 (d, J = 2.4 Hz, 1H), 6.52-6.40 (m, 2H), 5.03 (dd, J = 5.0, 13.3 Hz, 1H), 4.51 (d, J = 12.0 Hz, 1H), 4.33-4.24 (m, 4H), 4.20-4.13 (m, 1H), 3.99 (t, J = 7.4 Hz, 2H), 3.94 (s, 1H), 3.66 (d, J = 13.0 Hz, 1H), 3.62-3.57 (m, 3H), 3.53 (dd, J = 5.8, 11.2 Hz, 5H), 2.97-2.84 (m, 3H), 2.62-2.58 (m, 1H), 2.55 (s, 2H), 2.41- 2.35 (m, 4H), 2.30 (d, J = 5.6 Hz, 2H), 2.26 (s, 1H), 1.99-1.91 (m, 1H), 1.69 (s, 4H), 0.69-0.59 (m, 2H), 0.45-0.37 (m, 2H). |
| 104 | 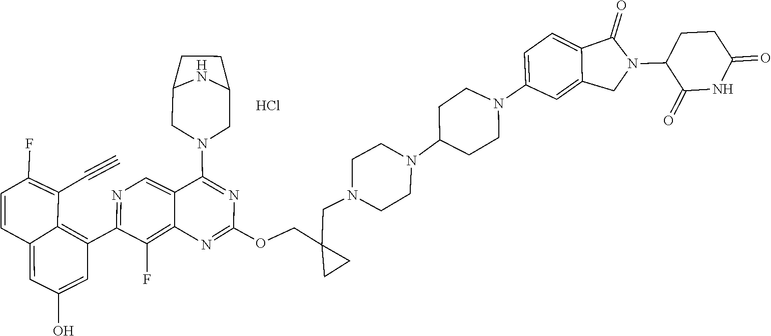 [SMILES: C#Cc1c(F)ccc2cc(O)cc(-c3ncc4c(N5CC6CCC(C5)N6)nc(OCC5(CN6CCN(C7CCN(c8ccc9c(c8)CN(C8CCC(=O)NC8=O)C9=O)CC7)CC6)CC5)nc4c3F)c12 ; Cl] | 937.6 | 10.97-10.92 (m, 1H), 10.30-10.20 (m, 1H), 9.75-9.53 (m, 1H), 9.39-9.23 (m, 1H), 9.17-9.07 (m, 1H), 8.04-7.93 (m, 1H), 7.55-7.45 (m, 2H), 7.43-7.39 (m, 1H), 7.22-7.17 (m, 1H), 7.07-7.04 (m, 1H), 5.08-5.02 (m, 1H), 4.68-4.60 (m, 1H), 4.58-4.47 (m, 1H), 4.40-4.30 (m, 2H), 4.28-4.16 (m, 4H), 4.06-3.81 (m, 6H), 3.10-3.05 (m, 1H), 2.94 (d, J = 5.0 Hz, 1H), 2.92-2.86 (m, 2H), 2.84-2.77 (m, 2H), 2.73 (s, 1H), 2.69-2.65 (m, 1H), 2.61 (s, 1H), 2.58-2.55 (m, 2H), 2.40-2.35 (m, 2H), 2.34-2.27 (m, 2H), 2.17-2.06 (m, 2H), 2.04-1.91 (m, 6H), 1.75-1.61 (m, 2H), 0.89-0.72 (m, 2H), 0.67-0.41 (m, 2H). |
| 105 | 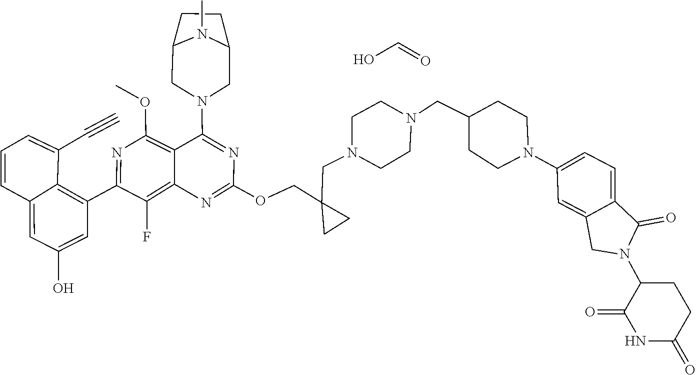 | 978.7 | 10.93 (s, 1H), 8.22 (s, 1H), 7.87 (d, J = 8.2 Hz, 1H), 7.51-7.46 (m, 2H), 7.44- 7.39 (m, 1H), 7.33 (d, J = 2.4 Hz, 1H), 7.16 (d, J = 2.4 Hz, 1H), 7.05-7.00 (m, 2H), 5.03 (dd, J = 5.0, 13.2 Hz, 1H), 4.34-4.28 (m, 1H), 4.26 (s, 2H), 4.21- 4.16 (m, 1H), 3.87 (s, 3H), 3.83 (s, 2H), 3.53 (s, 3H), 3.51 (s, 1H), 3.20 (s, 4H), 2.91-2.86 (m, 1H), 2.85-2.74 (m, 3H), 2.39 (dd, J = 4.6, 13.6 Hz, 4H), 2.34- 2.28 (m, 4H), 2.27 (s, 1H), 2.22 (s, 3H), 2.09 (d, J = 6.4 Hz, 2H), 1.97-1.92 (m, 1H), 1.87 (s, 2H), 1.74 (d, J = 11.8 Hz, 3H), 1.63-1.57 (m, 1H), 1.50-1.43 (m, 1H), 1.22-1.05 (m, 3H), 0.62 (s, 2H), 0.40 (s, 2H). |
| 106 | 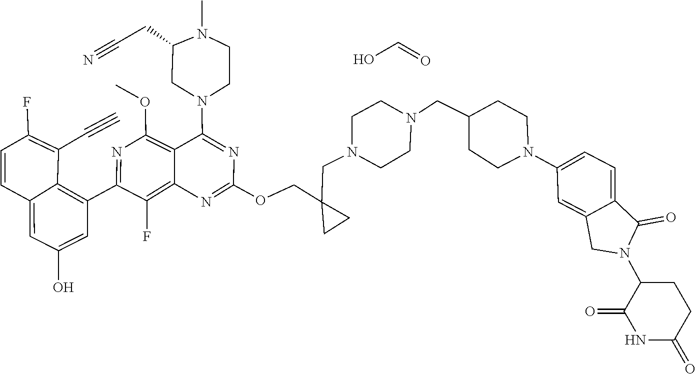 | 1008.7 | 10.93 (s, 1H), 8.16 (s, 1H), 7.97 (dd, J = 5.8, 9.2 Hz, 1H), 7.52-7.45 (m, 2H), 7.39 (d, J = 2.6 Hz, 1H), 7.22 (dd, J = 2.4, 5.4 Hz, 1H), 7.06-6.98 (m, 2H), 5.03 (dd, J = 5.2, 13.3 Hz, 1H), 4.35- 4.30 (m, 1H), 4.28 (d, J = 4.0 Hz, 2H), 4.18 (d, J = 17.0 Hz, 1H), 4.14-4.06 (m, 1H), 4.02 (d, J = 1.8 Hz, 1H), 3.95 (s, 1H), 3.90 (d, J = 4.6 Hz, 3H), 3.84 (d, J = 11.6 Hz, 2H), 3.21-3.07 (m, 4H), 2.95-2.87 (m, 4H), 2.86-2.74 (m, 3H), 2.60 (d, J = 2.8 Hz, 1H), 2.54 (s, 2H), 2.44-2.36 (m, 5H), 2.35-2.32 (m, 2H), 2.31 (d, J = 1.4 Hz, 3H), 2.30-2.29 (m, 1H), 2.09 (d, J = 3.8 Hz, 2H), 1.98-1.92 (m, 1H), 1.77-1.68 (m, 3H), 1.18-1.09 (m, 2H), 0.63 (s, 2H), 0.41 (s, 2H) |
| 107 | 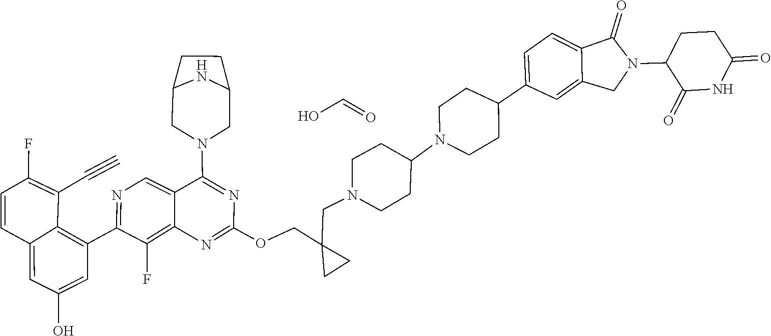 | 936.6 | 11.04-10.86 (m, 1H), 9.04 (s, 1H), 8.20 (s, 2H), 7.97 (dd, J = 5.8, 9.0 Hz, 1H), 7.63 (d, J = 7.8 Hz, 1H), 7.48-7.43 (m, 2H), 7.39-7.35 (m, 2H), 7.17 (d, J = 2.4 Hz, 1H), 5.10 (dd, J = 5.0, 13.2 Hz, 1H), 4.52 (d, J = 11.6 Hz, 1H), 4.44- 4.38 (m, 1H), 4.36-4.24 (m, 5H), 3.94 (s, 1H), 3.60 (d, J = 12.0 Hz, 2H), 3.06- 2.85 (m, 6H), 2.55 (d, J = 12.0 Hz, 2H), 2.39 (dd, J = 4.0, 12.8 Hz, 1H), 2.30 (s, 4H), 2.03-1.94 (m, 1H), 1.86 (t, J = 11.3 Hz, 2H), 1.80-1.60 (m, 11H), 1.50- 1.36 (m, 2H), 0.65 (s, 2H), 0.41 (s, 2H) |
| 108 | 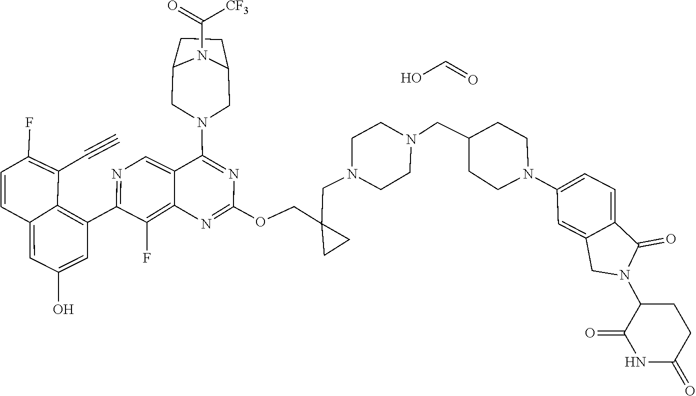 [SMILES: C#Cc1c(F)ccc2cc(O)cc(-c3ncc4c(N5CC6CCC(C5)N6C(=O)C(F)(F)F)nc(OCC5(CN6CCN(CC7CCN(c8ccc9c(c8)CN(C8CCC(=O)NC8=O)C9=O)CC7)CC6)CC5)nc4c3F)c12 ; O=CO] | 1047.3 | 10.93 (s, 1H), 9.05 (s, 1H), 8.15 (s, 1H), 7.98 (dd, J = 5.8, 9.2 Hz, 1H), 7.51- 7.43 (m, 2H), 7.40 (d, J = 2.4 Hz, 1H), 7.18 (s, 1H), 7.05-6.98 (m, 2H), 5.03 (dd, J = 5.2, 13.4 Hz, 1H), 4.82-4.70 (m, 2H), 4.65 (d, J = 6.0 Hz, 1H), 4.51 (d, J = 12.2 Hz, 1H), 4.34-4.27 (m, 3H), 4.21-4.15 (m, 1H), 3.93 (d, J = 6.0 Hz, 1H), 3.84 (d, J = 11.8 Hz, 2H), 3.76 (d, J = 12.8 Hz, 1H), 3.67 (d, J = 13.6 Hz, 1H), 2.95-2.84 (m, 2H), 2.82-2.75 (m, 2H), 2.60 (s, 2H), 2.40-2.26 (m, 8H), 2.09 (d, J = 6.4 Hz, 2H), 2.01-1.86 (m, 5H), 1.73 (d, J = 11.0 Hz, 3H), 1.20- 1.08 (m, 2H), 0.64 (s, 2H), 0.42 (s, 2H) |
| 109 | 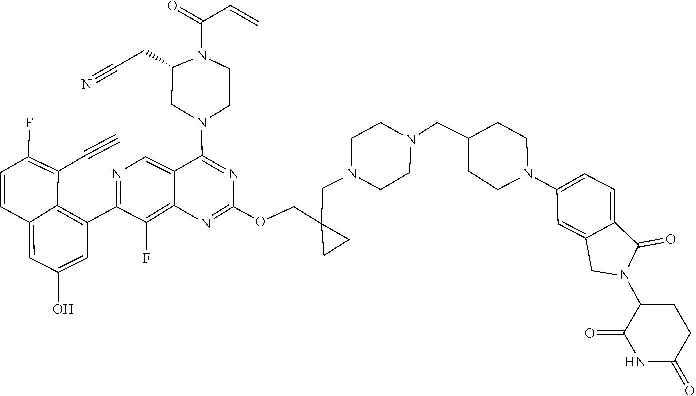 [SMILES: C#Cc1c(F)ccc2cc(O)cc(-c3ncc4c(N5CCN(C(=O)C=C)[C@@H](CC#N)C5)nc(OCC5(CN6CCN(CC7CCN(c8ccc9c(c8)CN(C8CCC(=O)NC8=O)C9=O)CC7)CC6)CC5)nc4c3F)c12] | 1018.6 | δ 10.95 (s, 1H), 10.36-10.04 (m, 1H), 9.17-9.09 (m, 1H), 8.21 (s, 1H), 8.03- 7.95 (m, 1H), 7.51-7.44 (m, 2H), 7.41 (d, J = 2.4 Hz, 1H), 7.20 (dd, J = 2.4, 5.6 Hz, 1H), 7.07-6.99 (m, 2H), 6.27- 6.18 (m, 1H), 5.87-5.76 (m, 1H), 5.10- 4.98 (m, 1H), 4.48-4.26 (m, 6H), 4.22- 4.15 (m, 1H), 3.98-3.92 (m, 1H), 3.88- 3.81 (m, 3H), 3.20-3.16 (m, 2H), 3.12- 3.04 (m, 3H), 2.96-2.75 (m, 6H), 2.58 (dd, J = 2.8, 19.8 Hz, 2H), 2.42-2.36 (m, 3H), 2.31 (s, 2H), 2.14-2.06 (m, 2H), 2.00-1.89 (m, 2H), 1.78-1.70 (m, 3H), 1.26-1.11 (m, 3H), 0.64 (s, 2H), 0.43 (s, 2H) |
| 110 | 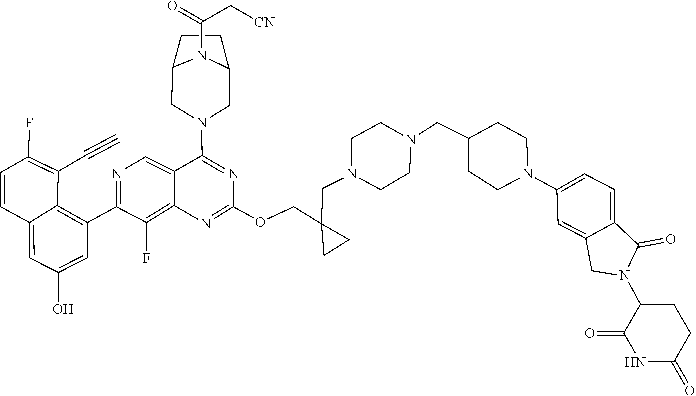 [SMILES: C#Cc1c(F)ccc2cc(O)cc(-c3ncc4c(N5CC6CCC(C5)N6C(=O)CC#N)nc(OCC5(CN6CCN(CC7CCN(c8ccc9c(c8)CN(C8CCC(=O)NC8=O)C9=O)CC7)CC6)CC5)nc4c3F)c12] | 1018.7 | δ 10.94 (s, 1H), 9.02 (d, J = 3.8 Hz, 1H), 8.21 (s, 1H), 7.98 (dd, J = 5.8, 9.4 Hz, 1H), 7.52-7.43 (m, 2H), 7.40 (d, J = 2.4 Hz, 1H), 7.17 (s, 1H), 7.06-6.98 (m, 2H), 5.03 (dd, J = 4.8, 13.4 Hz, 1H), 4.72-4.65 (m, 1H), 4.65-4.54 (m, 1H), 4.44-4.35 (m, 2H), 4.35-4.26 (m, 4H), 4.22-4.19 (m, 1H), 4.17-4.12 (m, 2H), 3.95 (d, J = 7.6 Hz, 1H), 3.88-3.78 (m, 4H), 2.94-2.85 (m, 3H), 2.83-2.75 (m, 4H), 2.34-2.28 (m, 6H), 2.09 (d, J = 6.6 Hz, 2H), 1.98-1.92 (m, 2H), 1.86-1.70 (m, 6H), 1.24-1.06 (m, 3H), 0.64 (s, 2H), 0.41 (s, 2H). |
| 111 | 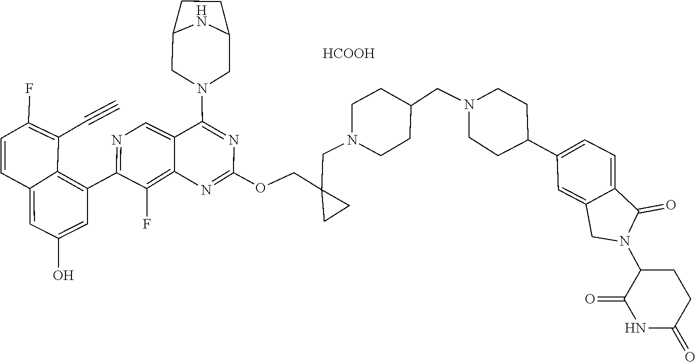 [SMILES: C#Cc1c(F)ccc2cc(O)cc(-c3ncc4c(N5CC6CCC(C5)N6)nc(OCC5(CN6CCC(CN7CCC(c8ccc9c(c8)CN(C8CCC(=O)NC8=O)C9=O)CC7)CC6)CC5)nc4c3F)c12 ; "HCOOH"] | 950.4 | 0.99 (s, 1H), 9.04 (s, 1H), 8.19 (s, 1H), 7.98 (dd, J = 5.8, 9.0 Hz, 1H), 7.64 (d, J = 7.8 Hz, 1H), 7.50-7.44 (m, 2H), 7.41- 7.37 (m, 2H), 7.18 (d, J = 2.6 Hz, 1H), 5.10 (dd, J = 5.2, 13.2 Hz, 1H), 4.51 (br d, J = 12.0 Hz, 1H), 4.42 (d, J = 17.4 Hz, 1H), 4.34-4.25 (m, 4H), 3.95 (s, 1H), 3.68 (s, 1H), 3.64 (d, J = 6.0 Hz, 3H), 3.60 (s, 1H), 3.57 (s, 1H), 2.98- 2.87 (m, 6H), 2.63-2.57 (m, 2H), 2.34- 2.31 (m, 2H), 2.12 (d, J = 6.8 Hz, 2H), 2.01-1.94 (m, 3H), 1.91-1.84 (m, 2H), 1.75-1.65 (m, 9H), 1.52-1.43 (m, 1H), 1.12-1.01 (m, 2H), 0.69-0.59 (m, 2H), 0.46-0.37 (m, 2H) |
| 112 | 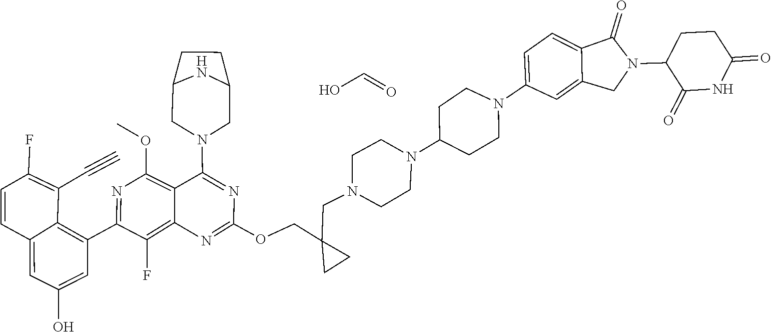 [SMILES: C#Cc1c(F)ccc2cc(O)cc(-c3nc(OC)c4c(N5CC6CCC(C5)N6)nc(OCC5(CN6CCN(C7CCN(c8ccc9c(c8)CN(C8CCC(=O)NC8=O)C9=O)CC7)CC6)CC5)nc4c3F)c12 ; O=CO] | 967.6 | 8.25 (s, 1H), 7.97 (dd, J = 6.0, 9.2 Hz, 1H), 7.53-7.42 (m, 2H), 7.40 (d, J = 2.6 Hz, 1H), 7.23 (d, J = 2.6 Hz, 1H), 7.06- 6.95 (m, 2H), 5.01 (dd, J = 5.2, 13.4 Hz, 1H), 4.36-4.23 (m, 3H), 4.22-4.10 (m, 2H), 4.00-3.89 (m, 2H), 3.87 (d, J = 3.6 Hz, 3H), 3.85-3.79 (m, 2H), 3.74 (s, 2H), 3.49-3.38 (m, 4H), 2.94-2.83 (m, 1H), 2.82-2.70 (m, 2H), 2.64-2.54 (m, 2H), 2.46-2.32 (m, 8H), 2.27 (s, 2H), 2.02-1.90 (m, 1H), 1.85-1.61 (m, 6H), 1.49-1.33 (m, 2H), 0.61 (s, 2H), 0.40 (s, 2H) |
| 113 | 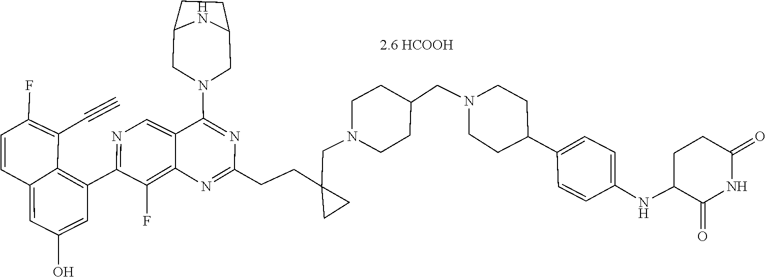 | 910.4 | 10.77 (s, 1H), 9.04 (s, 1H), 8.20 (s, 3H), 7.97 (d, J = 6.2 Hz, 1H), 7.43 (s, 1H), 7.37 (s, 1H), 7.18 (d, J = 2.6 Hz, 1H), 6.94 (d, J = 8.4 Hz, 2H), 6.60 (d, J = 8.4 Hz, 2H), 5.64 (d, J = 7.4 Hz, 1H), 4.51 (d, J = 12.8 Hz, 1H), 4.34-4.24 (m, 4H), 3.94 (s, 1H), 3.62 (s, 3H), 2.98- 2.92 (m, 3H), 2.88 (d, J = 10.4 Hz, 3H), 2.79-2.70 (m, 2H), 2.61-2.58 (m, 1H), 2.35-2.30 (m, 3H), 2.13-2.07 (m, 3H), 1.94-1.83 (m, 5H), 1.73-1.60 (m, 9H), 1.13-0.98 (m, 2H), 0.64 (s, 2H), 0.41 (s, 2H) |
| 114 | 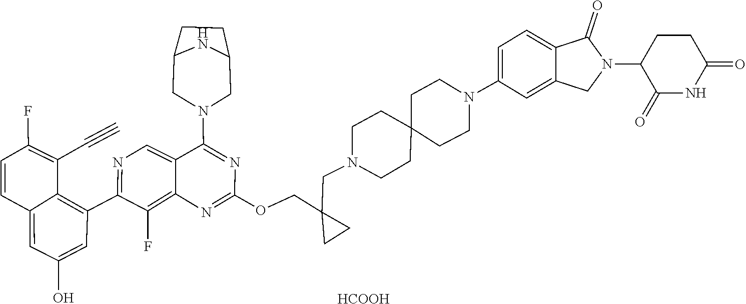 [SMILES: C#Cc1c(F)ccc2cc(O)cc(-c3ncc4c(N5CC6CCC(C5)N6)nc(OCC5(CN6CCC7(CC6)CCN(c6ccc8c(c6)CN(C6CCC(=O)NC6=O)C8=O)CC7)CC5)nc4c3F)c12 ; "HCOOH"] | 922.6 | 11.01-10.87 (m, 1H), 9.04 (s, 1H), 8.19 (s, 2H), 7.97 (dd, J = 6.0, 9.4 Hz, 1H), 7.52-7.44 (m, 2H), 7.39 (d, J = 2.4 Hz, 1H), 7.17 (d, J = 2.4 Hz, 1H), 7.07-6.94 (m, 2H), 5.03 (dd, J = 5.0, 12.9 Hz, 1H), 4.51 (d, J = 12.0 Hz, 1H), 4.33- 4.16 (m, 5H), 3.94 (s, 1H), 3.63 (s, 2H), 3.28 (s, 3H), 2.61-2.54 (m, 2H), 2.44- 2.33 (m, 9H), 1.99-1.91 (m, 1H), 1.70 (s, 4H), 1.46 (d, J = 14.0 Hz, 8H), 0.64 (s, 2H), 0.42 (s, 2H) |
| 115 | 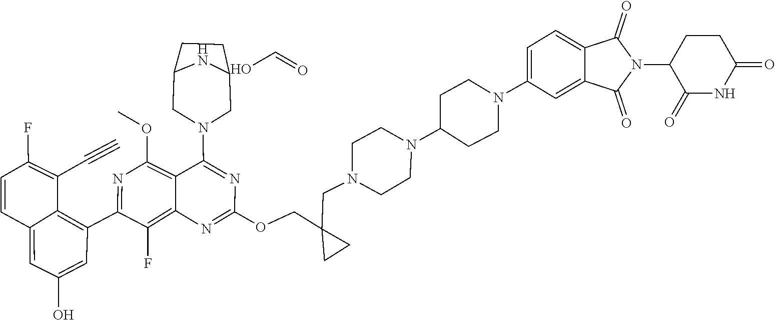 | 981.5 | 11.07 (s, 1H), 8.20 (s, 1H), 7.97 (dd, J = 5.8, 9.4 Hz, 1H), 7.64 (d, J = 8.6 Hz, 1H), 7.46 (t, J = 9.0 Hz, 1H), 7.38 (d, J = 2.6 Hz, 1H), 7.29 (s, 1H), 7.23-7.18 (m, 2H), 5.06 (dd, J = 5.4, 12.4 Hz, 1H), 4.27 (s, 2H), 4.16-4.09 (m, 1H), 4.03-3.98 (m, 2H), 3.89 (s, 1H), 3.88 (s, 3H), 3.56 (s, 6H), 2.98-2.87 (m, 4H), 2.69-2.57 (m, 2H), 2.46-2.35 (m, 8H), 2.27 (s, 2H), 2.03-1.97 (m, 1H), 1.79 (dd, J = 2.2, 13.0 Hz, 2H), 1.71-1.58 (m, 4H), 1.46-1.36 (m, 2H), 0.61 (s, 2H), 0.39 (s, 2H) |
| 116 | 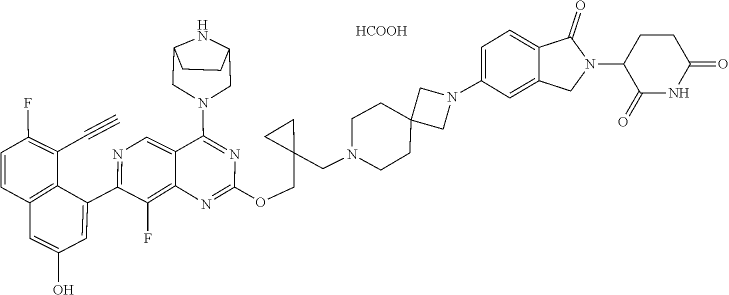 [SMILES: C#Cc1c(F)ccc2cc(O)cc(-c3ncc4c(N5CC6CCC(C5)N6)nc(OCC5(CN6CCC7(CC6)CN(c6ccc8c(c6)CN(C6CCC(=O)NC6=O)C8=O)C7)CC5)nc4c3F)c12 ; "HCOOH"] | 894.5 | 10.94 (s, 1H), 10.16 (s, 1H), 9.05 (s, 1H), 8.17 (s, 1H), 7.98 (dd, J = 6.2, 9.4 Hz, 1H), 7.50-7.44 (m, 2H), 7.40 (d, J = 2.6 Hz, 1H), 7.18 (d, J = 2.4 Hz, 1H), 6.53-6.41 (m, 2H), 5.03 (dd, J = 4.6, 13.0 Hz, 1H), 4.56-4.48 (m, 1H), 4.35- 4.25 (m, 4H), 4.20-4.13 (m, 1H), 3.95 (s, 1H), 3.70-3.56 (m, 9H), 2.95-2.86 (m, 2H), 2.63-2.57 (m, 2H), 2.30 (d, J = 8.6 Hz, 4H), 2.00-1.89 (m, 2H), 1.71 (s, 8H), 0.69-0.63 (m, 2H), 0.46-0.40 (m, 2H) |
| 117 | 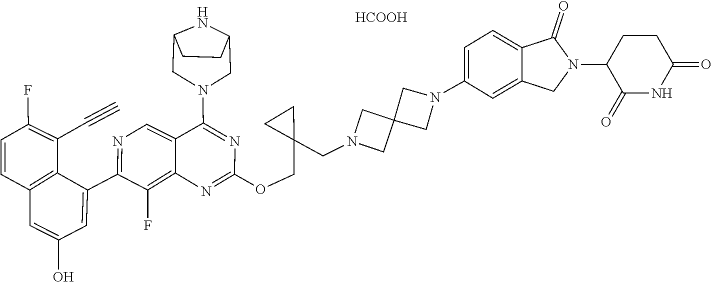 [SMILES: C#Cc1c(F)ccc2cc(O)cc(-c3ncc4c(N5CC6CCC(C5)N6)nc(OCC5(CN6CC7(C6)CN(c6ccc8c(c6)CN(C6CCC(=O)NC6=O)C8=O)C7)CC5)nc4c3F)c12 ; "HCOOH"] | 866.4 | 10.94 (s, 1H), 9.05 (s, 1H), 8.21 (s, 1H), 7.98 (dd, J = 6.0, 9.2 Hz, 1H), 7.50- 7.44 (m, 2H), 7.40 (d, J = 2.4 Hz, 1H), 7.19 (d, J = 2.4 Hz, 1H), 6.51-6.42 (m, 2H), 5.06-4.99 (m, 1H), 4.53-4.45 (m, 1H), 4.34-4.13 (m, 6H), 3.97-3.95 (m, 4H), 3.68-3.63 (m, 2H), 3.61-3.55 (m, 5H), 2.96-2.84 (m, 2H), 2.60 (dd, J = 1.8, 2.8 Hz, 1H), 2.56 (s, 1H), 2.41 (s, 2H), 2.36 (d, J = 4.2 Hz, 1H), 2.01-1.90 (m, 1H), 1.69 (s, 3H), 1.27-1.21 (m, 1H), 0.54 (s, 2H), 0.45 (s, 2H) |
| 118 | 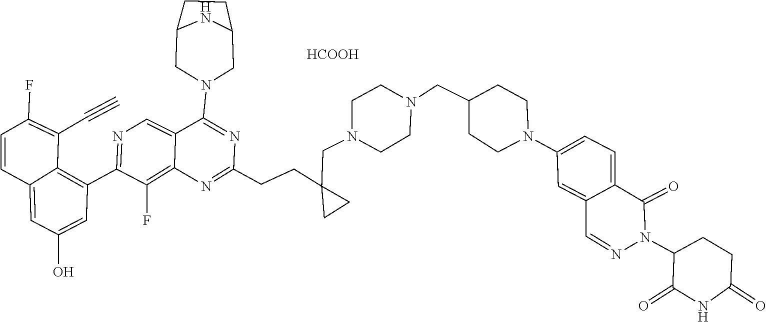 | 965.0 | 10.99 (s, 1H), 10.26-10.07 (m, 1H), 9.03 (s, 1H), 8.26 (s, 1H), 8.23 (s, 1H), 8.04-7.94 (m, 2H), 7.51-7.42 (m, 2H), 7.39 (d, J = 2.6 Hz, 1H), 7.22 (d, J = 1.8 Hz, 1H), 7.18 (d, J = 2.4 Hz, 1H), 5.74 (dd, J = 5.4, 11.0 Hz, 1H), 4.50 (dd, J = 1.8, 12.4 Hz, 1H), 4.32-4.24 (m, 3H), 4.03-3.96 (m, 2H), 3.94 (s, 1H), 3.68-3.62 (m, 1H), 3.60-3.51 (m, 5H), 2.97-2.86 (m, 4H), 2.65-2.57 (m, 3H), 2.30 (d, J = 6.0 Hz, 5H), 2.14-2.03 (m, 4H), 1.82-1.73 (m, 3H), 1.67 (s, 4H), 1.27-1.07 (m, 3H), 0.64 (s, 2H), 0.41 (s, 2H). |

### Biological Assays

### Materials and Methods

### Cell Lines

The following cancer cell lines were employed: AsPC-1 human pancreatic adenocarcinoma (ATCC, #CRL-1682); A-427 human lung carcinoma (ATCC, #HTB-53); BA/F3-KRAS(G12D) murine pro-B cell line stably expressing KRAS(G12D) (Precedo, Hefei, China). Cell lines were cultured according to ATCC and recommendations. BA/F3-KRAS(G12D) cells were cultured in RPMI 1640 media with 10% fetal calf serum, 100 units/mL penicillin and 100 mg/mL streptomycin, and 1% glutamine.

### KRAS(G12D)/SOS1 Homogeneous Time-Resolved Fluorescence (HTRF) Assay

Binding of test compounds to KRAS(G12D) protein, which in turn blocks KRAS(G12D) interaction with the SOS1 protein, was measured in the absences of GTP by homogeneous time-resolved fluorescence (HTRF) using the KRAS-(G12D)/SOS1 Binding Assay Kit (Cisbio, #63ADK000CB16PEG), following the manufacturer's instructions, except as noted. 3-fold serial dilutions of each test compound were prepared ranging from 20 μM to 1.02 nM. The test compound was mixed and incubated with reaction components, incubated in a sealed plate at 4° C. for 3 hr and fluorescence was measured using a PerkinElmer Envision plate reader. The % inhibition and IC50 values (the concentration at which 50% of the maximal inhibition occurs) were calculated and plotted using GraphPad Prism 7 software.

### CellTiter-Glo® Reagent Cancer Cell Line Proliferation Assays

Cells were plated in 96-well tissue culture plates at 4,000 cells/well and incubated at 37° C./5% CO₂ for 24 hr in 100 μl of media. 3-fold serial dilutions of each test compound were prepared ranging from 20 μM to 1.02 nM. Cells were then treated with test compounds at various concentrations with a final concentration of 0.5% DMSO/well, and then incubated at 37° C./5% CO₂ for 24 hr. 100 μl of CellTiter-Glo® Reagent (Promega Corporation, Madison, WI) was added to each well and processed according manufacturer's protocol. Results were analyzed and IC50-values were calculated in GraphPad 7 software.

### Results

A number of synthetic schemes have been developed to construct various PROTAC molecules designed to degrade KRAS(G12D), which are termed KRAS(G12D)-PROTAC molecules. Representative examples are shown, each consisting of a E3 ubiquitin-ligase binder (CRBN or VHL) linked to a KRAS(G12D) binder. Similar chemistry can be applied to other PROTAC molecules not limited to these specific E3 ubiquitin-ligase- and KRAS(G12D)-binding moieties.

Binding by a variety of PROTAC molecules to KRAS(G12D) was assessed by measuring inhibition of KRAS(G12D) interaction with SOS1 in a HTRF biochemical assay, as shown in Table 1. PROTAC molecules containing KRAS(G12D)-binding moieties documented in the literature were generally in agreement with the published SAR.

KRAS(G12D)-PROTAC molecules also inhibited the growth and/or survival of a panel of cancer cell lines as measured by CellTiter-Glo® Reagent cancer cell line proliferation assay as shown in Table 1.

TABLE 1

| | KRAS | | | | | | | |
|---|---|---|---|---|---|---|---|---|
| | (G12D)/ | ASPC1 at | A427 at | BAF3 at | | | | ³WB data |
| | SOS1, | 24 hrs, | 24 hrs, | 24 hrs, | | | | SW1990 |
| | IC50 | IC50 | IC50 | IC50 | | | | DC₅₀; |
| Cmp. | (nM)¹ | (nM)² | (nM)² | (nM)² | AGS | SW1990 | GP2D | D_{Max} |
| 1 | B | | C | C | | | | |
| 2 | A | C | C | C | | | | |
| 3 | A | | C | B | | | | |
| 4 | C | | C | C | | | | |
| 5 | A | C | C | B | | | | |
| 6 | A | C | C | C | | | | |
| 7 | A | C | C | C | | | | |
| 8 | A | C | C | C | | | | |
| 9 | A | C | C | C | | | | |
| 10 | A | C | C | C | | | | |
| 11 | B | C | C | C | | | | |
| 12 | B | C | C | C | | | | |
| 13 | A | C | C | B | | | | |
| 14 | A | B | C | B | | | | |
| 15 | A | | C | C | | | | |
| 16 | A | | C | C | | | | |
| 17 | A | C | C | C | | | | |
| 18 | A | C | C | C | | | | |
| 19 | B | C | C | C | | | | |
| 20 | C | | | C | | | | |
| 21 | B | C | C | C | | | | |
| 22 | C | C | C | C | | | | |
| 23 | C | C | C | C | | | | |
| 24 | B | C | C | C | | | | |
| 25 | A | C | C | C | | | | |
| 26 | A | C | C | C | | | | |
| 27 | B | C | C | C | | | | |
| 28 | A | C | C | C | | | | |
| 29 | A | C | C | B | | | | |
| 30 | A | C | C | C | | | | |
| 31 | A | C | C | C | | | | |
| 32 | B | C | C | | C | | | |
| 33 | A | C | C | | C | | | |
| 34 | A | C | C | | C | | | |
| 35 | A | C | C | | C | | | |
| 36 | A | C | C | | C | | | |
| 37 | A | C | C | | C | | | |
| 38 | A | C | C | | C | | | |
| 39 | A | C | C | | B | | | |
| 40 | A | C | C | | B | | | |
| 41 | A | C | C | | C | | | |
| 42 | A | C | C | | B | | | |
| 43 | A | C | C | | B | | | |
| 44 | A | C | C | | C | C | | |
| 45 | A | CC | C | | C | C | | |
| 46 | A | C | C | | C | C | | |
| 47 | A | C | C | | B | | | |
| 48 | A | C | C | | C | | | |
| 49 | B | — | — | | — | | | |
| 50 | A | C | C | | C | | | |
| 51 | A | C | C | | C | | | |
| 52 | A | — | B | | A | | | |
| 53 | A | — | C | | C | | | |
| 54 | A | — | C | | C | | | |
| 55 | A | — | C | | C | | | |
| 56 | A | — | B | | A | A | | A; A |
| 57 | A | A | A | | A | A | A | A; A |
| 58 | A | — | — | | C | | | |
| 59 | A | B | B | | A | A | | |
| 60 | A | — | C | | C | | | |
| 61 | A | — | B | | B | | | |
| 62 | A | — | B | | C | | | |
| 63 | A | — | C | | C | | | |
| 64 | A | — | C | | B | | | |
| 65 | A | — | C | | B | | | |
| 66 | A | B | B | | B | B | | |
| 67 | A | — | B | | C | | | |
| 68 | A | — | C | | B | | | |
| 69 | A | — | C | | A | | | |
| 70 | C | — | C | | C | | | |
| 71 | A | — | B | | A | A | | C; B % |
| 72 | A | — | B | | A | B | | C; B % |
| 73 | A | A | A | | A | A | A | A; A |
| 74 | A | — | C | | C | | | |
| 75 | A | — | C | | B | | | |
| 76 | A | — | C | | C | | | |
| 77 | A | C | CC | | C | C | | |
| 78 | A | A | A | | A | A | A | A; A % |
| 79 | A | C | C | | C | C | | |
| 80 | A | C | C | | C | C | | |
| 81 | A | B | B | | A | A | | C; B |
| 82 | A | C | C | | B | C | | A; A |
| 83 | A | C | C | | B | C | | C; B |
| 84 | — | C | C | | A | B | | B; A |
| 85 | — | C | A | | A | B | | B; A % |
| 86 | — | A | A | | A | A | | A; A |
| 87 | — | B | B | | A | B | | C; B |
| 88 | — | B | C | | A | B | | |
| 89 | — | C | C | | A | B | | |
| 90 | — | A | A | | A | A | | |
| 91 | — | C | C | | C | C | | C; C |
| 92 | A | C | C | | C | C | | |
| 93 | A | B | C | | A | B | A | B; B |
| 94 | A | A | B | | A | A | A | A; A |
| 95 | A | B | B | | A | A | A | C; C |
| 96 | A | A | B | | A | A | A | C; B |
| 97 | A | C | C | | C | C | C | |
| 98 | C | C | C | | C | C | C | C; C |
| 99 | A | A | C | | A | A | A | A; A |
| 100 | A | A | B | | A | A | A | C; B |
| 101 | — | B | C | | B | B | A | C; C |
| 102 | A | A | B | | A | A | A | B; B |
| 103 | A | B | C | | A | A | A | C; B |
| 104 | A | B | — | | A | A | A | A; A |
| 105 | A | C | — | | C | C | B | |
| 106 | A | C | — | | C | C | C | |
| 107 | A | A | — | | A | A | A | A; A |
| 108 | A | — | — | | A | B | A | |
| 109 | A | — | — | | B | C | B | |
| 110 | A | — | — | | C | C | B | |
| 111 | A | — | — | | A | A | A | A; A |
| 112 | A | — | — | | A | A | A | A; A |
| 113 | A | — | — | | A | A | A | |
| 114 | A | — | — | | A | A | A | |
| 115 | A | — | — | | A | A | A | |
| 116 | A | — | — | | A | A | A | |
| 117 | A | — | — | | A | C | A | |
| 118 | — | — | — | | A | A | A | |

¹KRAS(G12D)/SOS1 homogeneous time-resolved fluorescence (HTRF) assay: A. IC50 < 100 nM; B. IC50 = 100-1000 nM; C. IC50 > 1000 nM;
²CellTiter-Glo ® Reagent cancer cell line proliferation assays: A. IC50 < 100 nM; B. IC50 = 100-1000 nM; C. IC50 > 1000 nM;
³SW1990 WB assay: A. DC50 < 100 nM; B. DC50 = 100-1000 nM; C. DC50 > 1000 nM; A. Dmax > 50%; B. Dmax = 10-50%; C. Dmax < 10%.

One of the disclosed compounds was advanced to a KRAS(G12D) degradation assay as measured by Western blot in A-427 cells and demonstrated considerable efficacy. Furthermore, the same compound caused significantly more cytotoxicity than the KRAS(G12D) binding moiety or the E3-ligase binding moiety measured alone or in combination in BA/F3-KRAS(G12D) cells.

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A compound of the Formula I: 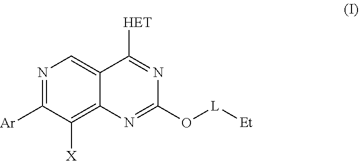
or a pharmaceutically acceptable salt thereof, wherein
HET is an optionally substituted heterocyclyl;
Ar is an optionally substituted aryl or optionally substituted heteroaryl;
X is hydrogen or halo;
L is a linker; and
E is a chemical moiety that targets E3 ligase.

2. The compound of claim 1, wherein the compound is of the Formula II: 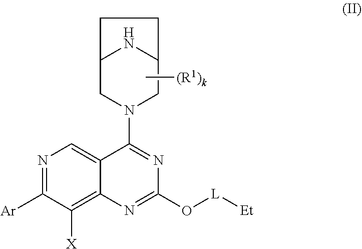
or a pharmaceutically acceptable salt thereof, wherein
R¹ is halo, (C₁-C₄)alkyl, hydroxy(C₁-C₄)alkyl, cyano(C₁-C₄)alkyl, —CH(═O), —C(O)₂H, —C(O)₂(C₁-C₄)alkyl, C(O)₂NH₂, —C(O)₂NH(C₁-C₄)alkyl, —C(O)₂N[(C₁-C₄)alkyl]₂, or a 5- to 6-membered optionally substituted heteroaryl; and
k is 0, 1, 2, or 3.

3. The compound of claim 1 or 2, wherein the compound is of the Formula III: 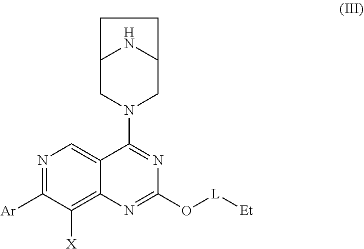
or a pharmaceutically acceptable salt thereof.

4. The compound of any one of claims 1 to 3, or a pharmaceutically acceptable salt thereof, wherein X is hydrogen or fluoro.

5. The compound of any one of claims 1 to 4, or a pharmaceutically acceptable salt thereof, wherein X is fluoro.

6. The compound of any one of claims 1 to 5, or a pharmaceutically acceptable salt thereof, wherein Ar is an optionally substituted phenyl or optionally substituted naphthyl.

7. The compound of any one of claims 1 to 6, or a pharmaceutically acceptable salt thereof, wherein Ar is phenyl or naphthalenyl, each of which are optionally substituted with one to three groups independently selected from R^{A}, wherein R^{A} is selected from halo, (C₁-C₄)alkyl, (C₂-C₄)alkynyl, (C₂-C₄)alkynylNH₂, (C₁-C₄)alkoxy, halo(C₁-C₄)alkoxy, (C₁-C₄)alkylOH, OH, NH₂, —NH(C₁-C₄)alkyl, —N[(C₁-C₄)alkyl]₂, C(O)NH₂, C(O)NH(C₁-C₄)alkyl, C(O)[(C₁-C₄)alkyl]₂, —NHC(O)(C₁-C₄)alkyl, —N(C₁-C₄)alkylC(O)(C₁-C₄)alkyl, —NHC(O)O(C₁-C₄)alkyl, NHC(O)NH(C₁-C₄)alkyl, CN, —S(C₁-C₄)alkyl, —Shalo(C₁-C₄)alkyl, and (C₃-C₆)cycloalkyl, wherein said (C₁-C₄)alkyl and said (C₃-C₆)cycloalkyl are each optionally substituted with one to two groups selected from halo, (C₁-C₄)alkoxy, halo(C₁-C₄)alkoxy, OH, NH₂, —NH(C₁-C₄)alkyl, —N[(C₁-C₄)alkyl]₂, C(O)NH₂, C(O)NH(C₁-C₄)alkyl, C(O)[(C₁-C₄)alkyl]₂, —NHC(O)(C₁-C₄)alkyl, —N(C₁-C₄)alkylC(O)(C₁-C₄)alkyl, —NHC(O)O(C₁-C₄)alkyl, NHC(O)NH(C₁-C₄)alkyl, CN, —S(C₁-C₄)alkyl, and —S(C₁-C₄) haloalkyl.

8. The compound of any one of claims 1 to 7, or a pharmaceutically acceptable salt thereof, wherein Ar is naphthalenyl optionally substituted with one to three groups independently selected from R^{A}.

9. The compound of claim 7 or 8, or a pharmaceutically acceptable salt thereof, wherein R^{A} is selected from (C₂-C₄)alkynyl, halo, and OH.

10. The compound of any one of claims 1 to 9, or a
or a pharmaceutically acceptable salt thereof, wherein AR is

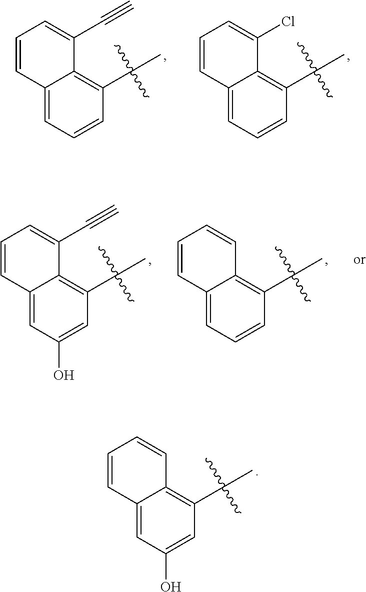 [SMILES: *c1cccc2cccc(C#C)c12 ; *c1cccc2cccc(Cl)c12 ; *c1cc(O)cc2cccc(C#C)c12 ; *c1cccc2ccccc12 ; *c1cc(O)cc2ccccc12]

11. The compound of any one of claims 1 to 10, or a pharmaceutically acceptable salt thereof, wherein E is selected from a cereblon (CRBN) modulator and a von Hippel-Lindau (VHL) ligand.

12. The compound of any one of claims 1 to 11, or a pharmaceutically acceptable salt thereof, wherein E is of the structural formula: 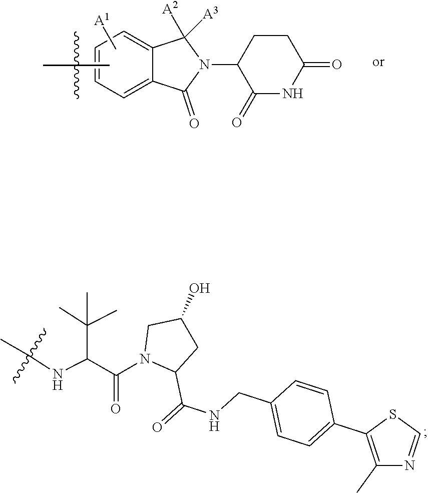
wherein A¹ is halo; and A² and A³ are both hydrogen or A² and A³ taken together form ═O.

13. The compound of any one of claims 1 to 12, or a pharmaceutically acceptable salt thereof, wherein E is of the structural formula:

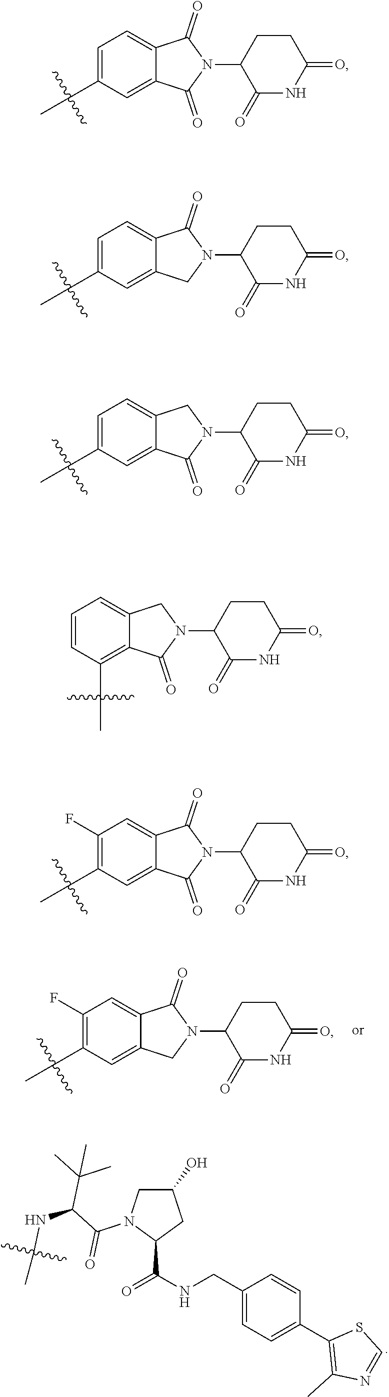

14. The compound of any one of claims 1 to 13, or a pharmaceutically acceptable salt thereof, wherein
L is -Het¹-X¹—*, *-Het¹-Het²-X¹—, *-NR^{c}—X¹-Het¹-Het²-X²—,*-X¹-Het¹-X²-Het²-(CH₂)ₘO—, —NR^{c}—(CH₂)ₘ—X¹-Het¹-X²—*, —NR^{c}—(CH₂)ₘ—X¹—NR^{c}—(CH₂CH₂O)ₙ—*, —NR^{c}—(CH₂)ₘ—X¹—NR^{c}—(CH₂)ₚ—*, —NR^{c}—(CH₂)ₘ—X¹-Het¹-X²-Het²-X³-*, —O(CH₂)ₘ—X¹-Het¹-X²-Het²-X³-*, —O(CH₂)ₘ—X¹—NR^{c}—(CH₂)ₚ-Het¹-X²-Het²-X³-*, *-X¹—NR^{c}—(CH₂)ₘ Het¹-X²-Het²-X³—(CH₂)ₚ—NR^{d}—(CH₂)ₚ—, —NR^{c}—(CH₂)ₘ—X¹—(CH)CH₃—Het¹-X²-Het³-X³-*, —NR^{c}—(CH₂)ₘ—X¹—(CH₂)ₚ-Het¹-X²-Het²-X³-*, —NR^{c}—(CH₂)ₘ—X¹—NR^{d}—(CH₂)ₚ-Het¹-X²-Het²-X³-*, —NR^{c}—(CH₂)ₘ—NR^{d}—X¹-Het¹-X²—*, *Het¹-X¹-Het²-X²—, *X¹-Het-X²—, *(CH₂CH₂O)ₙ—NR^{c}—X¹-Het¹-Het²-X²—, *Het¹-X¹-Het²-X²-Het³-X³—, *X¹-Het-X²-Het²-X³—, *-Het¹-X¹—NR^{c}-Het²-X², *X¹-Het¹-Het²-X²—, *X¹—(CH₂)ₘO-Het¹-X²—, *X¹—(CH₂)ₘNR^{c}—X²-Het¹-Het²-X²—, *-Het¹-X¹-Het²-X²—O—, *-O(CH₂)ₘ-Het¹-(CH₂)ₚ—O(CH₂)ₘ—NR^{c}—X²—*, *-Het¹-O—(CH₂)ₘ—X¹-Het²-X²—, *-Het¹-O—(CH₂)ₘ—X¹—NR^{c}—(CH₂CH₂O)ₙ(CH₂)ₘ-Het²-X²—, *-Het¹-X¹—NR^{c}—(CH₂)ₘ—, *-Het¹-X¹-Het²-Het³-X²—, *-Het¹-X¹—NR^{c}—(CH₂CH₂O)ₙ(CH₂)ₘ—, *-Het¹-X¹—NR^{c}—(CH₂CH₂O)ₙHet²-(CH₂)ₘ—X²—, *-Het¹-X¹—NR^{c}—(CH₂CH₂O)ₙ—, *-Het¹-X¹—NR^{c}—(CH₂)ₘ-Het²-X²-Het³-(CH₂)ₘ—, *-Het¹-X¹-Het²-(CH₂)ₘ-Het³-X²—, *-Het¹-X¹-Het²-, *-Het¹-X¹—NR^{c}—, *-Het¹-X¹—NR^{c}—(CH₂)ₘ-Phe-X²-Het²-(CH₂)ₘ—, *-Het¹-X¹-Het²-Het³-, *-Het¹-X¹-Het²-(CH₂)ₘ-Het³-X²—(CH₂)ₚ—NR^{c}—(CH₂)ₘ—, *-Het¹-X¹-Het²-(CH₂)ₘ-Het³-(CH₂)ₘ—O—, *-Het¹-X¹-Het²-(CH₂)ₘ Het³-(CH₂)ₚ—NR^{c}—(CH₂)ₘ—, *-Het¹-X¹-Het²-(CH₂CH₂O)ₙ—,*-Het¹-X¹—(CH₂)ₘ-Het²-X²—, *- (CH₂CH₂O)ₒ—(CH₂)ₚ-Het¹-X¹-Het²-(CH₂CH₂O)ₙ, *-(CH₂CH₂O)ₙ—(CH₂)ₘ-Het¹-X¹-Het²-X², *-Het¹-X¹-Phe-X²—NRK—X³—, *-(CH₂CH₂O)ₒ—(CH₂)ₚ-Het¹-X¹-Phe-X²—NR^{c}—(CH₂CH₂O)ₙ—, *-(CH₂CH₂O)ₙ—(CH₂)ₘ—NR^{c}-Phe-X¹—, *-(CH₂CH₂O)ₒ—(CH₂)ₚ—NR^{c}-Phe-(CH₂CH₂O)ₙ—, *-(CH₂CH₂O)ₒ—(CH₂)ₚ—NR^{c}—(CH₂CH₂O)ₙ—(CH₂)ₘ—, *-(CH₂CH₂O)ₙ—(CH₂)ₘ—NR^{c}—(CH₂CH₂O)ₙ—(CH₂)ₘ—C(O)—NR^{d}—(CH₂CH₂O)ₒ—(CH₂)ₚ—, *-(CH₂CH₂O)ₒ—(CH₂)ₚ—NR^{c}—(CH₂CH₂O)ₙ—(CH₂)ₘ-Het¹-X¹-Het²-X²—, *-(CH₂CH₂O)ₒ—(CH₂)ₚ—NR^{c}—(CH₂CH₂O)ₙ—(CH₂)ₘ Het¹-X¹-Het²-X²—(CH₂CH₂O)ₒ, *-NR^{c}—(CH₂CH₂O)ₙ—(CH₂)ₘ-Phe-NH—X¹-Het¹-X², *-NR^{c}—(CH₂CH₂O)ₙ—(CH₂)ₘ-Phe-NH—X¹-Het¹-X²—(CH₂CH₂O)ₒ, *-(CH₂CH₂O)ₒ—(CH₂)ₚ—NR^{c}—(CH₂)ₘ-Phe-X¹—NR^{c}—(CH₂CH₂O)ₒ—(CH₂)ₚ—, *-(CH₂CH₂O)ₒ—(CH₂)ₚ—NR^{c}—(CH₂CH₂O)ₙ—(CH₂)ₘ-Het¹-X¹—, *-(CH₂CH₂O)ₒ—(CH₂)ₚ—NR^{c}—(CH₂CH₂O)ₙ—(CH₂)ₘ-Het¹- X¹—(CH₂CH₂O)ₙ—, *-(CH₂CH₂O)ₙ—(CH₂)ₘ—NR^{c}—(CH₂)ₘ—C(O)—NR^{d}-Het¹-X¹-Het²-(CH₂CH₂O)ₒ—(CH₂)ₚ, or *-NR^{c}—(CH₂)ₘ—C(O)—NR^{d}—(CH₂)ₘ-Het¹-X¹-Het²-X²_;
* indicates the point of attachment to E;
Het¹, Het², and Het³ are each independently phenyl, a 5- to 8-membered heterocyclyl, 5- to 7-membered heteroaryl, or a 3- to 6-membered cycloalkyl, each of which are optionally substituted with (C₁-C₄)alkyl;
Phe is phenyl;
X¹, X², and X³, are each independently C(O) or (CH₂)ᵣ;
R^{c} and R^{d} are each independently hydrogen or (C₁-C₄)alkyl; and
m, n, o, p, and r are each independently integers selected from 0, 1, 2, 3, 4, 5, and 6.

15. The compound of any one of claims 1 to 14, or a pharmaceutically acceptable salt thereof, wherein L is *-Het¹-Het²-X¹—, *-NR^{c}—X¹-Het¹-Het²-X²—, *Het¹-X¹-Het²-X²—, *X¹-Het¹-X²—, *(CH₂CH₂O)ₙ—NR^{c}—X¹-Het¹-Het²-X²—, *Het¹-X¹-Het²-X²-Het³-X³—, *X¹-Het¹-X²-Het²-X³—, *-Het¹-X¹—NR^{c}-Het²-X²—, *X¹-Het¹-Het²-X²—, *X¹—(CH₂)ₘO-Het-X²—, *X¹—(CH₂)ₘNR^{c}—X²-Het¹-Het²-X²—, or *-Het¹-X¹-Het²-Het³-X²—.

16. The compound of claim 14 or 15, or a pharmaceutically acceptable salt thereof, wherein Het¹, Het², and Het³ are each independently a 5- to 8-membered heterocyclyl, or a 3- to 6-membered cycloalkyl, each of which are optionally substituted with (C₁-C₄)alkyl.

17. The compound of any one of claims 14 to 16, or a pharmaceutically acceptable salt thereof, wherein m, n, o, p, and r are each independently integers selected from 0, 1, 2, or 3.

18. The compound of any one of claims 1 to 17, or a pharmaceutically acceptable salt thereof, wherein L is selected from

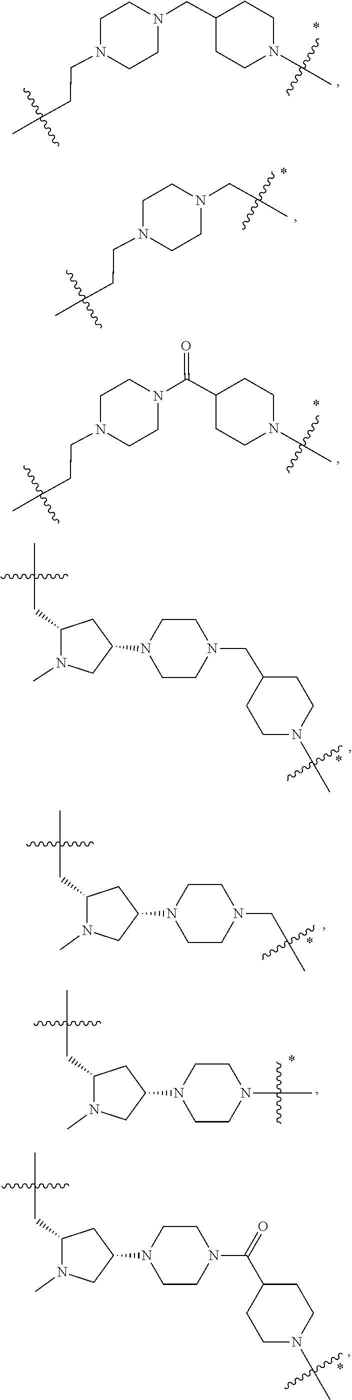

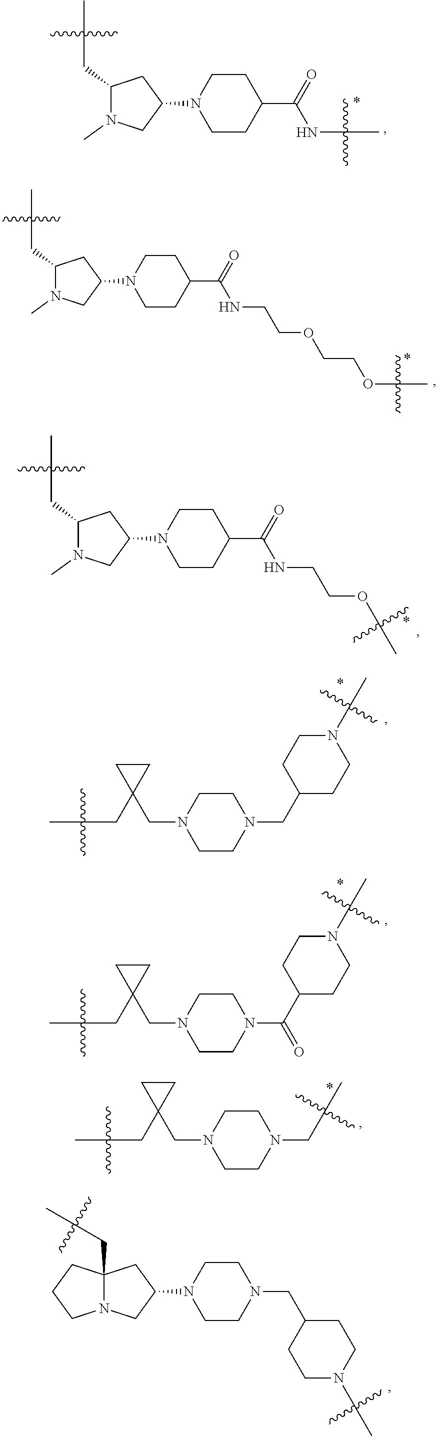

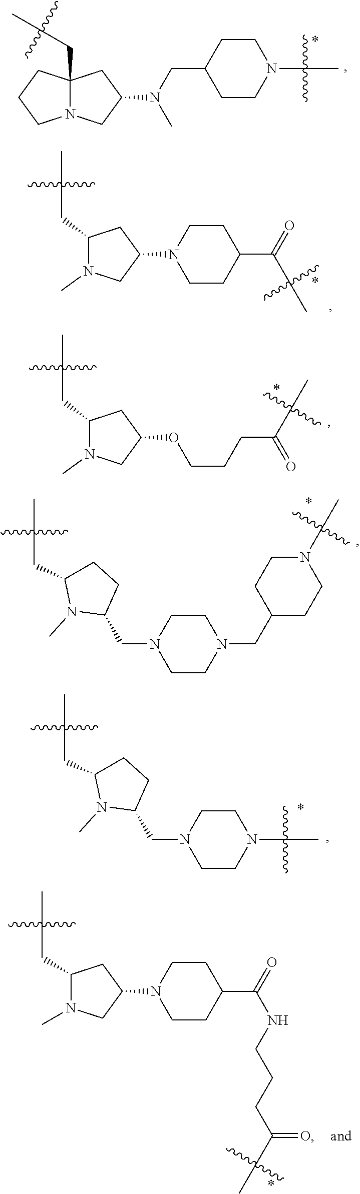 [SMILES: *C[C@]12CCCN1C[C@@H](N(C)CC1CCN(*)CC1)C2 ; *C[C@@H]1C[C@H](N2CCC(C(*)=O)CC2)CN1C ; *C[C@@H]1C[C@H](OCCCC(*)=O)CN1C ; *C[C@@H]1CC[C@H](CN2CCN(CC3CCN(*)CC3)CC2)N1C ; *C[C@@H]1CC[C@H](CN2CCN(*)CC2)N1C ; *C[C@@H]1C[C@H](N2CCC(C(=O)NCCCC(*)=O)CC2)CN1C]

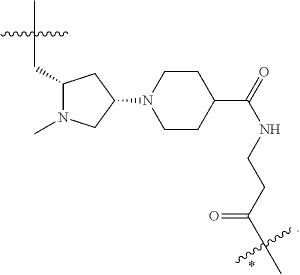 [SMILES: *C[C@@H]1C[C@H](N2CCC(C(=O)NCCC(*)=O)CC2)CN1C]

19. A pharmaceutical composition comprising the compound of any one of claims 1 to 18, or a pharmaceutically acceptable salt thereof; and a pharmaceutically acceptable carrier.

20. A method of treating cancer comprising administering to a subject in need a therapeutically effective amount of a compound of any one of claims 1 to 18, or a pharmaceutically acceptable salt thereof, or the pharmaceutical composition of claim 19.
